(19) United States
(12) Patent Application Publication
(10) Pub. No.: US 2019/0037820 A1
(43) Pub. Date: Feb. 7, 2019

(54) TRANSGENIC SILKWORM HAVING MAMMALIAN-TYPE SUGAR CHAIN ATTACHED THERETO

(71) Applicant: OSAKA UNIVERSITY, Osaka (JP)

(72) Inventors: Kazuhito FUJIYAMA, Suita-shi, Osaka (JP); Ryo MISAKI, Suita-shi, Osaka (JP); Ken-ichiro TATEMATSU, Ibaraki (JP); Hideki SEZUTSU, Ibaraki (JP)

(21) Appl. No.: 16/074,667

(22) PCT Filed: Feb. 3, 2017
(86) PCT No.: PCT/JP2017/004105

§ 371 Date: Aug. 30, 2018

(30) Foreign Application Priority Data
Feb. 5, 2016 (JP) 2016-021352

(51) Int. Cl. A01K 67/033; C12N 9/10; C12N 15/866

(52) U.S. Cl. CPC A01K 67/0339; C12N 9/1051; C12N 15/866; A01K 2227/703; C12Y 204/01038; C12Y 204/99003; C12N 9/1081

(57) ABSTRACT

It is intended to develop and provide a technique of conveniently allowing a transgenic silkworm by itself and at an individual level to produce a recombinant protein having a mammalian-type sugar chain sialic acid attached thereto, without the need of a baculovirus expression system or oral and transdermal administration of sialic acid. An expression vector was developed which can induce the expression of a mammalian-type glycosylation-related gene group only in a silk gland such that the recombinant protein modified with the mammalian-type sugar chain has no adverse effect on the silkworm itself. A transgenic silkworm harboring the expression vector was prepared.

### TECHNICAL FIELD

The present invention relates to a transgenic silk-spinning insect, particularly, a transgenic silkworm, which can attach a mammalian-type sugar chain to a recombinant protein of interest, and an expression vector for producing the transgenic form.

### BACKGROUND ART

The production of recombinant proteins by use of gene recombination technology is very important for the development of new materials or materials of high value and the production industry of pharmaceuticals, cosmetics, etc. For example, pharmaceutical proteins occupied 45.9% of top 10 items of global pharmaceutical sales in 2010 (Non Patent Literature 1) and will presumably further continue to grow.

The production of recombinant proteins has conventionally employed microbes such as E. coli and yeast, and cultured cells such as insect cells and animal cells as hosts. The hosts are selected according to the structures, purposes, etc. of the proteins to be produced. In general, protein production systems using microbes permit highly efficient production at low cost, but are not suitable for large-scale production or the production of recombinant proteins having a complicated structure. On the other hand, protein production systems using cultured cells such as cultured mammalian cells are capable of producing recombinant proteins having a complicated structure. Hence, such protein production systems using cultured cells have heretofore been used typically in the production of biopharmaceuticals. However, the protein production systems using cultured cells cannot avoid huge capital investment and high-cost production using expensive culture media, etc. Continued use of biopharmaceuticals puts a large economic burden on patients. Therefore, it has been desired to develop a protein production system that can produce recombinant proteins having a complicated structure at low cost.

In recent years, silkworms (Bombyx mori) have received attention as a novel host for protein production systems that can solve the problems described above. The silkworms belonging to Lepidoptera insects have been industrially useful insects responsible for silk production since a long time ago. Their expectation as useful protein production systems has grown in recent years because of breakthrough in the gene recombination technology. Silk glands which produce and secrete silk at the larva stage of the silkworms are known to be able to synthesize a large amount of proteins in a short period. Accordingly, the exploitation of this ability of the silk glands to synthesize proteins enables a protein of interest to be produced in large amounts in the silk glands. In the case of using a silkworm as a protein production system, the gene recombination technology is essential which involves introducing a foreign gene encoding the protein of interest into silkworm cells to prepare transformants, i.e., transgenic silkworms. Fortunately, a technique of stably maintaining a foreign gene within the genome using transposon piggyBac has been established for the silkworms (Non Patent Literature 2). Protein production systems using the silkworms are superior to protein production systems using other hosts in that, for example: the amount of proteins produced can be easily controlled by the number of silkworms reared; even several tens of thousands of silkworms can be reared in reduced space; the period from hatching to the late stage of the fifth instar larvae or pupae is as relatively short as a little less than 1 month; the silkworms can be reared throughout the year using an artificial hatching technique and an artificial diet; and produced proteins are easily recovered as cocoons.

Meanwhile, proteins produced in silkworms differ in the structures of sugar chains to be attached thereto from those produced in mammals including humans. As for N-linked sugar chains attached to proteins via their asparagine residues as shown in FIG. 1, proteins produced in silkworm pupae, midgut, fat body, etc. typically have a high mannose-type terminal structure where mannose is attached to a non-reducing terminal (FIG. 1B). Silk gland proteins produced in the silk glands of silkworms have a GlcNAc-terminated structure where N-acetylglucosamine (in the present specification, also referred to as “GlcNAc”) is further attached to the terminal mannose at the non-reducing terminal (FIG. 1C). By contrast, non-reducing terminals of sugar chains typically found in mammals assume a sialic acid-terminated structure where galactose and sialic acid are further attached to the GlcNAc (FIG. 1A).

Sugar chains are attached as one of the posttranslational modifications to proteins and are present in 50% or more of in vivo proteins. Such sugar chains play an important role in imparting various functions, such as protein stabilization, protection, physiological activity, antigen-antibody reaction, involvement in viral infection and pharmacokinetics, etc., to proteins. However, the difference in sugar chain structure between silkworms and mammals might exhibit immunogenicity and may become responsible for the onset of allergic response. Thus, in the case of producing pharmaceutical proteins with silkworms as a host, the difference in sugar chain structure is associated with the risk of influencing the activity or stability of the pharmaceutical proteins. Hence, a technique of engineering sugar chains of recombinant proteins produced in transgenic silkworms into mammalian-type sugar chains has been desired.

In the research using cultured insect cells, it has been reported that cultured cells which allow mammalian-type sugar chain modification with attached sialic acid at non-reducing terminals of recombinant proteins by introducing a human-type sugar chain modification pathway into the cultured cells (Non Patent Literature 3).

There is also a report stating that sialic acid was attached to sugar chain non-reducing terminals of recombinant proteins produced by baculovirus when an inhibitor of hexosaminidase, which decomposes N-acetylhexosamine in glycoprotein sugar chains, was added to a medium (Non Patent Literature 4).

Patent Literature 1 discloses that galactose was able to be attached to non-reducing terminals of N-linked sugar chains at an individual level of a silkworm by introducing β1,4-galactosyltransferase gene to silkworms. However, any transgenic silkworm provided with a function of attaching sialic acid to the non-reducing terminal of the galactose has not been known.

Non Patent Literature 5 discloses that sugar chains were successfully engineered by expressing human-derived β1,3-N-acetylglucosaminyltransferase 2 in silkworms. However, mammalian-type sialic acid was not attached to the non-reducing terminal.

### CITATION LIST

### Patent Literature

Patent Literature 1: JP Patent Publication (Kokai) No. 2014-012024 A (2014)

### Non Patent Literature

Non Patent Literature 1: 2012 Documents of Kansai Branch, Development Bank of Japan Inc.
Non Patent Literature 2: Tamura T. et al., 2000, Nat Biotechnol, 18: 81-84
Non Patent Literature 3: Jarivis D L et al., Curr Opin Biotechnol. 1998 October; 9 (5): 528-533
Non Patent Literature 4: Watanabe et al., J Biol Chem. 2002 Feb. 15; 277 (7): 5090-5093
Non Patent Literature 5: Dojima et al., J. Biotechnol. 2009, 143 (1): 27-33

### SUMMARY OF INVENTION

### Technical Problem

Conventional inventions have successfully attached sialic acid to non-reducing terminals of recombinant proteins by the combination of a transgenic silkworm, a baculovirus expression system and oral or transdermal administration of sialic acid. However, there has been a demand for a technique of introducing a sialic acid-attaching function by a more convenient method, i.e., by a transgenic silkworm by itself. Unfortunately, sialic acid-attached human-type sugar chains have an adverse effect on silkworm individuals and inhibit the development of silkworms by usual gene expression systems.

### Solution to Problem

In order to solve the problems described above, the present inventors have developed an expression vector that can induce the expression of a glycosylation-related gene group derived from a mammal such as a human only in a silk gland of a silkworm. A transgenic silkworm harboring the expression vector was able to efficiently attach galactose and sialic acid to a N-linked sugar chain terminal of a recombinant protein of interest produced in a silk gland, without being adversely affected in terms of development. The present inventors have also found that: the introduction of galactosyltransferase gene and three or more genes selected from a group consisting of enzyme genes related to a sialic acid synthesis system suffices for the attachment of a mammalian-type sugar chain; glycosylation efficiency is significantly increased by introducing CMP-Neu5Ac transporter gene to a silkworm; and regarding an isozyme of β1,4-galactosyltransferase, GalT2 has higher transfer efficiency than that of GalT1 generally used so far. The present invention is based on these results and findings and specifically provides the following aspects (1) to (20).

(1) A mammalian-type glycosylation agent comprising one to three independent expression vector(s) comprising a silk-spinning insect-derived middle and/or posterior silk gland promoter (MSG or PSG promoter) and (a) a gene encoding β1,4-galactosyltransferase (GalT) or a nucleotide encoding an active fragment of the enzyme, functionally linked downstream of the promoter, and (b) genes encoding three or more proteins selected from the group consisting of UDP-acetylglucosamine 2-epimerase/N-acetylmannosamine kinase (GNE), Neu5Ac9-phosphate synthase (NANS), Neu5Ac9-phosphate phosphatase (NANP), CMP-Neu5Ac synthase (CAMS), and a2,6-sialyltransferase (ST6GAL1) or nucleotides encoding active fragments of the proteins, wherein the genes encoding the proteins or the nucleotides encoding active fragments of the proteins are arranged so as to be under direct or indirect expression control of the MSG or PSG promoter.

(2) The mammalian-type glycosylation agent according to (1), wherein the β1,4-galactosyltransferase is GalT2.

(3) The mammalian-type glycosylation agent according to (1) or (2), wherein the middle silk gland promoter is a promoter of sericin 1 gene, sericin 2 gene, or sericin 3 gene.

(4) The mammalian-type glycosylation agent according to (1) or (2), wherein the posterior silk gland promoter is a promoter of fibroin H chain gene, fibroin L chain gene, or p25 gene.

(5) The mammalian-type glycosylation agent according to any of (1) to (4), wherein the three or more proteins selected are three or more proteins comprising GNE, CAMS, and ST6GAL1.

(6) The mammalian-type glycosylation agent according to any of (1) to (5), wherein the expression vector(s) further comprises (c) a gene encoding CMP-Neu5Ac transporter (SLC35A1) or a nucleotide encoding an active fragment of the enzyme.

(7) The mammalian-type glycosylation agent according to any of (1) to (6), wherein the expression vectors consist of a first expression vector comprising the gene or nucleotide described in the (a), and a second expression vector comprising the genes or nucleotides described in the (b).

(8) The mammalian-type glycosylation agent according to (7), wherein the gene or nucleotide (c) is comprised in the second expression vector.

(9) The mammalian-type glycosylation agent according to any of (1) to (6), wherein the genes encoding the proteins or the nucleotides encoding active fragments of the proteins are functionally linked downstream of the MSG or PSG promoter.

(10) The mammalian-type glycosylation agent according to any of (1) to (8), wherein the expression vector(s) is constituted by (i) a first subunit comprising the MSG or PSG promoter, and a gene encoding a transcriptional control element, functionally linked downstream of the promoter, and (ii) one or more second subunit(s) comprising a target promoter of the transcriptional control element, and the genes or nucleotides (a) to (c) functionally linked downstream of the promoter.

(11) The mammalian-type glycosylation agent according to (10), wherein the transcriptional control element is yeast-derived GAL4 protein, and the target promoter thereof is UAS (upstream activating sequence).

(12) The mammalian-type glycosylation agent according to any of (1) to (11), wherein the silk-spinning worm is a silkworm.

(13) The mammalian-type glycosylation agent according to any of (1) to (12), wherein the mammalian type is a human type.

(14) A transgenic silk-spinning insect capable of glycosylation with a mammalian-type sugar chain, comprising an expression vector(s) constituting a mammalian-type glycosylation agent according to any of (1) to (9).

(15) A transgenic silk-spinning insect capable of glycosylation with a mammalian-type sugar chain, comprising an expression vector(s) constituting a mammalian-type glycosylation agent according to (10) or (11).

(16) The transgenic silk-spinning insect capable of glycosylation with a mammalian-type sugar chain according to (15), wherein the first subunit and the second subunit reside on different chromosomes.

(17) The transgenic silk-spinning insect capable of glycosylation with a mammalian-type sugar chain according to any of (14) to (16), wherein the silk-spinning insect is a silkworm.

(18) The transgenic silk-spinning insect capable of glycosylation with a mammalian-type sugar chain according to any of (14) to (17), wherein the mammalian type is a human type.

(19) A line producing a transgenic silk-spinning insect capable of glycosylation with a mammalian-type sugar chain, comprising only a second subunit(s) of an expression vector(s) constituting a mammalian-type glycosylation agent according to (10) or (11).

(20) A method for preparing a transgenic silk-spinning insect capable of glycosylation with a mammalian-type sugar chain, comprising: a mating step of mating a line producing a transgenic silk-spinning insect capable of glycosylation with a mammalian-type sugar chain according to (19), and a line producing transgenic silk-spinning insect of the same species thereas having a first subunit(s) of an expression vector(s) constituting a mammalian-type glycosylation agent according to (10) or (11); and a selection step of selecting a transgenic silk-spinning insect comprising the first subunit and the second subunit as the transgenic silk-spinning insect capable of glycosylation with a mammalian-type sugar chain from a first filial generation (F1).

The present specification encompasses the contents disclosed in Japanese Patent Application No. 2016-21352 on which the priority of the present application is based.

### Advantageous Effects of Invention

According to the mammalian-type glycosylation agent of the present invention, a silk-spinning insect can be easily engineered into a transgenic silk-spinning insect capable of glycosylation with a mammalian-type sugar chain, by introducing the mammalian-type glycosylation agent to the silk-spinning insect, preferably a silkworm.

The transgenic silk-spinning insect capable of glycosylation with a mammalian-type sugar chain according to the present invention can attach a mammalian type N-linked sugar chain to a recombinant protein or peptide of interest produced in a silk gland of the transgenic silk-spinning insect.

The method for producing a transgenic silk-spinning insect capable of glycosylation with a mammalian-type sugar chain according to the present invention can produce the transgenic silk-spinning insect of interest capable of glycosylation with a mammalian-type sugar chain by administering the mammalian-type glycosylation agent of the present invention to a host silk-spinning insect and thereby introducing the expression vector(s) constituting the mammalian-type glycosylation agent to the silk-spinning insect.

### BRIEF DESCRIPTION OF DRAWINGS

FIG. 1 is a conceptual diagram of N-linked sugar chains of a mammal and a silkworm silk gland protein. FIG. 1A shows the mammalian N-linked sugar chain structure.

FIG. 1B shows the N-linked sugar chain structure of a protein present in a silkworm pupa, midgut, fat body, or the like. FIG. 1C shows the N-linked sugar chain structure of a protein present in a silk gland.

FIG. 2 is a conceptual diagram of mammalian type and insect-type N-linked sugar chain synthesis pathways in the Golgi apparatus and a human sialic acid synthesis pathway. In the diagram, the italicized genes encoding seven proteins (β1,4-galactosyltransferase GalT and six sialic acid-related proteins GNE, NANS, NANP, CMAS, ST6GAL1 and SLC35A1) are a gene group forcedly expressed in a silkworm individual in the present invention. The pathway indicated by broken line is a conceptual diagram of a reaction pathway that occurs in the present invention.

FIG. 3 shows results of SDS-PAGE of purified ATIII. Lane 1 shows a middle silk gland (MSG) extract, and lanes 2 to 7 each show an eluate of Ni column purification. The arrow indicates the position of ATIII. A region excised for intra-gel digestion is boxed.

FIG. 4 shows results of SDS-PAGE of purified IFNγ. Lane 1 shows a MSG extract, lane 2 shows a flow-through fraction of Ni column purification, lanes 3 and 4 each show a washing solution, and lanes 5 and 6 each show an eluate. The arrows indicate three detected bands. * and ** indicate bands excised for intra-gel digestion.

### DESCRIPTION OF EMBODIMENTS

### 1. Mammalian-Type Glycosylation Agent

### 1-1. Summary

The first aspect of the present invention is a mammalian-type glycosylation agent. The glycosylation agent of the present invention is constituted by one to three independent expression vector(s). A transgenic silk-spinning insect capable of glycosylation with a mammalian-type sugar chain can be easily prepared by introducing the glycosylation agent of the present invention into a silk-spinning insect such as a silkworm.

### 1-2. Definition

The following terms frequently used in the present specification will be defined.

In the present specification, the “mammalian-type glycosylation agent” refers to an agent that has a configuration mentioned later and is applied to a silk-spinning insect.

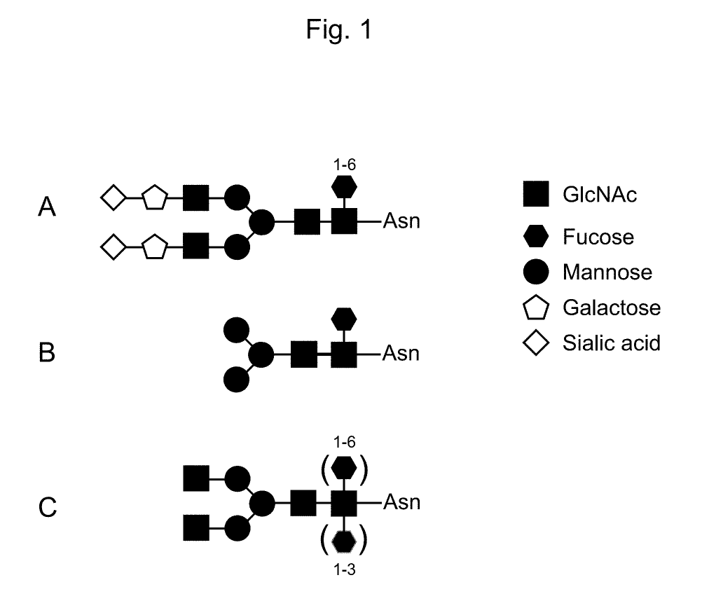

In the present specification, the “mammalian-type sugar chain” is a N-linked sugar chain attached to a protein via its asparagine residue and is a sugar chain having a sugar chain structure with sialic acid at a non-reducing terminal where, as shown in FIG. 1A, galactose is attached to a GlcNAc non-reducing terminal and sialic acid is further attached to the galactose.

In general, the mammalian-type sugar chain has a structure represented by SiaₙGalₙGlcNAcₙManₘ-Asn (Sia represents sialic acid, particularly, N-acetylneuraminic acid, Gal represents galactose, Man represents mannose, each m independently represents an integer of 2 or lager, and each n independently represents an integer of 1 or larger).

In the present invention, the “mammal” is not particularly limited. Preferably, a human, a chimpanzee, a rat, a mouse, a dog, a cat, cattle, a pig, a horse, a goat, sheep or the like corresponds thereto. A human is preferred. That is, a “human-type sugar chain” is preferred.

The “silk” typically refers to animal fiber that is biosynthesized in a silk gland of a silk-spinning insect.

In the present specification, the “silk-spinning insect” refers to a generic name for insects that have silk glands and can spin silk. Specifically, the silk-spinning insect typically refers to a Lepidoptera insect, a Hymenoptera insect, a Neuroptera insect, a Trichoptera insect or the like of type that can spin for nesting, cocooning or moving at the larva stage. In this context, the Lepidoptera insect is an insect taxonomically belonging to the order Lepidoptera, and various butterflies or moths correspond thereto. The Hymenoptera insect is an insect taxonomically belonging to the order Hymenoptera, and various bees or ants correspond thereto. The Neuroptera insect is an insect belonging to the order Neuroptera, and dobsonflies, owlflies, ant lions or the like correspond thereto. The Trichoptera insect is an insect belonging to the order Trichoptera, and various caddice-flies correspond thereto. The silk-spinning insect according to the present invention is preferably a Lepidoptera insect, which has large silk glands and can spin a large amount of silk. Among others, a species belonging to the family Bombycidae, Saturniidae, Brahmaeidae, Eupterotidae, Lasiocampidae, Psychidae, Arctiidae, Noctuidae or the like is preferred. A species belonging to the genus Bombyx, Samia, Antheraea, Saturnia, Attacus, or Rhodinia, specifically, a species belonging to a group called wild silkworm such as a silkworm as well as Bombyx mandarina, Samia cynthia (including Samia cynthia ricini and hybrids of Samia cynthia and Samia cynthia ricini), Antheraea yamamai, Antheraea pernyi, Saturnia japonica, and Actias gnoma, is particularly preferred. The silk-spinning insect is most preferably a silkworm.

The “silk gland” is a fistula that is a modified salivary gland of a silk-spinning insect, and has a function of producing, accumulating, and secreting liquid silk. The silk gland is typically present as a pair of right and left silk glands along the larval digestive tract of the silk-spinning insect. Each silk gland is constituted by 3 regions, anterior, middle and posterior silk glands. In many silk-spinning insects including silkworms, a water-soluble gelatin-like protein sericin serving as a coating component of silk is synthesized in middle silk gland (in the present specification, also referred to as “MSG”) cells and secreted into the middle silk gland lumen. Also, three major proteins, fibroin H chain (in the present specification, also referred to as “Fib H”), fibroin L chain (in the present specification, also referred to as “Fib L”), and p25/FHX (hereinafter, referred to as “p25”), which constitute fiber components of silk are synthesized in posterior silk gland (in the present specification, also referred to as “PSG”) cells. These three proteins form a SFEU (silk fibroin elementary unit) complex at a ratio of Fib H:Fib L:p25=6:6:1, which is then secreted into the posterior silk gland lumen. Then, the SFEU complex is migrated to the middle silk gland lumen, coated with sericin, and spun as silk from the anterior silk gland. Thus, in the case of using a silk-spinning insect as a protein expression system, a gene expression system specifically expressed in the middle or posterior silk gland can be used.

In the present specification, the “expression vector” refers to an expression unit that comprises a recombinant gene encoding a recombinant protein or a nucleotide encoding an active fragment thereof in an expressible state and can control the expression of the recombinant gene, etc. The expression vector of the present invention can employ various expression units capable of replicating in host cells. Examples thereof include plasmid vectors and Bacmid vectors capable of autonomously replicating, and viral vectors. In the present specification, a plasmid vector is typically used. The expression vector constituting the mammalian-type glycosylation agent of the present invention (hereinafter, in the present specification, also referred to as a “glycosylation agent expression vector”) may be constituted by two or more independent subunits. In this case, all of the subunits are interpreted as one expression vector. The configuration of each subunit will be mentioned later.

In the present specification, the “recombinant protein of interest” is a protein encoded by the recombinant gene of interest and refers to a recombinant protein to be produced in a silk gland in a protein production system using a silk-spinning insect. In the present specification, the recombinant protein of interest is a recombinant protein glycosylated with a mammalian-type sugar chain in a transgenic silk-spinning insect capable of glycosylation with a mammalian-type sugar chain. The recombinant protein of interest may be derived from one gene or a gene fragment thereof, or may be derived from a chimeric gene containing linked portions of a plurality of genes. The amino acid length of the recombinant protein of interest is not particularly limited. The number of amino acid residues may be 8 to 10,000. The recombinant protein of interest also encompasses, for example, a peptide hormone consisting of only 9 amino acids, such as oxytocin. In the present specification, the type of the recombinant protein of interest is not particularly limited, and a protein of high value is preferred. Examples thereof include: peptide hormones such as insulin, calcitonin, parathormone and growth hormone; cytokines such as epidermal growth factor (EGF), fibroblast growth factor (FGF), interleukin (IL), interferon (IFN), tumor necrosis factor α (TNF-α) and transforming growth factor β (TGF-β); and immunoglobulin, antithrombin III, serum albumin, hemoglobin, various enzymes, and collagen, and their fragments (including chimeric peptides).

In the present specification, the “recombinant gene of interest” refers to a foreign gene, in principle, encoding the recombinant protein of interest described above. In the present specification, the recombinant gene of interest is present in an expression vector such that the recombinant gene of interest is functionally linked to a middle and/or posterior silk gland promoter. This expression vector may be a glycosylation agent expression vector.

### 1-3. Configuration

1-3-1. Component

The mammalian-type glycosylation agent of the present invention is constituted by a glycosylation agent expression vector. The glycosylation agent expression vector comprises (1) a silk-spinning insect-derived middle and/or posterior silk gland promoter and (2) glycosylation-related genes or nucleotides encoding active fragments of proteins encoded thereby (in the present specification, also referred to as “glycosylation-related genes, etc.”) as essential components. When the glycosylation agent expression vector is constituted by two subunits, a first subunit and a second subunit mentioned later, the glycosylation agent expression vector can contain (3) a gene encoding a transcriptional control element and (4) a target promoter of the transcriptional control element as essential components. In addition, the glycosylation agent expression vector can comprise other components capable of contributing to the expression of the glycosylation-related genes, etc. Examples of such other components include (5) a terminator, (6) a marker gene, (7) an enhancer, (8) an insulator, and (9) an inverted terminal repeat sequence of transposon. Hereinafter, each component will be specifically described.

(1) Middle or Posterior Silk Gland Promoter

In the present specification, the “middle or posterior silk gland promoter (MSG or PSG promoter)” is an essential component of the glycosylation agent expression vector and refers to a site-specific promoter that controls the expression of a gene specifically expressed in a middle or posterior silk gland of a silk-spinning insect.

Examples of the gene specifically expressed in the middle silk gland (MSG) of a silk-spinning insect include sericin 1 (in the present specification, also referred to as “Ser1”) gene, sericin 2 (in the present specification, also referred to as “Ser2”) gene, and sericin 3 (in the present specification, also referred to as “Ser3”) gene. Thus, promoters that control the expression of Ser1 to Ser3 genes (in the present specification, referred to as Ser1 promoter, Ser2 promoter, and Ser3 promoter, respectively) are preferred as the silk-spinning insect-derived MSG promoter of the glycosylation agent expression vector. Specific examples of these promoters include silkworm-derived Ser1 promoter consisting of the nucleotide sequence represented by SEQ ID NO: 1, Ser2 promoter consisting of the nucleotide sequence represented by SEQ ID NO: 2, and Ser3 promoter consisting of the nucleotide sequence represented by SEQ ID NO: 3.

Examples of the gene specifically expressed in the posterior silk gland (PSG) of a silk-spinning insect include Fib H gene, Fib L gene, and p25 gene. Thus, promoters that control the expression of these genes (in the present specification, referred to as Fib H promoter, Fib L promoter, and p25 promoter, respectively) are preferred as the silk-spinning insect-derived PSG promoter of the glycosylation agent expression vector. Specific examples of these promoters include silkworm-derived Fib H promoter consisting of the nucleotide sequence represented by SEQ ID NO: 4, Fib L promoter consisting of the nucleotide sequence represented by SEQ ID NO: 5, and p25 promoter consisting of the nucleotide sequence represented by SEQ ID NO: 6, and tussah-derived Fib H promoter consisting of the nucleotide sequence represented by SEQ ID NO: 7 and Fib L promoter consisting of the nucleotide sequence represented by SEQ ID NO: 8.

The nucleotide sequence of the MSG or PSG promoter is evolutionarily conserved very well among silk-spinning insects. Thus, for example, the PSG promoter is highly probably operable in PSGs of different silk-spinning insect species (Sezutsu H., et al., 2009, Journal of Insect Biotechnology and Sericology, 78: 1-10). Accordingly, the organism species from which the MSG or PSG promoter is derived is not necessarily required to be the same as the organism species of a silk-spinning insect to which the glycosylation agent expression vector is to be introduced. The promoter is preferably derived from a species belonging to the same order thereas, more preferably a species belonging to the same family thereas, further preferably a species belonging to the same genus thereas, most preferably the same species thereas.

The glycosylation agent expression vector may comprise either of the MSG or PSG promoter. As mentioned later, when the glycosylation agent expression vector is constituted by two independent expression vectors, these expression vectors may comprise different silk gland promoters. Alternatively, different silk gland promoters may control the expression of a plurality of glycosylation-related genes mentioned later comprised in one expression vector. Usually, any one of the MSG and PSG promoters suffices. The MSG promoter is preferred.

The MSG or PSG promoter in the glycosylation agent expression vector is configured such that the glycosylation-related genes, etc. or a gene encoding a transcriptional control element mentioned later can be arranged within the scope of a control region downstream thereof (on the 3′-terminal side).

(2) Glycosylation-Related Gene, Etc.

The glycosylation-related genes or nucleotides encoding active fragments of proteins encoded by the genes (glycosylation-related genes, etc.) are core components in the glycosylation agent expression vector together with the MSG or PSG promoter mentioned above.

In the present specification, the “glycosylation-related genes” refer to genes encoding β1,4-galactosyltransferase and six sialic acid-related proteins related to mammalian-type glycosylation. The “nucleotides encoding active fragments of proteins encoded by the genes” refer to nucleotides encoding the amino acid sequences of peptides that are partial peptides of the proteins encoded by the glycosylation-related genes and have physiological activity equivalent to or higher than the proteins. For example, nucleotides encoding functional domains of the proteins correspond thereto. The length of amino acids in the partial peptides is not particularly limited as long as the partial peptides have activity. The amino acid length can be 40 or more amino acids, 50 or more amino acids, 60 or more amino acids, or 70 or more amino acids and less than the full length. Hereinafter, each glycosylation-related gene will be specifically described.

A. β1,4-Galactosyltransferase

The “β1,4-galactosyltransferase (in the present specification, also referred to as “GalT”)” is an enzyme that catalyzes the reaction of transferring galactose from a donor substrate UDP-galactose (UDP-Gal) to GlcNAcβ1-2Man of a glycoprotein. In the mammalian-type glycosylation agent of the present invention, this enzyme has a function of attaching galactose to the GlcNAc non-reducing terminal of a N-linked sugar chain in a silk gland-derived protein of a silk-spinning insect. GalT is known to have a plurality of isozymes. For example, 7 types of isozymes have been identified in mice. Among them, 4 types, GalT1, GalT2, GalT3, and GalT4, are involved in glycoproteins, and 3 types, GalT1, GalT2, and GalT3, have activity as the mammalian-type glycosylation agent of the present invention. Among them, GalT2 is particularly preferred. Thus, the term “GalT” described in the present specification means any of GalT1, GalT2, and GalT3 unless otherwise specified.

The GalT gene is an essential component in the glycosylation agent expression vector. The organism species from which the GalT gene in the glycosylation agent expression vector is derived is not particularly limited. For example, the GalT gene derived from any organism such as an invertebrate (nematode, insect, etc.), a chordate, or a vertebrate (fish, amphibian, reptile, bird, mammal, etc.) can be used. The GalT gene derived from a mammal such as a human, a rat, or a mouse is preferred. Specific examples thereof include human GalT1 gene encoding human GalT1 consisting of the amino acid sequence represented by SEQ ID NO: 9 (e.g., human GalT1 gene consisting of the nucleotide sequence represented by SEQ ID NO: 10), rat GalT1 gene encoding rat GalT1 consisting of the amino acid sequence represented by SEQ ID NO: 11 (e.g., rat GalT1 gene consisting of the nucleotide sequence represented by SEQ ID NO: 12), mouse GalT1 gene encoding mouse GalT1 consisting of the amino acid sequence represented by SEQ ID NO: 13 (e.g., mouse GalT1 gene consisting of the nucleotide sequence represented by SEQ ID NO: 14), human GalT2 gene encoding human GalT2 consisting of the amino acid sequence represented by SEQ ID NO: 15 (e.g., human GalT2 gene consisting of the nucleotide sequence represented by SEQ ID NO: 16), rat GalT2 gene encoding rat GalT2 consisting of the amino acid sequence represented by SEQ ID NO: 17 (e.g., rat GalT1 gene consisting of the nucleotide sequence represented by SEQ ID NO: 18), mouse GalT2 gene encoding mouse GalT2 consisting of the amino acid sequence represented by SEQ ID NO: 19 (e.g., mouse GalT2 gene consisting of the nucleotide sequence represented by SEQ ID NO: 20), human GalT3 gene encoding human GalT3 consisting of the amino acid sequence represented by SEQ ID NO: 21 (e.g., human GalT3 gene consisting of the nucleotide sequence represented by SEQ ID NO: 22), rat GalT3 gene encoding rat GalT3 consisting of the amino acid sequence represented by SEQ ID NO: 23 (e.g., rat GalT3 gene consisting of the nucleotide sequence represented by SEQ ID NO: 24), and mouse GalT3 gene encoding mouse GalT3 consisting of the amino acid sequence represented by SEQ ID NO: 25 (e.g., mouse GalT3 gene consisting of the nucleotide sequence represented by SEQ ID NO: 26).

B. Six Sialic Acid-Related Proteins

The “six sialic acid-related proteins” are a series of proteins necessary for attaching sialic acid to the non-reducing terminal consisting of the galactose at a N-linked sugar chain in silk gland cells of a silk-spinning insect and consist of four α2,6-sialyltransferase substrate synthesis-related enzymes, α2,6-sialyltransferase, and sugar nucleotide transporter.

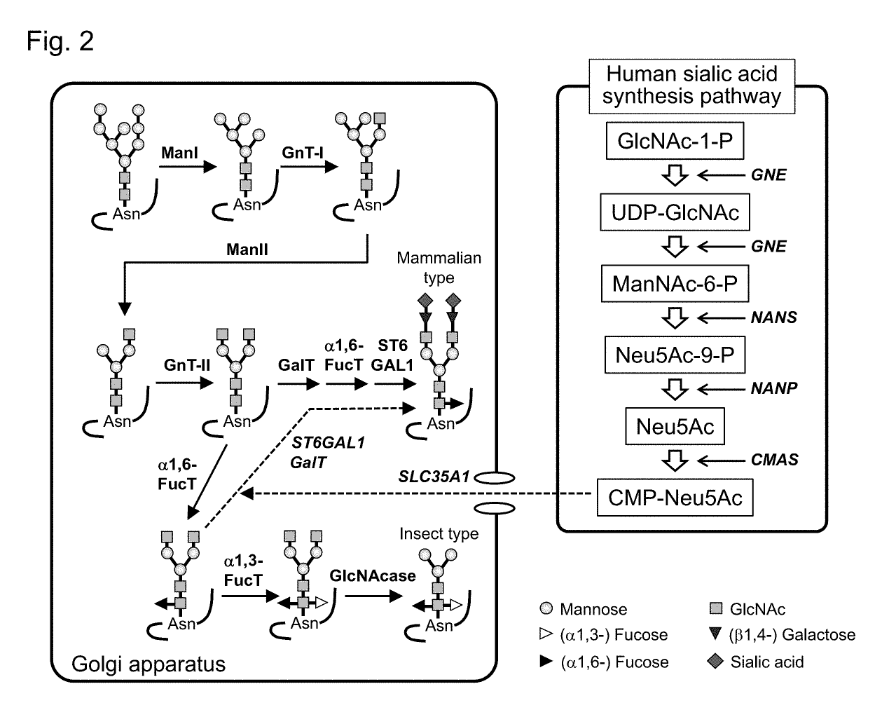

The four α2,6-sialyltransferase substrate synthesis-related enzymes are an enzyme group that functions in the sialic acid synthesis pathway of converting GlcNAc-1-P (N-acetylglucosamine-1-phosphate) to CMP-Neu5Ac (CMP-N-acetylneuraminic acid:CMP-sialic acid), as shown in FIG. 2, and consist of UDP-acetylglucosamine 2-epimerase/N-acetylmannosamine kinase, Neu5Ac9-phosphate synthase, Neu5Ac9-phosphate phosphatase, and CMP-Neu5Ac synthase.

In the present specification, the genes encoding the sialic acid-related proteins are also referred to as “sialic acid-related genes”. Hereinafter, the six sialic acid-related proteins will be described.

The “UDP-acetylglucosamine 2-epimerase/N-acetylmannosamine kinase (in the present specification, also referred to as “GNE”)” has a catalytic effect of converting GlcNAc-1-P to UDP-GlcNAc (uridine diphosphate N-acetylglucosamine) and further the UDP-GlcNAc to ManNac-6-P (N-acetylmannosamine-6-phosphate). In the present specification, the gene encoding GNE is referred to as “GNE gene”. The organism species from which the GNE gene in the glycosylation agent expression vector is derived is not particularly limited. The GEN gene derived from any organism such as an invertebrate (nematode, insect, etc.), a chordate, or a vertebrate (fish, amphibian, reptile, bird, mammal, etc.) can be used. The GNE gene derived from a mammal such as a human, a rat, or a mouse is preferred. Specific examples thereof include a gene encoding human GNE consisting of the amino acid sequence represented by SEQ ID NO: 27 (e.g., human GNE gene consisting of the nucleotide sequence represented by SEQ ID NO: 28), rat GNE gene encoding rat GNE consisting of the amino acid sequence represented by SEQ ID NO: 29 (e.g., rat GNE gene consisting of the nucleotide sequence represented by SEQ ID NO: 30), and mouse GNE gene encoding mouse GNE consisting of the amino acid sequence represented by SEQ ID NO: 31 (e.g., mouse GNE gene consisting of the nucleotide sequence represented by SEQ ID NO: 32).

The “Neu5Ac9-phosphate synthase (in the present specification, also referred to as “NANS”)” has a catalytic effect of converting ManNac-6-P formed by the catalytic effect of GNE to Neu5Ac-9-P (N-acetylneuraminic acid-9-phosphate). In the present specification, the gene encoding NANS is referred to as “NANS gene”. The organism species from which the NANS gene in the glycosylation agent expression vector is derived is not particularly limited. The NANS gene derived from any organism such as an invertebrate (nematode, insect, etc.), a chordate, or a vertebrate (fish, amphibian, reptile, bird, mammal, etc.) can be used. The NANS gene derived from a mammal such as a human, a rat, or a mouse is preferred. Specific examples thereof include human NANS gene encoding human NANS consisting of the amino acid sequence represented by SEQ ID NO: 33 (e.g., human NANS gene consisting of the nucleotide sequence represented by SEQ ID NO: 34), rat NANS gene encoding rat NANS consisting of the amino acid sequence represented by SEQ ID NO: 35 (e.g., rat NANS gene consisting of the nucleotide sequence represented by SEQ ID NO: 36), and mouse NANS gene encoding mouse NANS consisting of the amino acid sequence represented by SEQ ID NO: 37 (e.g., mouse NANS gene consisting of the nucleotide sequence represented by SEQ ID NO: 38).

The “Neu5Ac9-phosphate phosphatase (in the present specification, also referred to as “NANP”)” has a catalytic effect of converting Neu5Ac-9-P formed by the catalytic effect of NANS to Neu5Ac (N-acetylneuraminic acid) by removing phosphoric acid. In the present specification, the gene encoding NANP is referred to as “NANP gene”. The organism species from which the NANP gene in the glycosylation agent expression vector is derived is not particularly limited. The NANP gene derived from any organism such as an invertebrate (nematode, insect, etc.), a chordate, or a vertebrate (fish, amphibian, reptile, bird, mammal, etc.) can be used. The NANP gene derived from a mammal such as a human, a rat, or a mouse is preferred. Specific examples thereof include human NANP gene encoding human NANP consisting of the amino acid sequence represented by SEQ ID NO: 39 (e.g., human NANP gene consisting of the nucleotide sequence represented by SEQ ID NO: 40), rat NANP gene encoding rat NANP consisting of the amino acid sequence represented by SEQ ID NO: 41 (e.g., rat NANP gene consisting of the nucleotide sequence represented by SEQ ID NO: 42), and mouse NANP gene encoding mouse NANP consisting of the amino acid sequence represented by SEQ ID NO: 43 (e.g., mouse NANP gene consisting of the nucleotide sequence represented by SEQ ID NO: 44).

The “CMP-Neu5Ac synthase (in the present specification, also referred to as “CMAS”)” has a catalytic effect of converting Neu5Ac formed by the catalytic effect of NANP to CMP-Neu5Ac. In the present specification, the gene encoding CMAS is referred to as “CMAS gene”. The organism species from which the CMAS gene in the glycosylation agent expression vector is derived is not particularly limited. The CMAS gene derived from any organism such as an invertebrate (nematode, insect, etc.), a chordate, or a vertebrate (fish, amphibian, reptile, bird, mammal, etc.) can be used. The CMAS gene derived from a mammal such as a human, a rat, or a mouse is preferred. Specific examples thereof include human CMAS gene encoding human CMAS consisting of the amino acid sequence represented by SEQ ID NO: 45 (e.g., human CMAS gene consisting of the nucleotide sequence represented by SEQ ID NO: 46), rat CMAS gene encoding rat CMAS consisting of the amino acid sequence represented by SEQ ID NO: 47 (e.g., rat CMAS gene consisting of the nucleotide sequence represented by SEQ ID NO: 48), and mouse CMAS gene encoding mouse CMAS consisting of the amino acid sequence represented by SEQ ID NO: 49 (e.g., mouse CMAS gene consisting of the nucleotide sequence represented by SEQ ID NO: 50).

The “α2,6-sialyltransferase (in the present specification, also referred to as “ST6GAL1”)” is a glycosyltransferase that catalyzes the reaction of transferring a sialic acid residue from the donor substrate CMP-Neu5Ac synthesized in the sialic acid synthesis pathway and transported into the Golgi apparatus by the action of SLC35A1 to a sugar chain structure in acceptor substrates glycoprotein and glycolipid. In the present specification, the gene encoding ST6GAL1 is referred to as “ST6GAL1 gene”. The organism species from which the ST6GAL1 gene in the glycosylation agent expression vector is derived is not particularly limited. The ST6GAL1 gene derived from any organism such as an invertebrate (nematode, insect, etc.), a chordate, or a vertebrate (fish, amphibian, reptile, bird, mammal, etc.) can be used. The ST6GAL1 gene derived from a mammal such as a human, a rat, or a mouse is preferred. Specific examples thereof include human ST6GAL1 gene encoding human ST6GAL1 consisting of the amino acid sequence represented by SEQ ID NO: 51 (e.g., human ST6GAL1 gene consisting of the nucleotide sequence represented by SEQ ID NO: 52), rat ST6GAL1 gene encoding rat ST6GAL1 consisting of the amino acid sequence represented by SEQ ID NO: 53 (e.g., rat ST6GAL1 gene consisting of the nucleotide sequence represented by SEQ ID NO: 54), and mouse ST6GAL1 gene encoding mouse ST6GAL1 consisting of the amino acid sequence represented by SEQ ID NO: 55 (e.g., mouse ST6GAL1 gene consisting of the nucleotide sequence represented by SEQ ID NO: 56).

In the present specification, the “sugar nucleotide transporter” refers to a transporter protein having the ability to transport sialic acid to the Golgi apparatus. For example, CMP-NeuSAc transporter corresponds thereto.

The “CMP-Neu5Ac transporter (in the present specification, also referred to as “SLC35A1”)” is a membrane transport protein present on the membrane of the Golgi apparatus and has an effect of transporting CMP-Neu5Ac synthesized in the sialic acid synthesis pathway into the Golgi apparatus. In the present specification, the gene encoding SLC35A1 is referred to as “SLC35A1 gene”. The organism species from which the SLC35A1 gene in the glycosylation agent expression vector is derived is not particularly limited. The SLC35A1 gene derived from any organism such as an invertebrate (nematode, insect, etc.), a chordate, or a vertebrate (fish, amphibian, mammal, etc.) can be used. The SLC35A1 gene derived from a mammal such as a human, a rat, or a mouse is preferred. Specific examples thereof include human SLC35A1 gene encoding human SLC35A1 consisting of the amino acid sequence represented by SEQ ID NO: 57 (e.g., human SLC35A1 gene consisting of the nucleotide sequence represented by SEQ ID NO: 58), rat SLC35A1 gene encoding rat SLC35A1 consisting of the amino acid sequence represented by SEQ ID NO: 59 (e.g., rat SLC35A1 gene consisting of the nucleotide sequence represented by SEQ ID NO: 60), and mouse SLC35A1 gene encoding mouse SLC35A1 consisting of the amino acid sequence represented by SEQ ID NO: 61 (e.g., mouse SLC35A1 gene consisting of the nucleotide sequence represented by SEQ ID NO: 62).

Among the six sialic acid-related genes described above, three or more genes selected from the group consisting of five genes, the GNE gene, the NANS gene, the NANP gene, the CMAS gene and the ST6GAL1 gene, excluding the SLC35A1 gene are comprised as essential components in the glycosylation agent expression vector. Examples of the three genes include a set of the GNE gene, the CMAS gene and the ST6GAL1 gene. Examples of the four genes include a set of the GNE gene, the NANP gene, the CMAS gene and the ST6GAL1 gene. Preferably, all of the five genes are selected. On the other hand, the SLC35A1 gene is an optional component in the glycosylation agent expression vector. It is particularly preferred to comprise all of the six genes because the advantageous effects of the invention are enhanced by the addition of the SLC35A1 gene.

Each gene described above is preferably a wild-type gene and may be a variant gene as long as the activity of the protein encoded by each gene is maintained. Examples thereof include variant genes based on gene polymorphism such as SNPs. Examples of such variant genes include a gene consisting of a nucleotide sequence derived from the nucleotide sequence of the wild-type gene by the deletion, substitution or addition of one to several bases, and a gene consisting of a nucleotide sequence having 70% or higher, 80% or higher, 85% or higher or 90% or higher, preferably 95% or higher, more preferably 96% or higher, 97% or higher, 98% or higher or 99% or higher base identity to the nucleotide sequence of the wild-type gene. In the present specification, the term “several” refers to, for example, 2 to 20, 2 to 15, 2 to 10, 2 to 7, 2 to 5, 2 to 4 or 2 or 3. The “base identity” refers to the ratio (%) of identical bases between two nucleotide sequences to the total number of base residues in the wild-type gene when the two nucleotide sequences are aligned, with a gap introduced, if necessary, to any of the nucleotide sequences to attain the highest base similarity therebetween.

(3) Gene Encoding Transcriptional Control Element

In the present specification, the “gene encoding a transcriptional control element” is an essential component in a first subunit mentioned later and refers to a gene of a transcriptional control element. In the present specification, the “transcriptional control element” refers to a protein factor that can bind to a target promoter mentioned later and thereby activate the target promoter. Examples thereof include GAL4 protein which is a yeast galactose metabolism-activating protein, and tTA which is a tetracycline-controlled transcriptional activator, and variants thereof.

(4) Target Promoter of the Transcriptional Control Element

In the present specification, the “target promoter of the transcriptional control element” is an essential component in a second subunit mentioned later and refers to a promoter that can activate gene expression under its control through the binding of the transcriptional control element encoded in the first subunit thereto. The transcriptional control element and the target promoter thereof are in a correspondence relationship. Usually, if the transcriptional control element is determined, the target promoter thereof is also determined inevitably. For example, when the transcriptional control element is GAL4 protein, UAS (upstream activating sequence) is used.

The target promoter of the transcriptional control element in the glycosylation agent expression vector is configured such that the glycosylation-related genes, etc. mentioned above can be arranged within the scope of a control region downstream thereof.

(5) Terminator

In the present specification, the “terminator” is an optional component constituted by a nucleotide sequence that can terminate the transcription of a gene, etc. during its expression in the glycosylation agent expression vector of the present aspect.

(6) Marker Gene

In the present specification, the “marker gene” is a gene encoding a marker protein also called selection marker. The marker protein refers to a polypeptide that allows the presence or absence of the expression of the marker gene to be determined on the basis of its activity. Hence, when the glycosylation agent expression vector comprises the marker gene, a transgenic silk-spinning insect harboring the glycosylation agent expression vector can be easily determined on the basis of the activity of the marker protein. In this context, the phrase “on the basis of the activity” means “on the basis of results of detecting the activity”. The detection of the activity may directly detect the activity itself of the marker protein or may be indirect detection via a metabolite, such as a dye, formed by the activity of the marker protein. The detection may be any of chemical detection (including detection through enzymatic reaction), physical detection (including detection by behavior analysis), and sensory detection by a person in charge of detection (including visual, tactile, olfactory, auditory, and gustatory detection).

The type of the marker protein is not particularly limited as long as its activity is detectable by a method known in the art. A marker protein that is low invasive to a transgenic silkworm in detection is preferred. Examples thereof include fluorescent proteins, dye-synthesizing proteins, luminescent proteins, externally secreted proteins, and proteins that control external morphology, etc. A fluorescent protein, a dye-synthesizing protein, a luminescent protein, and an externally secreted protein are particularly preferred because these proteins are visually detectable under particular conditions and therefore very low invasive to a transgenic silkworm and permit easy determination and selection.

The fluorescent protein refers to a protein that emits fluorescence having a particular wavelength when a transgenic silkworm is irradiated with excitation light having a particular wavelength. Any of natural and non-natural fluorescent proteins may be used. The excitation wavelength and the fluorescence wavelength are not particularly limited. Specific examples thereof include CFP, AmCyan, RFP, DsRed (including derivatives such as DsRed monomer and DsRed2), YFP, and GFP (including derivatives such as EGFP and EYFP).

The dye-synthesizing protein is a protein involved in the biosynthesis of a dye and is usually an enzyme. In this context, the “dye” is a low-molecular compound or a peptide that can impart the dye to a transformant and is not limited by its type. A dye that appears as an external color of an individual is preferred. Examples thereof include melanin dyes (including dopamine melanin), ommochrome dyes, and pteridine-type dyes.

The luminescent protein refers to a substrate protein that can emit light without the need of excitation light, or an enzyme that catalyzes the luminescence of the substrate protein. Examples thereof include aequorin, and luciferase as an enzyme.

In the present specification, the externally secreted protein is a protein that is secreted to the outside of cells or the outside of the body, and an exocrine enzyme or the like corresponds thereto. An enzyme that contributes to the decomposition or inactivation of a drug such as blasticidin and imparts drug resistance to a host as well as a digestive enzyme corresponds to the exocrine enzyme.

The marker gene is placed in an expressible state downstream of a promoter in the glycosylation agent expression vector. The promoter used may be the same as or different from the middle or posterior silk gland promoter.

(7) Enhancer

The “enhancer” is a gene expression activation region that can increase the transcription level of a target gene in cooperation with a promoter and is constituted by a particular DNA sequence. Unlike a promoter, the enhancer is placed not only upstream (on the 5′-terminal side) of the target gene but downstream (on the 3′-terminal side) of or within the target gene to regulate the transcription of the target gene.

(8) Insulator

In the present specification, the “insulator” is an optional component in the glycosylation agent expression vector and is a nucleotide sequence that can stably control the transcription of a gene flanked by the insulator sequences without being influenced by the neighboring chromatin of chromosomes. Examples thereof include a chicken cHS4 sequence and a fruit fly gypsy sequence.

(9) Inverted Terminal Repeat Sequence of Transposon

In the present specification, the “inverted terminal repeat sequence (ITRS) of transposon” is an optional component that may be comprised when the glycosylation agent expression vector is an expression vector capable of homologous recombination. The inverted terminal repeat sequence is usually used as one set of two sequences, and piggyBac, mariner, minos, or the like can be used as the transposon (Shimizu, K. et al., 2000, Insect Mol. Biol., 9, 277-281; and Wang W. et al., 2000, Insect Mol Biol 9 (2): 145-155).

1-3-2. Unit Configuration of Glycosylation Agent Expression Vector

In the glycosylation agent expression vector constituting the mammalian-type glycosylation agent of the present invention, the glycosylation-related genes, etc. are arranged so as to be under direct or indirect expression control of the MSG or PSG promoter. In this context, the “direct or indirect expression control” means the positional relationship between the MSG or PSG promoter and the glycosylation-related genes, etc. in the glycosylation agent expression vector. This depends on the unit configuration of the glycosylation agent expression vector. The glycosylation agent expression vector may be constituted by one unit and may be constituted by two subunits. Hereinafter, each case will be described.

(1) Case of being Constituted by One Unit

The glycosylation agent expression vector comprises, in one glycosylation agent expression vector, all the components necessary for expressing the glycosylation-related genes, etc. in silk-spinning insect cells. Specifically, the glycosylation agent expression vector comprises the essential components, i.e., the MSG or PSG promoter and the glycosylation-related genes, etc. functionally linked downstream of the promoter.

In the present specification, the term “functionally linked” means that each of the glycosylation-related genes, etc. is integrated in an expressible state in the glycosylation agent expression vector. Specifically, the term means that each of the glycosylation-related genes, etc. is arranged downstream of the MSG or PSG promoter under the control of the MSG or PSG promoter in the glycosylation agent expression vector. Thus, when the glycosylation agent expression vector is constituted by one unit, the glycosylation-related genes, etc. are to be under direct expression control of the MSG or PSG promoter.

When the glycosylation agent expression vector is constituted by one unit, a transgenic silk-spinning insect having the glycosylation agent expression vector can constantly attach a mammalian-type sugar chain to a recombinant protein produced in a silk gland.

(2) Case of being Constituted by Two Subunits

When the glycosylation agent expression vector is constituted by two subunits, a first subunit and a second subunit, the components essential for the expression of the glycosylation-related genes, etc. are divided in these subunits. Thus, this configuration functions as one glycosylation agent expression vector only when the first and second subunits coexist with each other in a silk-spinning insect cell of a host. Specifically, in the same cell, the transcriptional control element is expressed from the first subunit by the activation of the promoter comprised in the first subunit and can activate the target promoter in the second subunit, leading to the expression of the glycosylation-related genes, etc. of interest. Thus, when the glycosylation agent expression vector is constituted by two subunits, the glycosylation-related genes, etc. are to be under indirect expression control of the MSG or PSG promoter. The first and second subunits have the following configuration.

The “first subunit” comprises the MSG or PSG promoter and the transcriptional control element gene linked in an expressible state downstream of the promoter. In this respect, two or more transcriptional control element genes may be linked under the control of one MSG or PSG promoter. Examples thereof include GAL4 and tTA linked under the control of the MSG promoter. Alternatively, the first subunit may have two or more sets each consisting of the MSG or PSG promoter and the transcriptional control element gene under the control thereof. In this case, these sets may be the same as or different from each other. Examples thereof include a first subunit comprising a set consisting of the MSG promoter and the GAL4 gene, and a set consisting of the posterior silk gland promoter and the GAL4 gene.

A known MSG or PSG promoter can be used as the promoter contained in the first subunit. Therefore, an existing gene expression vector having the MSG or PSG promoter, prepared for silk-spinning insects such as silkworms can also be used.

The “second subunit” comprises the target promoter of the transcriptional control element encoded in the first subunit, and the glycosylation-related genes, etc. functionally linked downstream of the target promoter. The target promoter comprised in the second subunit is a promoter that is activated by the transcriptional control element encoded in the first subunit. Thus, the target promoter comprised in the second subunit is unambiguously determined by the transcriptional control element encoded in the first subunit, as a rule. For example, if the transcriptional control element gene comprised in the target promoter first subunit is GAL4 gene, UAS is used as the GAL4 target promoter in the second subunit. The second subunit may comprise two or more same or different glycosylation-related genes, etc. under the control of one target promoter. For example, a second subunit corresponds thereto which comprises three genes consisting of the GNE gene, the CMAS gene and the ST6GAL1 gene, four genes consisting of the GNE gene, the CMAS gene, the ST6GAL1 gene and the NANP gene, five genes consisting of the GNE gene, the CMAS gene, the ST6GAL1 gene, the NANS gene and the NANP gene, six genes consisting of the GNE gene, the CMAS gene, the ST6GAL1 gene, the NANS gene, the NANP gene and the SLC35A1 gene, or seven genes consisting of the GNE gene, the CMAS gene, the ST6GAL1 gene, the NANS gene, the NANP gene, the SLC35A1 gene, and the GalT gene, arranged under the control of one UAS.

Alternatively, the second subunit may have two or more sets each consisting of the target promoter and the glycosylation-related genes, etc. under the control thereof. In this case, these sets may be the same as or different from each other. For example, a second subunit corresponds thereto which comprises two UASs, six genes consisting of the GNE gene, the CMAS gene, the ST6GAL1 gene, the NANS gene, the NANP gene, and the SLC35A1 gene arranged under the control of one UAS, and the GalT gene arranged under the control of the other UAS.

The second subunit may be further constituted by two or more same or different units comprising the glycosylation-related genes, etc. In this case, the transcriptional control element expressed from one first subunit can activate the target promoters in a plurality of second subunits, leading to the expression of the glycosylation-related genes, etc. comprised in the respective second subunits. For example, second subunit A comprising six genes consisting of the GNE gene, the CMAS gene, the ST6GAL1 gene, the NANS gene, the NANP gene, and the SLC35A1 gene arranged under the control of UAS, and second subunit B comprising the GalT gene arranged under the control of UAS correspond thereto.

When the glycosylation agent expression vector is constituted by two subunits, a known or existing middle or posterior silk gland-specific gene expression vector can be used as the first subunit. Therefore, an existing transgenic silk-spinning insect line comprising such a gene expression vector can be used.

The glycosylation agent expression vector of this configuration can amplify the expression of the glycosylation-related genes, etc. in the second subunit via the transcriptional control element encoded in the first subunit. Thus, the glycosylation agent expression vector of this configuration is suitable for the overexpression of the glycosylation-related genes, etc. in host cells.

1-3-3. Configuration of Mammalian-Type Glycosylation Agent

The mammalian-type glycosylation agent of the present invention comprises one to three independent glycosylation agent expression vector(s). In the present specification, the term “independent” means that one glycosylation agent expression vector is capable of functioning by itself as one expression unit that can express at least one glycosylation-related gene, etc. Thus, when the glycosylation agent expression vector is constituted by two subunits as mentioned above, each subunit is not regarded as being “independent”, whereas a plurality of subunits together are interpreted as being “independent”. On the other hand, when the glycosylation agent expression vector is constituted by one unit, this unit can be interpreted as being “independent”.

When the mammalian-type glycosylation agent of the present invention is constituted by a plurality of independent glycosylation agent expression vectors, the combination of the glycosylation-related genes, etc. contained in the respective glycosylation agent expression vectors is not particularly limited. For example, a first glycosylation agent expression vector (first expression vector) may comprise only the GalT gene, and a second glycosylation agent expression vector (second expression vector) may comprise three or more sialic acid-related genes. Alternatively, the first expression vector may comprise only the GalT gene, the second expression vector may comprise three or more sialic acid-related genes except for the SLC35A1 gene, and a third glycosylation agent expression vector (third expression vector) may comprise only the SLC35A1 gene.

1-4. Introduction Method

A method for introducing the glycosylation agent expression vector into host cells by applying the mammalian-type glycosylation agent of the present aspect to the host will be described.

The host to which the glycosylation agent expression vector is to be introduced may be any of a silk-spinning insect individual, silk-spinning insect-derived cells (including an established cell line) and silk-spinning insect-derived tissues. The individual is not limited by its developmental stage. Any of the embryo, larva, pupa, and adult stages may be used. The embryo stage is preferred which can be expected to be highly effective. The host is not limited by its sex. Likewise, the cells or the tissues are not limited by the developmental stage of an individual from which the cells or the tissues are harvested or derived.

The introduction method can be performed by a method known in the art according to the status of introduction. For example, when the host used in the introduction is a silkworm and the exogenous gene expression vector is a plasmid having an inverted terminal repeat sequence of transposon (Handler A M. et al., 1998, Proc. Natl. Acad. Sci. U.S.A. 95: 7520-5), the introduction can be performed by use of the method of Tamura et al. (Tamura T. et al., 2000, Nature Biotechnology, 18, 81-84). Briefly, a helper vector having DNA encoding transposase can be injected together with the glycosylation agent expression vector to the early embryo of the silkworm. Examples of the helper vector include pHA3PIG. When the glycosylation agent expression vector of the present aspect comprises a marker gene, a transformant can be easily selected on the basis of the expression of the gene, etc. The transgenic silkworm obtained by this method has the glycosylation agent expression vector integrated in the chromosome via the inverted terminal repeat sequence of transposon. The obtained transgenic silkworm may be sib-mated or inbred, if necessary, to obtain a homozygote of the expression vector inserted in the chromosome.

### 2. Transgenic Silk-Spinning Insect Capable of Glycosylation with Mammalian-Type Sugar Chain

### 2-1. Summary

The second aspect of the present invention is a transgenic silk-spinning insect capable of glycosylation with a mammalian-type sugar chain. The transgenic silk-spinning insect capable of glycosylation with a mammalian-type sugar chain according to the present invention has the glycosylation agent expression vector of the first aspect and can attach a mammalian-type sugar chain to a recombinant protein produced in MSG and/or PSG.

### 2-2. Configuration

In the present specification, the “transgenic silk-spinning insect capable of glycosylation with a mammalian-type sugar chain (in the present specification, also referred to as a “glycosylation silk-spinning insect”)” refers to a transgenic silk-spinning insect having the glycosylation agent expression vector described in the first aspect. The silk-spinning insect serving as a host may be any of the silk-spinning insects mentioned above. A silkworm, Samia cynthia ricini and Antheraea pernyi are particularly preferred whose rearing method and artificial diet have been established and which can be reared at a large scale. When the host is a silkworm, the “transgenic silk-spinning insect capable of glycosylation with a mammalian-type sugar chain” according to the present aspect is referred to as a “transgenic silkworm capable of glycosylation with a mammalian-type sugar chain (in the present specification, also referred to as a “glycosylation silkworm”)”. Also, the mammalian-type sugar chain is preferably a human-type sugar chain.

The glycosylation silk-spinning insect of the present invention may transiently have the glycosylation agent expression vector of the first aspect in cells or may stably have the glycosylation agent expression vector of the first aspect, for example, in a state introduced in the genome. It is preferred to stably have the glycosylation agent expression vector of the first aspect.

The glycosylation silk-spinning insect of the present invention can have two or more different glycosylation agent expression vectors of the first aspect. For example, a glycosylation silk-spinning insect having the first expression vector and the second expression vector described in the first aspect corresponds thereto. When the glycosylation-related genes, etc. essential for achieving mammalian-type glycosylation are divided in the first expression vector and the second expression vector, a glycosylation silk-spinning insect having both the first expression vector and the second expression vector can exert the advantageous effects of the present invention. When the glycosylation agent expression vector is constituted by two subunits, the first subunit and the second subunit, a glycosylation silk-spinning insect having both the subunits can also exert the advantageous effects of the present invention.

When the glycosylation agent expression vector has two or three different expression vectors (first to third expression vectors), each of which is inserted in the chromosome of the transgenic silk-spinning insect, these expression vectors may reside on the same chromosome or may reside on different chromosomes. When the expression vectors reside on different chromosomes, the glycosylation silk-spinning insect of the present invention having the first expression vector and the second expression vector can be easily obtained as a first filial generation (F1) by mating a transgenic silk-spinning insect line having only the first expression vector with a transgenic silk-spinning insect line having only the second expression vector. On the other hand, when the first expression vector and the second expression vector reside on the same chromosome, it is preferred that the subunits should be located at a close interval and linked to each other so as not to be separated by recombination during the process of passage.

The same holds true for the case where the glycosylation agent expression vector is constituted by two subunits, the first and second subunits. For example, when the first and second subunits are located on different chromosomes, the glycosylation silk-spinning insect of the present invention having both the first and second subunits can be easily obtained as F1 by mating a transgenic silk-spinning insect line having only the first expression vector with a line producing a transgenic silk-spinning insect capable of glycosylation with a mammalian-type sugar chain mentioned later, having only the second expression vector.

### 3. Line Producing Transgenic Silk-Spinning Insect Capable of Glycosylation with a Mammalian-Type Sugar Chain

### 3-1. Summary

The third aspect of the present invention is a line producing a transgenic silk-spinning insect capable of glycosylation with a mammalian-type sugar chain. The line producing a transgenic silk-spinning insect according to the present invention is a transgenic silk-spinning insect having a portion of the glycosylation agent expression vector described in the first aspect, and progeny thereof. The glycosylation silk-spinning insect can be produced any time and easily by using this line.

### 3-2. Configuration

In the present specification, the “line producing a transgenic silk-spinning insect capable of glycosylation with a mammalian-type sugar chain (in the present specification, also referred to as a “glycosylation silk-spinning insect-producing line”)” refers to a transgenic silk-spinning insect that has the potential to attach a mammalian-type sugar chain to a protein produced in a silk gland and is capable of passage, or progeny thereof. The silk-spinning insect serving as a host may be any of the silk-spinning insects. For the same reason as in the glycosylation silk-spinning insect of the second aspect, a silkworm, Samia cynthia ricini and Antheraea pernyi are preferred. When the host is a silkworm, the “glycosylation silk-spinning insect-producing line” of the present aspect is referred to as a “glycosylation silkworm-producing line”. Also, the mammalian-type sugar chain is preferably a human-type sugar chain.

The glycosylation silk-spinning insect-producing line has a portion of the glycosylation agent expression vector described in the first aspect. When the glycosylation agent expression vector is constituted by two or three expression vectors, only one or some of the expression vectors corresponds to “a portion of the glycosylation agent expression vector”. When the glycosylation agent expression vector is constituted by two subunits, only the second subunit corresponds thereto. Specifically, the glycosylation silk-spinning incest-producing line has a portion of the glycosylation agent expression vector described in the first aspect and thereby has the potential to attach a mammalian-type sugar chain to a protein produced in a silk gland. However, the glycosylation silk-spinning worm-producing line does not comprise the minimum glycosylation-related genes, etc. necessary for glycosylation and therefore cannot attach a mammalian-type sugar chain to a protein solo. On the other hand, the glycosylation silk-spinning insect of interest can be easily produced, when needed, by ensuring the minimum necessary glycosylation-related genes, etc. within one individual through mating with another glycosylation silk-spinning insect-producing line or by inducing the expression of the minimum necessary glycosylation-related genes, etc. comprised in the second subunit through mating with a transgenic silk-spinning insect comprising the first subunit. The transgenic silk-spinning insect line having only the first subunit does not have the direct potential to attach a mammalian-type sugar chain and therefore does not correspond to the glycosylation silk-spinning worm-producing line of the present invention.

4. Method for producing transgenic silk-spinning insect capable of glycosylation with a mammalian-type sugar chain

### 4-1. Summary

The fourth aspect of the present invention is a method for producing a transgenic silk-spinning insect capable of glycosylation with a mammalian-type sugar chain (glycosylation silk-spinning insect). The method of the present invention can produce a glycosylation silk-spinning insect that can attach a mammalian-type sugar chain to the recombinant protein of interest expressed in a silk gland.

### 4-2. Method

The method for producing a transgenic silk-spinning insect according to the present invention comprises a mating step and a selection step. Hereinafter, each step will be described.

(1) Mating Step

The “mating step” is an essential step of mating the glycosylation silk-spinning worm-producing line having the second subunit described in the third aspect with a transgenic silk-spinning insect line having the first subunit. The silk-spinning insect serving as a host may be any of the silk-spinning insects. For the same reason as in the glycosylation silk-spinning insect of the second aspect, a silkworm, Samia cynthia ricini and Antheraea pernyi are preferred. The mating can be performed between the two silk-spinning insect lines described above on the basis of a routine method. The mating is performed between a male and a female of the same species, as a rule.

For the respective transgenic silk-spinning insects having the subunit inserted in the genome, it is preferred that the silk-spinning insects of the glycosylation silk-spinning worm-producing line and the transgenic silk-spinning insect line for use in mating should be sib-mated or inbred to obtain a homozygote of each subunit in advance. As a result, all first filial generation (F1) individuals are glycosylation silk-spinning insects having both the subunits. In this case, the next selection step is not an essential step and is an optional step that is performed, if necessary, for confirmation.

(2) Selection Step

The “selection step” is the step of selecting a transgenic silk-spinning insect comprising the two subunits as the glycosylation silk-spinning insect from F1 individuals. In this step, an individual having the activity of the marker proteins encoded in the respective subunits can be selected as the glycosylation silk-spinning insect of interest on the basis of the activity of these marker proteins from F1 individuals obtained after the mating step.

### EXAMPLES

Hereinafter, embodiments of the present invention will be described with reference to examples. However, the embodiments described herein are given merely for specifically illustrating the mode for carrying out the present invention. It should be understood that the scope of the present invention is not limited by the scope of Examples given below.

### Example 1: Construction of Each Expression Vector

(Purpose)

A glycosylation agent expression vector constituting the mammalian-type glycosylation agent of the present invention, and an expression vector for a recombinant protein of interest are constructed.

In Examples of the present specification, an expression vector consisting of two subunits, the first subunit and the second subunit, was adopted as each expression vector. Specifically, the expression vector used in Examples of the present specification was constituted by a first subunit comprising MSG promoter and the GAL4 gene functionally linked downstream of the promoter, and a second subunit comprising each sugar chain-related gene functionally linked downstream of UAS promoter, and a gene encoding each recombinant protein of interest.

(Method)

1. Construction of First Expression Unit

(1) Construction of First Subunit for Expression in Middle Silk Gland

pBacSer-pro GAL4/3×P3DsRed2 having a promoter of the sericin 1 gene specifically expressed in MSG, the transcriptional control element GAL4 gene functionally connected downstream of the promoter, and a hsp70 polyA addition sequence further connected downstream thereof was constructed as the first subunit inducing gene expression in the middle silk gland (MSG).

The sericin 1 gene promoter was prepared by PCR-amplifying a promoter-containing region, shown in SEQ ID NO: 1, corresponding to −666 to +40 (the transcription start site is defined as position 0; the same holds true for the description below) of the silkworm sericin 1 gene using genomic DNA of a silkworm Daizo line as a template and a primer pair consisting of an AscI site-containing primer shown in SEQ ID NO: 63 and a BamHI site-containing primer shown in SEQ ID NO: 64. The amplified fragment was inserted to pCR-Blunt II-TOPO vector (Thermo Fisher Scientific Inc.) and cleaved with AscI and BamHI. Then, the AscI-BamHI amplified fragment containing the promoter region was inserted to an AscI-BamHI site upstream of the GAL4 gene in pBacA3dGAL4 (Uchino K. et al., 2006, J Insect Biotechnol Sericol 75: 89-97). 3×P3-DsRed cassette excised from pBacA3GAL4/3×P3DsRed2 (Uchino K. et al., 2006, J Insect Biotechnol Sericol 75: 89-97) with BglII was inserted as a selection marker to the plasmid to construct a first subunit pBacSer-pro GAL4/3×P3DsRed2 for expression in MSG.

2. Construction of Second Expression Unit

(1) Construction of Basic Vector

PCR was performed using pBac[SerUAS-hr5/3×P3-EGFPinv] (Tada M. et al., 2015, MAbs. 7 (6): 1138-1150) as a template and a primer pair consisting of SerTATA-U (SEQ ID NO: 65) and BlnBsmSerK-L (SEQ ID NO: 66) to obtain an amplification product SnaBI-BsmBI fragment. Next, pBac[SerUAS_Ser1intron_hr5/3×P3-EYFP_A3-Bla] (Tada M. et al., 2015, MAbs. 7(6):1138-1150) was cleaved with SnaBI and BsmBI for the removal of a short fragment, and the SnaBI-BsmBI fragment was inserted to the SnaBI-BsmBI site. The resultant was designated as pBac[SerUAS_Ser1kozak_hr5/3×P3-EYFP_A3-Bla]. Subsequently, PCR was performed using pHC-EGFP as a template and a primer pair consisting of FibHsig-U (SEQ ID NO: 67) and FibHsig-L (SEQ ID NO: 68) to obtain an amplification product BspH-BlnI fragment. The pBac[SerUAS_Ser1kozak_hr5/3×P3-EYFP_A3-Bla] mentioned above was cleaved with BsmBI and BlnI for the removal of a short fragment, and the BspH-BlnI fragment was inserted to the BsmBI-BlnI site. The resultant was designated as pBac[SerUAS_FibHsigint_hr5/3×P3-EYFP_A3-Bla] and used as a basic expression vector for the second expression unit in an expression vector.

(2) Construction of Glycosylation Agent Expression Vector

A. Construction of GalT Expression Vector

(Construction of Conventional-Type GalT2 Expression Vector)

The promoter region and the 3′UTR region of the sericin 1 gene were each amplified using silkworm genomic DNA as a template. Then, both the amplified fragments were linked by overlap-extension PCR. In this operation, a BlnI site was inserted to the boundary between the promoter region and 3′UTR. This linked fragment was inserted to pTA vector (Toyobo Co., Ltd.), and the resultant was designated as pTA2[Ser-UTR]. A GAL4Δ fragment (Kobayashi I., et al., 2011, Arch Insect Biochem Physiol, 76: 195-210) was inserted to the BlnI site of pTA2[Ser-UTR], and the resultant was designated as pTA2[Ser-GAL4A]. The AscI fragment of pTA2[Ser-GAL4A] was inserted to the AscI site of pBac[A3KMO, UAS] (Kobayashi I., et al., 2007, J. Insect Biotechnol Sericol, 76: 145-48), and the resultant was designated as pBac[A3KMO, UAS, Ser-GAL4A].

The mouse-derived GalT2 open reading frame was amplified by PCR such that a BlnI site was added to the terminal. This fragment was inserted to the BlnI site of pBac[A3KMO, UAS, Ser-GAL4A], and the resultant was designated as pBac[SerUAS-GalT2p/A3-KMO_A3-Bla] which was a conventional-type GalT2 expression vector.

(Construction of Basic Vector for Improved-Type GalT Expression Vector)

The basic expression vector pBac[SerUAS_Ser1kozak_hr5/3×P3-EYFP_A3-Bla]constructed in the preceding section (1) was cleaved with AscI and XhoI for the removal of a short fragment, and an adapter prepared by annealing AscI-NheI-XhoI-U (SEQ ID NO: 69) and AscI-NheI-XhoI-L (SEQ ID NO: 70) was inserted to the AscI-XhoI site. The resultant was designated as pBac[SerUAS_Ser1kozak_hr5/NheIad_A3-Bla]. The NheI fragment of pBac[A3KMO, UAS, Ser-GAL4A] was inserted to the NheI site of the plasmid. The resultant was designated as pBac[SerUAS_Ser1kozak_hr5/A3-KMO_A3-Bla].

(Construction of Each Improved-Type GalT Expression Vector)

The mouse-derived GalT1 to GalT4 genes were subjected to PCR using primer pairs consisting of BspHI-mGalT1 U (SEQ ID NO: 71) and mouse BlnI-mGalT1 L (SEQ ID NO: 72) for mouse GalT1, BsmBI-GalT2-U (SEQ ID NO: 73) and BsmBI-GalT2-L (SEQ ID NO: 74) for GalT2, BsmBI-mGalT3 U (SEQ ID NO: 75) and mouse BsmBI-mGalT3 L (SEQ ID NO: 76) for mouse GalT3, and NcoI-mGalT4 U (SEQ ID NO: 77) and mouse BlnI-mGalT4 L (SEQ ID NO: 78) for mouse GalT4. Each amplification product was cleaved with a restriction enzyme described in each primer name and then inserted to the BsmBI site of the basic vector pBac[SerUAS_Ser1kozak_hr5/A3-KMO_A3-Bla] for an improved-type GalT vector. The obtained improved-type GalT expression vectors were designated as pBac[SerUAS-GalT1/A3-KMO_A3-Bla], pBac[SerUAS-GalT2i/A3-KMO_A3-Bla], pBac[SerUAS-GalT3/A3-KMO_A3-Bla], and pBac[SerUAS-GalT4/A3-KMO_A3-Bla], respectively. GalT2p represents conventional-type GalT2, and GalT2i represents improved-type GalT2.

B. Construction of Sialic Acid-Related Gene Expression Vector

(Construction of UAS Unit Vector)

PCR was performed using the basic expression vector pBac[SerUAS_Ser1kozak_hr5/3×P3-EYFP_A3-Bla] constructed in the preceding section (1) as a template and a primer pair consisting of serUASUNhe (SEQ ID NO: 79) and serPolyALSpe (SEQ ID NO: 80). The obtained amplification product was inserted to the EcoRV site of pZErO2 (Thermo Fisher Scientific Inc.). The resultant was designated as SerUAS_unit/pZErO2 which was a UAS unit vector.

(Insertion of Sialic Acid-Related Gene Fragment to UAS Unit Vector)

ORF of each of sialic acid-related genes (GNE, NANS, NANP, CMAS, ST6GAL1, and SLC35A1 genes) was amplified by PCR using a primer pair given below to obtain each sialic acid-related gene fragment. The primer pair used for the GNE gene was r2epiU (SEQ ID NO: 81) and r2epiL (SEQ ID NO: 82). The primer pair used for the NANS gene was BsmBI_NANS_U (SEQ ID NO: 83) and BsmBI_NANS_L (SEQ ID NO: 84). The primer pair used for the NANP gene was BsmBI_NANP_U (SEQ ID NO: 85) and BsmBI_NANP_L (SEQ ID NO: 86). The primer pair used for the CMAS gene was hCSSU (SEQ ID NO: 87) and hCSSL (SEQ ID NO: 88). The primer pair used for the SLC35A1 gene was BsmBI_hCST_U (SEQ ID NO: 89) and BsmBI_hCST_L (SEQ ID NO: 90). The primer pair used for the ST6GAL1 gene was hSTU (SEQ ID NO: 91) and hSTL (SEQ ID NO: 92). Each sialic acid-related gene fragment was cleaved with BsmBI and then inserted to the BsmBI site of the UAS unit vector SerUAS_unit/pZErO2 mentioned above. The finished UAS unit vectors comprising each sialic acid-related gene were designated as UAS-GNE/pZErO2 (for GNE expression), UAS-NANS/pZErO2 (for NANS expression), UAS-NANP/pZErO2 (for NANP expression), UAS-CMAS/pZErO2 (for CMAS expression), UAS-ST6GAL1/pZErO2 (for ST6GAL1 expression), and UAS-SLC35A1/pZErO2 (for SLC35A1 expression).

(Construction of HS4 Insulator Unit Vector)

A HS4 insulator sequence consisting of the nucleotide sequence represented by SEQ ID NO: 93 was synthesized on a consignment basis by GenScript Japan Inc., cleaved with NheI and SpeI, and inserted to the SpeI site of a vector containing the original HS4 insulator. This operation was repeated to construct a plasmid having 4 repeats of the HS4 insulator. This plasmid was designated as HS4×4/pUC.

(Construction of piggyBac/3×P3AmCyan Vector)

An adapter prepared by annealing SpeIadaptU (SEQ ID NO: 94) and SpeIadaptL (SEQ ID NO: 95) was inserted to the EcoRV-PstI site of pBac[SerUAS/3×P3EGFP] (Tatematsu K, et al., 2010, Transgenic Res. 19 (3): 473-87). The resultant was designated as pBac[3×P3EGFP]. The EcoRI fragment of pBac[SerUAS_Ser1intron_hr5/3×P3-AmCyan_A3-Bla] (Tada M. et al., 2015, MAbs. 7 (6): 1138-1150) was inserted to the EcoRI site of pBac[3×P3EGFP]. The resultant was designated as pBac[3×P3AmCyan].

(Insertion of UAS Sialic Acid Unit and HS4 Insulator to piggyBac/3×P3AmCyan)

The UAS unit vectors UAS-GNE/pZErO2 (for GNE expression), UAS-NANS/pZErO2 (for NANS expression), UAS-NANP/pZErO2 (for NANP expression), UAS-CMAS/pZErO2 (for CMAS expression), UAS-ST6GAL1/pZErO2 (for ST6GAL1 expression), and UAS-SLC35A1/pZErO2 (for SLC35A1 expression) comprising each sialic acid-related gene, and the HS4 insulator unit vector HS4×4/pUC mentioned above were each cleaved with NheI and SpeI. Subsequently, the NheI-SpeI fragments of 3 vectors (UAS-GNE/pZErO2, UAS-CMAS/pZErO2 and UAS-ST6GAL1/pZErO2) and the NheI-SpeI fragment containing the HS4 insulator were inserted to the SpeI site of piggyBac/3×P3AmCyan such that each UAS sialic acid unit and each repeat of the HS4 insulator were alternately linked. The resultant was designated as pBac[HS4-UAS-GNE-HS4-UAS-CMAS-HS4-UAS-ST6GAL1-HS4/3×P3AmCyan] (hereinafter, abbreviated to pBac[UAS-GNE/CAMS/ST6GAL1/3×P3AmCyan]). Next, the NheI-SpeI fragments of 2 vectors represented by UAS-NANS/pZErO2 and UAS-NANP/pZErO2 were inserted again to pBac[UAS-GNE/CAMS/ST6GAL1/3×P3AmCyan] such that these two NheI-SpeI fragments were sandwiched between the NheI-SpeI fragments containing the HS4 insulator. The resultant was designated as pBac[HS4-UAS-NANS-HS4-UAS-NANP-HS4-UAS-GNE-HS4-UAS-CMAS-HS4-UAS-ST6GAL1-HS4/3×P3AmCyan] (hereinafter, abbreviated to pBac[UAS-NANS/NANP/GNE/CAMS/ST6GAL1/3×P3AmCyan]). Also, the NheI-SpeI fragments of 3 vectors represented by UAS-NANS/pZErO2, UAS-NANP/pZErO2 and UAS-SLC35A1/pZErO2 were inserted to pBac[UAS-GNE/CAMS/ST6GAL1/3×P3AmCyan] such that these three NheI-SpeI fragments were sandwiched between the NheI-SpeI fragments containing the HS4 insulator. The resultant was designated as pBac[HS4-UAS-NANS-HS4-UAS-NANP-HS4-UAS-SLC35A1-HS4-UAS-GNE-HS4-UAS-CMAS-HS4-UAS-ST6GAL1-HS4/3×P3AmCyan] (hereinafter, abbreviated to pBac[UAS-NANS/NANP/SLC35A1/GNE/CAMS/ST6GAL1/3×P3AmCyan]). As a result, sialic acid-related gene expression vectors comprising three UAS sialic acid units (GNE/CAMS/ST6GAL1), five UAS sialic acid units (NANS/NANP/GNE/CAMS/ST6GAL1), or six UAS sialic acid units (NANS/NANP/SLC35A1/GNE/CAMS/ST6GAL1) were obtained.

(3) Construction of Expression Vector for Recombinant Protein of Interest

(Construction of Human Antithrombin III Expression Vector)

PCR was performed using vector Flexi ORF clone FHC11758 (Promega Corp.) containing ORF of the human antithrombin III (hATIII) gene as a template and a primer pair consisting of BsmBI_AT_FibHsig U40 (SEQ ID NO: 96) and AT_C-6His_L45 (SEQ ID NO: 97) containing a His tag. The BsmBI fragment of the obtained amplification product was inserted to the BsmBI site of the basic expression vector pBac[SerUAS_FibHsigint_hr5/3×P3-EYFP_A3-Bla] constructed in the section (1). The resultant was designated as UAS-rATIII/pZErO2.

(Construction of Human Interferon γ Expression Vector)

PCR was performed using Flexi ORF clone ORH24802 (Promega Corp.) containing ORF of the human interferon γ (hIFNγ) gene as a template and a primer pair consisting of hIFNg_FibHsigU40 (SEQ ID NO: 98) and hIFNg_C-6His_L45 (SEQ ID NO: 99) containing a His tag. The BsmBI fragment of the obtained amplification product was inserted to the BsmBI site of the basic expression vector pBac[SerUAS_FibHsigint_hr5/3×P3-EYFP_A3-Bla] constructed in the section (1). The resultant was designated as UAS-hIFNg/pZErO2.

### Example 2: Production of Transgenic Silkworm

(Purpose)

Various transgenic silkworms were produced using each expression vector constructed in Example 1.

(Material and Method)

(1) Silkworm Line

A w1-pnd line, which is a non-dormant line having white eyes and white eggs, maintained in National Institute of Agrobiological Sciences (NIAS) was used as a host line.

(2) Rearing Conditions

Larvae were reared with an artificial diet (SilkMate PS, Nosan Corp.) throughout the entire larval instars in a rearing room of 25 to 27° C. The artificial diet was replaced every 2 to 3 days (Uchino K. et al., 2006, J Insect Biotechnol Sericol, 75: 89-97).

(3) Production of Transgenic Silkworm

Transgenic silkworms were produced according to the method of Tamura et al. (Tamura T. et al., 2000, Nature Biotechnology, 18, 81-84). The first subunit and the second subunit constructed in Example 1 were each separately mixed with a helper plasmid pHA3PIG (Tamura T. et al., 2000, Nature Biotechnology, 18, 81-84) expressing transposase at a ratio of 1:1, and the mixture was injected to silkworm eggs 2 to 8 hours after egg laying. The eggs after the injection were incubated at 25° C. in a humidified state until hatching. Hatched larvae were reared by the method described above.

(Production of Transgenic Silkworm Line for GAL4 Expression)

For the production of a transgenic silkworm line for GAL4 expression comprising the first subunit, the first subunit pBacSer-pro GAL4/3×P3DsRed2 for MSG expression was used in the injection described above. Hatched larvae after the injection were reared by the method described above. F1 individuals comprising the first subunit were selected on the basis of the presence or absence of eye fluorescence by the 3×P3DsRed2 marker to obtain a transgenic silkworm line for GAL4 expression comprising the first subunit of the transgenic silkworm of the present invention.

(Production of GalT Glycosylation Silkworm Producing Line)

For the production of a GalT glycosylation silkworm-producing line comprising the second subunit, the GalT2p expression vector and each improved-type GalT expression vector (pBac[SerUAS-GalT1/A3-KMO_A3-Bla], pBac[SerUAS-GalT2i/A3-KMO_A3-Bla], pBac[SerUAS-GalT3/A3-KMO_A3-Bla], and pBac[SerUAS-GalT4/A3-KMO_A3-Bla]) were each used in the injection described above. Hatched larvae after the injection were reared by the method described above. F1 individuals comprising each improved-type GalT expression vector were selected on the basis of the colored body surface of the first instar larvae by the A3-KMO marker to obtain each GalT glycosylation silkworm-producing line.

(Production of Sialic Acid-Related Protein Glycosylation Silkworm-Producing Line)

For the production of a sialic acid-related protein glycosylation silkworm-producing line comprising the second subunit, each sialic acid-related gene expression vector mentioned above was used in the injection described above. Hatched larvae after the injection were reared by the method described above. F1 individuals comprising each expression vector were selected on the basis of the presence or absence of eye fluorescence by the 3×P3-AmCyan marker to obtain each sialic acid-related protein glycosylation silkworm-producing line.

(Production of Transgenic Silkworm Line for Expression of Recombinant Protein of Interest)

For the production of a transgenic silkworm line for the expression of the recombinant protein of interest comprising the second subunit, the antithrombin III expression vector or the interferon γ expression vector mentioned above was used in the injection mentioned above. Hatched larvae after the injection were reared by the method described above. F1 individuals comprising each expression vector were selected on the basis of the presence or absence of eye fluorescence by the 3×P3-EYFP marker to obtain a transgenic silkworm line for ATIII expression and a transgenic silkworm line for INFγ expression. Then, the F1 individuals were sib-mated to obtain homozygotes.

(4) Mating of Lines Comprising First and Second Subunits

The transgenic silkworm line for GAL4 expression comprising the first subunit was mated with each line comprising the second subunit (GalT glycosylation silkworm-producing line, each sialic acid-related protein glycosylation silkworm-producing line, and transgenic silkworm line for the expression of the recombinant protein of interest).

Specifically, first, the transgenic silkworm line for GAL4 expression comprising the first subunit was mated with the transgenic silkworm line for the expression of the recombinant protein of interest (ATIII or IFNγ) comprising the second subunit; the transgenic silkworm line for GAL4 expression comprising the first subunit was mated with the GalT glycosylation silkworm-producing line comprising the second subunit; and the transgenic silkworm line for GAL4 expression comprising the first subunit was mated with each sialic acid-related protein glycosylation silkworm-producing line comprising the second subunit. The expression of each gene was confirmed, and protein extraction was performed. Each first filial generation (F1) comprising the first and second subunits was selected. The [GAL4×ATIII or IFNγ] F1 silkworm was mated with the [GAL4×GalT] F1 silkworm; and the [GAL4×ATIII or IFNγ] F1 silkworm was mated with the [GAL4×sialic acid-related proteins] F1 silkworm. The expression of each gene was confirmed, and protein extraction was performed. Each second filial generation (F2) comprising the first subunit and the two second subunits was selected. Finally, the [GAL4×ATIII or IFNγ/GalT] F2 silkworm was mated with the [GAL4×ATIII or IFNγ/sialic acid-related proteins] F2 silkworm. The expression of each gene was confirmed, and protein extraction was performed. As a result, a F3 silkworm comprising the four subunits (first subunit and three second subunits) was produced. In addition, the [GAL4×GalT] F1 silkworm was mated with [GAL4×sialic acid silkworm] F1 silkworm by the same procedures as above to produce a [GAL4×GalT/sialic acid-related proteins] F2 silkworm. Since the transgenic silkworm line for GAL4 expression, each sialic acid-related gene transgenic silkworm line, each GalT gene transgenic silkworm line, and each transgenic silkworm for the expression of the recombinant protein of interest all differed in selection marker, the order of mating is not particularly limited.

Table 1 shows the combinations of the transgenic silkworm comprising the GalT expression vector (GalT gene transgenic silkworm) and the transgenic silkworms comprising various sialic acid-related gene expression vectors (sialic acid-related gene transgenic silkworms). The respective lines having the subunits were selected from F1 individuals after mating on the basis of the marker.

TABLE 1

| | | Galt gene transgenic silkworm | | | | | |
|---|---|---|---|---|---|---|---|
| | | — | GalT1 | GalT2p | GalT2i | GalT3 | GalT4 |
| Sialic acid- | — | ∘ | ∘ | ∘ | ∘ | ∘ | ∘ |
| related gene | 3 genes | ∘ | x | ∘ | ∘ | x | x |
| transgenic | 5 genes | ∘ | ∘ | ∘ | ∘ | x | x |
| silkworm | 6 genes | ∘ | ∘ | ∘ | ∘ | x | x |

3 genes: rGNE, hCAMS, and hST6GAL1
5 genes: rGNE, hCAMS, hST6GAL1, hNANS, and hNANP
6 genes: rGNE, hCAMS, hST6GAL1, hNANS, hNANP, and SLC35A1

In the table, “-” represents that the transgenic silkworm comprising the GalT expression vector or the sialic acid-related gene expression vector was not used in mating. In the table, the circle represents that each transgenic silkworm having the expression vectors was obtained by mating. The x-mark represents that mating was not performed.

In this Example, transgenic silkworms were obtained which comprised pBacSer-pro GAL4/3×P3DsRed2 as the common first subunit for expression in MSG as well as only the expression vector for the recombinant protein of interest (ATIII or IFNγ), only the GalT gene expression vector, only the sialic acid-related gene expression vector (3 genes, 5 genes, or 6 genes), recombinant protein-of-interest expression vector/GalT gene expression vector, recombinant protein-of-interest expression vector/sialic acid-related gene expression vector (3 genes, 5 genes, or 6 genes), GalT gene expression vector/sialic acid-related gene expression vector (3 genes, 5 genes, or 6 genes), or recombinant protein-of-interest expression vector/GalT gene expression vector/sialic acid-related gene expression vector as the second subunit(s).

### Example 3: Extraction of MSG Lumen Protein of Glycosylation Silkworm

(Purpose)

Lumen proteins including the recombinant protein are extracted from the MSG lumens of the ATIII glycosylation silkworms, the IFNγ glycosylation silkworms, and the non-ATIII/IFNγ-expressing silkworms for control produced in Example 2.

(Method)

Each glycosylation silkworm produced in Example 2 was reared in the same way as in Example 2. The fifth instar day-6 larva was anesthetized on ice immediately before spinning. An incision was made on its back, and MSG was excised using tweezers without damage (see Yasushi Mori, ed., Kaiko ni yoru shin seibutsugaku jikken (New Biological Experiments using Silkworms in English), Sanseido Bookstore Ltd., 1970, pp. 249-255). Subsequently, the excised MSG was fixed with ethanol and separated into lumen proteins and cells using tweezers. The lumen proteins obtained from each of the ATIII glycosylation silkworms and the IFNγ glycosylation silkworms were dissolved in LiBr to prepare a MSG extract. Alternatively, the excised MSG was placed in 1 mL of a 100 mM phosphate buffer (pH 7.2) per gland and shaken at 4° C. for 2 hours to extract water-soluble proteins. Then, the extract was centrifuged at 2000×g for 10 minutes, followed by the recovery of a supernatant.

### Example 4: Purification and Preparation of Recombinant Protein from MSG Extract

(Purpose)

The recombinant protein of interest obtained in Example 3 is separated and purified, and prepared for sugar chain structure analysis.

(Method)

(1) Measurement of Protein Concentration

A protein concentration in each MSG extract was measured by the Bradford method. BSA was used as a standard protein. The absorbance at OD₅₉₅ was measured as to serial dilutions (BSA concentration: 0 to 1.0 mg/mL) prepared using a Bradford reagent (Nacalai Tesque, Inc.) to prepare a calibration curve. OD₅₉₅ was also measured as to each MSG extract, and the protein concentration was determined by comparison with the calibration curve.

(2) Protein Purification

(Human Antithrombin III Purification)

A column was packed with 500 μL of Ni resin (profanity IMAC Ni-charged resin) and equilibrated with an equilibrating solution (50 mM sodium phosphate buffer/0.3 M NaCl). Five hundreds (500) μL of the MSG extract was loaded in the column, which was then washed with 1.5 mL of a washing solution (50 mM sodium phosphate buffer/0.3 M NaCl/10 mM imidazole). Subsequently, the protein of interest was eluted with 300 μL of an eluent (50 mM sodium phosphate buffer/0.3 M NaCl/200 mM imidazole). The eluate was concentrated with a 50 mM sodium phosphate buffer using Amicon Ultra-0.5 mL (30K), and the protein concentration was measured. Then, 4× sample buffer (4.0 mL of glycerol/1.67 mL of 1.5 M Tris-HCl buffer (pH 6.8)/1.0 mL of 10% SDS solution/400 μL of (3-mercaptoethanol) was added to a 1 μg aliquot, and the protein was denatured at 100° C. for 3 minutes and separated by SDS-PAGE.

(Human Interferon γ Purification)

A column was packed with 50 μL of Ni resin (profanity IMAC Ni-charged resin) and equilibrated with an equilibrating solution (50 mM sodium phosphate buffer/0.3 M NaCl). Subsequently, 500 μL of the MSG extract was loaded in the column, which was then washed with 1.5 mL of a washing solution (50 mM sodium phosphate buffer/0.3 M NaCl/20 mM imidazole). Subsequently, the protein of interest was eluted with 300 μL of an eluent (50 mM sodium phosphate buffer/0.3 M NaCl/200 mM imidazole). The eluate was concentrated with a 50 mM sodium phosphate buffer using Amicon Ultra-0.5 mL (10K), and the protein concentration was measured. Then, 4× sample buffer (4.0 mL of glycerol/1.67 mL of 1.5 M Tris-HCl buffer (pH 6.8)/1.0 mL of 10% SDS solution/400 μL of (3-mercaptoethanol) was added to a 1 μg aliquot, and the protein was denatured at 100° C. for 3 minutes and separated by SDS-PAGE.

(3) Protein Separation

(Human Antithrombin III Sample Preparation)

Three point three (3.3) μL of 4× sample buffer (4.0 mL of glycerol/1.67 mL of 1.5 M Tris-HCl buffer (pH 6.8)/1.0 mL of 10% SDS solution/400 μL of β-mercaptoethanol) was added to 10 μL of each eluate from the Ni column, and the eluate was denatured at 100° C. for 3 minutes. After centrifugation, the supernatant was recovered, and 1.1 μL of the MSG extract and 10 μL of the recovered supernatant were subjected to SDS-PAGE separation. The gel after the electrophoresis was stained with CBB to confirm hATIII purification.

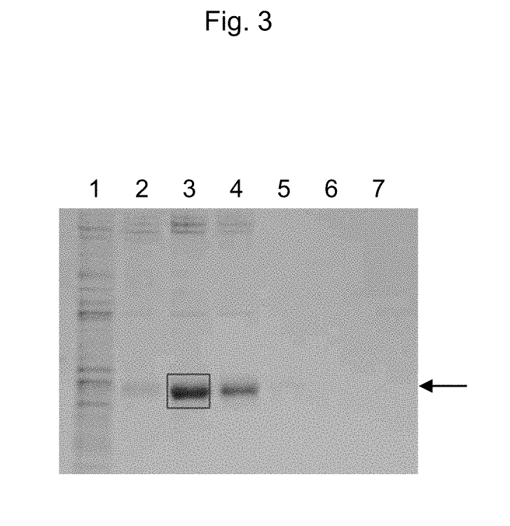

The results are shown in FIG. 3. The band of hATIII was able to be confirmed in the purified fraction.

(Human Interferon γ Sample Preparation)

Each eluate from the Ni column was adjusted to 1.2 mL with acetone and left standing at −20° C. for 3 days. Then, the supernatant was removed, followed by centrifugal drying. The precipitate was dissolved by the addition of 20 μL of 1× sample buffer and denatured at 100° C. for 3 minutes. After centrifugation, the supernatant was recovered, and 11.25 μL of the MSG extract and 10 μL of the recovered supernatant were subjected to SDS-PAGE separation. The gel after the electrophoresis was stained with CBB to confirm hIFNγ purification.

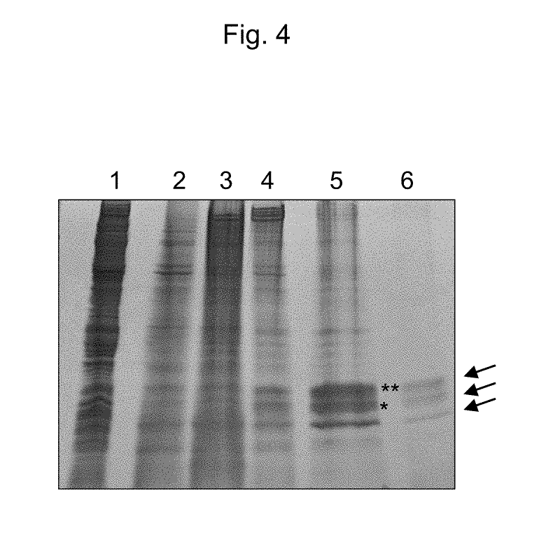

The results are shown in FIG. 4. Since three bands were confirmed in the purified fraction, hIFNγ was detected by Western blotting using a mouse-derived anti-His antibody diluted 3000-fold. The hIFNγ gene fragment contained the 6-His tag at the terminal by the primers used for cloning, as mentioned above. A HRP anti-mouse IgG antibody diluted 10000-fold was used as a secondary antibody. As a result, the three bands were all confirmed to be hIFNγ. hIFNγ deglycosylated using PNGase F was detected by Western blotting. As a result, the three bands converged to one band at the smallest molecular weight. From these results, it was predicted that three divided bands were detected depending on the presence or absence of glycosylation at two glycosylation sites of hIFNγ.

(4) In-Gel Digestion

(In-Gel Digestion of Human Interferon γ)

The protein (hIFNγ) bands (two bands of sugar chain-attached proteins indicated by * and ** in FIG. 4) separated by SDS-PAGE were excised and destained by 15-minute vortex twice using 50 mM NH₄HCO₃/50% MeCN (acetonitrile). Then, the resulting gel was vortexed with 100% MeCN for 5 minutes and vortexed again overnight with 50 mM NH4HCO3/50% MeCN. On the next morning, 15-minute vortex was performed twice using 100% MeCN, and MeCN was removed by centrifugal drying.

(Intra-Gel Digestion of Human Antithrombin III)

The protein (hATIII) band of interest separated by SDS-PAGE was excised and destained by 15-minute vortex twice using 50 mM NH4HCO3/50% MeCN (acetonitrile). Then, the resulting gel was vortexed with 100% MeCN for 5 minutes and vortexed again overnight with 50 mM NH4HCO3/50% MeCN. On the next morning, 15-minute vortex was performed twice using 100% MeCN, and MeCN was removed by centrifugal drying.

Reduction reaction was performed at 56° C. for 20 minutes by the addition of 50 mM NH₄HCO₃ containing 25 mM DDT. Then, the DTT solution as the supernatant was removed, and the same amount thereas of 50 mM NH4HCO3 containing 50 mM iodoacetamide was added to the gel, followed by occasional shaking for 20 minutes in the dark. Purified water was added thereto, and the mixture was vortexed. Then, MeCN was added thereto, and 10-minute vortex was repeated twice. 50 mM NH4HCO3 was added thereto, and the gel was swollen for 15 minutes and then dehydrated with MeCN, followed by the removal of MeCN by centrifugal drying.

20 μL of a trypsin solution was added thereto on ice, and the gel was left standing for 30 minutes and swollen. Then, 50 mM NH4HCO3 containing 0.01% ProteaseMAX™ (Promega Corp.) was added thereto so as to soak the gel, followed by incubation at 50° C. for 1 hour. The protease treatment may be performed using chymotrypsin. In this case, the incubation is performed at 25° C. for 2 to 18 hours. The enzymatically treated solution was recovered by centrifugation at 15000×g for 10 minutes. In order to inactivate trypsin, TFA was added thereto at 0.5%.

### Example 5: Sugar Chain Structure Analysis of Recombinant Protein

(Purpose)

The structures of sugar chains attached to the digested peptides obtained in Example 4 are analyzed by use of nanoLC-MS/MS.

(Method)

6 μL or 8 μL of the enzymatically treated solution obtained in Example 4 was subjected to nanoLC-MS/MS under analysis conditions given below. A specific analysis method abided by the instruction manual attached to each instrument used.

(Liquid Chromatography (LC) Conditions)
Instrument used: Agilent Technologies 1200 series
Eluent A: 0.1% HCOOH/Milli-Q
Eluent B: 0.1% HCOOH/MeCN
Column: ZORBAX 300SB-C18 (Agilent Technologies, Inc.) 150 mm×100 μm, 3.5 μm particles
Flow rate: 0.6 μL/min
Time schedule concentration gradient (min): 0→5→65→66→71→72→90
   B (%): 2→8→50→95→95→2→2

(Ms/Ms Conditions)
Instrument used: micrOTOF-Q Bruker
Mass range: 50 to 4,500 m/z
Ionization method: ESI
Scan rate: 5 KHz
Analysis software: Hystar

Sugar chain-attached peptides were identified from the results of nanoLC-MS/MS, and the structures of the sugar chains attached to the peptides were predicted.

(Results)

(1) Sugar Chain Structure Analysis of Human Antithrombin III

The human antithrombin III is known to have 4 asparagine residues (N128, N167, N87, and N224; the initiation methionine is defined as Ml; the same holds true for the description below) that undergo glycosylation (Zhou Z. & Smith D. L., 1990, Biomedical and environmental mass spectrometry, 19: 782-786).

(Comparison of Galactose Attachment Efficiency Among GalT Isozymes)

The sugar chain structures of proteins prepared from the silk glands of the transgenic silkworms harboring each isozyme gene of GalT were analyzed. Table 2 shows the profiles of the sugar chain structures. In the table, the numeric values indicate the ratio (%) of each sugar chain with respect to 100% in total of sugar chains detected.

TABLE 2

| Sugar chain structure | GalT2p | GalT2i | GalT1 | GalT3 | GalT4 |
|---|---|---|---|---|---|
| Man₅A | 17.82 | 12.75 | 24.93 | 12.54 | 31.33 |
| Man₃ | 8.40 | 6.39 | 11.31 | 7.93 | 12.01 |
| Man₃F | | | | 5.83 | |
| Man₂F | 9.69 | 27.84 | 16.47 | 7.56 | 8.10 |
| _{GlcNAc}Man₃ | 18.12 | 15.42 | 5.83 | 10.92 | 7.13 |
| ^{GlcNAc}Man₃ | 3.69 | 4.72 | | 4.83 | |
| _{GlcNAc}Man₃F | | | | 4.06 | 0.78 |
| ^{GlcNAc}Man₃F | | | | 1.23 | |
| ^{GlcNAc}Man₂B | | | | 0.94 | |
| GlcNAc₂Man₃ | 0.94 | 1.04 | 8.37 | 25.66 | 16.31 |
| GlcNAc₂Man₃F | | | | 1.44 | 0.59 |
| _{Gal}GlcNAcMan₃ | 3.99 | 3.77 | — | 1.48 | — |
| ^{Gal}GlcNAcMan₃ | | | | 1.02 | |
| _{Gal}GlcNAcMan₃F | 0.81 | — | — | 1.54 | — |
| GalGlcNAc₂Man₃ | 0.63 | 2.53 | 1.42 | 1.60 | 0.74 |
| Gal₂GlcNAc₂Man₃ | 0.51 | 1.13 | 0.95 | 0.74 | — |
| GalGlcNAcMan₅A | 10.38 | 2.06 | 5.12 | 1.11 | — |
| Total Gal attachment | 16.32 | 9.49 | 7.49 | 7.49 | 0.74 |

As seen from the results of Table 2, among the GalT isozymes, GalT2p (conventional-type GalT2) had the highest galactose attachment efficiency, followed by GalT2i (improved-type GalT2). It was further revealed that: GalT1 most generally used for galactose attachment in the previous studies has the same level of attachment efficiency as that of GalT3; and the attachment efficiency of GalT4 is lowest. Accordingly, data obtained using the GalT2p, GalT2i, and GalT1 gene transgenic silkworms will be shown below.

(Relationship Between the Number of Sialic Acid-Related Gene Introduced and Sugar Chain Structure)

In order to test the number of sialic acid-related genes introduced and the presence or absence of sialic acid attachment, the sugar chain structures of proteins prepared from the silk glands of the respective glycosylation silkworms obtained by mating a GalT gene transgenic silkworm with various sialic acid-related gene transgenic silkworms (3 genes, 5 genes, and 6 genes) were analyzed. Table 3 shows the profiles of the sugar chain structures. The gene of GalT2i found to have high galactose attachment efficiency was used as the GalT gene. The results are shown in Table 3. In the table, the numeric values indicate the ratio (%) of each sugar chain with respect to 100% in total of sugar chains detected. In the table, for example, Man₅ represents a structure containing 5 mannoses at a sugar chain terminal, and Man₃F represents a structure containing 3 mannoses and 1 fucose modification.

TABLE 3

| | Galactose | | | |
|---|---|---|---|---|
| | GalT2i | | | |
| Sialic acid | — | 3 genes | 5 genes | 6 genes |
| Man₅ | 12.75 | 28.29 | 11.15 | 15.64 |
| Man₄ | 1.34 | 2.46 | 8.45 | 0 |
| Man₃ | 6.39 | 8.83 | 5.65 | 4.69 |
| Man₃F | 1.46 | 0 | 0 | 0 |
| Man₂ | 5.02 | 4.24 | 1.03 | 2.66 |
| Man₂F | 27.84 | 14.11 | 2.33 | 2.70 |
| _{GlcNAc}Man₃ | 15.42 | 0 | 8.19 | 11.32 |
| ^{GlcNAc}Man₃ | 4.72 | 4.16 | 0.86 | 0 |
| _{GlcNAc}Man₃F | 0 | 0.96 | 0.77 | 0 |
| GlcNAc₂Man₃ | 1.04 | 13.26 | 12.04 | 6.66 |
| _{Gal}GlcNAcMan₃ | 3.77 | 3.61 | 9.85 | 0 |
| _{Gal}GlcNAcMan₃F | 0 | 0 | 0 | 0 |
| GalGlcNAc₂Man₃ | 2.53 | 2.18 | 1.57 | 0 |
| Gal₂GlcNAc₂Man₃ | 1.13 | 0 | 0.55 | 0 |
| GalGlcNAcMan₅ | 2.06 | 0 | 3.46 | 0 |
| _{Sia}GalGlcNAcMan₃ | 0 | 1.54 | 2.17 | 19.91 |
| Sia₂Gal₂GlcNAc₂Man₃ | 0 | 0 | 0 | 1.00 |
| SiaGalGlcNAcMan₅ | 0 | 0 | 2.47 | 18.34 |

The glycoproteins from the sialic acid-related gene transgenic silkworms comprising 3, 5 or 6 sialic acid-related genes were confirmed to have a sialic acid-terminated structure. These results revealed that at least three genes selected from the GNE, NANS, NANP, CMAS, and ST6GAL1 genes suffice as the number of sialic acid-related genes introduced necessary for attaching sialic acid to a galactose non-reducing terminal. Sialic acid attachment efficiency was significantly increased for the 6 genes compared with those of 3 genes or the 5 genes. The sialic acid-related gene that differs between the sialic acid-related gene expression vector of 5 genes (NANS/NANP/GNE/CAMS/ST6GAL1) and the sialic acid-related gene expression vector of 6 genes (NANS/NANP/SLC35A1/GNE/CAMS/ST6GAL1) prepared in Example 1 is the SLC35A1 gene. Thus, it was revealed that the SLC35A1 gene is not essential for attaching sialic acid to a galactose non-reducing terminal, but has a function of enhancing the attachment efficiency.

(Analysis of Sugar Chain Structure at Glycosylation Site)

Table 4 shows the relationship between sugar chain structures at two glycosylation sites (N187 and N224) of recombinant hATIII protein produced by glycosylation silkworms obtained by mating the GalT gene transgenic silkworm (GalT1, GalT2p, or GalT2i), the sialic acid-related gene transgenic silkworm (5 genes or 6 genes), and the hATIII gene transgenic silkworm. In the table, Mano-4 represents a structure containing 0 to 4 mannoses at a sugar chain terminal, and Man₂₋₃F represents a structure containing 2 to 3 mannoses and 1 fucose modification. Sugar chains having GlcNAc at a non-reducing terminal are collectively shown as a GlcNAc structure. Sugar chains having Gal at a non-reducing terminal are collectively shown as a Gal structure. Sugar chains having Sia at a non-reducing terminal are collectively shown as a Sia structure. a and b represent silkworm lines. In the table, the numeric values indicate the ratio (%) of each sugar chain with respect to 100% in total of sugar chains detected. As for the notation of ATIII/GalT/sialic acid-related genes in the table, (+/−/−) represents the hATIII gene transgenic silkworm, and (+/−/5 genes) represents a transgenic silkworm obtained by mating the hATIII gene transgenic silkworm with the sialic acid-related gene transgenic silkworm of 5 genes without mating the GalT gene transgenic silkworm. Likewise, (+/GalT2p/−) represents a transgenic silkworm obtained by mating the hATIII gene transgenic silkworm with the GalT2p gene transgenic silkworm without mating the sialic acid-related gene transgenic silkworm. (+/GalT2p/5 genes) represents a transgenic silkworm obtained by mating the hATIII gene transgenic silkworm, the GalT2p gene transgenic silkworm and the sialic acid-related gene transgenic silkworm of 5 genes. The same holds true for the other notation.

TABLE 4

| | | ATIII | | | | | | | | | |
|---|---|---|---|---|---|---|---|---|---|---|---|
| | | + | | | | | | | | | |
| | | GaIT | | | | | | | | | |
| | | − | | | GalT2p | | | | GalT2i | GalT1 | |
| | | Sialic acid-related gene | | | | | | | | | |
| | | | 5 | 6 | | 5 | 6 genes | | 6 | | 6 |
| | | − | genes | genes | − | genes | a | b | genes | − | genes |
| N187 | Man₀₋₄ | 30.5 | 32.0 | 32.0 | 20.4 | 27.5 | 3.3 | 16.2 | 16.7 | 24.7 | 16.8 |
| | Man₅ | 24.2 | 25.7 | 24.5 | 24.2 | 20.0 | 27.8 | 25.0 | 24.2 | 28.6 | 28.7 |
| | Man₈₋₉ | 33.4 | 32.0 | 35.3 | 41.4 | 31.2 | 47.5 | 48.7 | 47.1 | 31.0 | 37.6 |
| | Man₂₋₃F | 0 | 0 | 0 | 0 | 0 | 0 | 0 | 0 | 0 | 0 |
| | GlcNAc | 11.9 | 10.5 | 8.3 | 14.0 | 14.7 | 8.5 | 4.5 | 8.1 | 15.7 | 4.5 |
| | Gal | 0 | 0 | 0 | 0 | 0.9 | 2.7 | 2.0 | 0 | 0 | 0 |
| | Sia | 0 | 0 | 0 | 0 | 5.8 | 10.1 | 3.5 | 3.9 | 0 | 12.3 |
| N224 | Man₀₋₄ | 64.3 | 72.5 | 73.3 | 28.5 | 30.4 | 7.3 | 21.5 | 26.5 | 25.4 | 22.6 |
| | Man₅ | 4.3 | 2.8 | 3.3 | 11.1 | 13.0 | 13.6 | 7.3 | 20.0 | 6.9 | 9.6 |
| | Man₆₋₉ | 8.0 | 1.8 | 1.5 | 6.0 | 3.2 | 9.4 | 3.7 | 6.6 | 0 | 0 |
| | Man₂₋₃F | 7.5 | 4.2 | 9.4 | 0 | 1.0 | 0.3 | 0 | 0 | 0 | 0 |
| | GlcNAc | 15.9 | 18.7 | 12.3 | 36.3 | 27.0 | 21.9 | 19.2 | 27.7 | 29.8 | 17.5 |
| | Gal | 0 | 0 | 0 | 18.0 | 18.1 | 19.9 | 26.7 | 12.6 | 37.7 | 18.0 |
| | Sia | 0 | 0 | 0 | 0 | 7.3 | 29.5 | 22.7 | 6.8 | 0 | 32.4 |

Both the sugar chain-attached asparagine residues N187 and N224 had sialic acid attached to a non-reducing terminal, as in mammals, only in the transgenic silkworms comprising the GalT (GalT2p, GalT2i or GalT1) expression vector and the sialic acid-related gene expression vector of 5 genes or 6 genes. The efficiency was higher when GalT2p or GalT2i was used than when GalT1 was used.

(2) Sugar Chain Structure Analysis of Human Interferon γ

For human interferon γ (hIFNγ), the central band indicated by * in FIG. 4 was designated as a first molecular species, and the band with the slowest mobility indicated by ** was designated as a second molecular species.

The human interferon γ is known to have two asparagine residues (N48 and N120) that undergo glycosylation. Table 5 shows sugar chain structures at the glycosylation sites N48 and N120 in the first and second molecular species of recombinant hIFNγ protein obtained from glycosylation silkworms obtained by mating the GalT2p gene transgenic silkworm, the sialic acid-related gene transgenic silkworm (5 genes or 6 genes), and the hIFNγ gene transgenic silkworm. In the table, the numeric values indicate the ratio (%) of each sugar chain with respect to 100% in total of sugar chains detected. a and b represent silkworm lines. As for the notation of IFNγ/GalT2p/sialic acid-related genes in the table, (+/−/−) represents the hIFNγ gene transgenic silkworm, and (+/−/5 genes) represents a transgenic silkworm obtained by mating the hIFNγ gene transgenic silkworm with the sialic acid-related gene transgenic silkworm of 5 genes without mating the GalT2p gene transgenic silkworm. Likewise, (+/+/−) represents a transgenic silkworm obtained by mating the hIFNγ gene transgenic silkworm with the GalT2p gene transgenic silkworm without mating the sialic acid-related gene transgenic silkworm. (+/+/5 genes or 6 genes) represents a transgenic silkworm obtained by mating the GalT2p gene transgenic silkworm, the hIFNγ gene transgenic silkworm, and the sialic acid-related gene transgenic silkworm of 5 genes or 6 genes.

Table 6 shows excerpts of the sugar chain structures directly related to the present invention and the ratios (%) thereof from Table 5.

Table 7 shows top 4 abundance ratios of the sugar chain structures detected in each molecular species. The transgenic silkworm for control in this table is a hIFNγ gene transgenic silkworm obtained without mating the GalT gene transgenic silkworm and the sialic acid-related gene transgenic silkworm. Thus, in the transgenic silkworm for control, substantially a wild-type silkworm sugar chain was attached.

TABLE 5

| | | IFNγ | | | | | | | | | |
|---|---|---|---|---|---|---|---|---|---|---|---|
| | | + | | | | | | | | | |
| | | GalT2p | | | | | | | | | |
| | | − | | | | + | | | | | |
| | | Sialic acid-related gene | | | | | | | | | |
| | | − | | 5 genes | | − | | 5 genes | | 6 genes | |
| | | a | b | a | b | a | b | a | b | a | b |
| N48 | Man₀₋₄ | 39.5 | 44.1 | 36.6 | 24.5 | 20.6 | 12.1 | 18.2 | 8.7 | 9.4 | 3.9 |
| | Man₅ | 0 | 0 | 0 | 0 | 5.9 | 5.9 | 10.7 | 15.3 | 18.0 | 13.0 |
| | Man₈₋₉ | 0 | 0 | 0 | 0 | 0 | 0 | 0 | 0 | 0 | 0 |
| | Man₂₋₃F | 16.8 | 15.4 | 19.6 | 14.8 | 0 | 1.3 | 1.4 | 1.0 | 1.8 | 0 |
| | GlcNAc | 43.6 | 40.5 | 44.0 | 60.8 | 42.9 | 42.2 | 17.5 | 20.0 | 16.6 | 7.5 |
| | Gal | 0 | 0 | 0 | 0 | 30.7 | 38.5 | 5.7 | 9.2 | 7.4 | 11.1 |
| | Sia | 0 | 0 | 0 | 0 | 0 | 0 | 46.5 | 45.8 | 46.9 | 64.5 |
| N120 | Man₀₋₄ | 57.4 | 46.2 | 34.8 | 20.1 | 15.5 | 9.8 | 14.2 | 6.6 | 15.0 | 7.8 |
| | Man₅ | 26.2 | 31.8 | 38.3 | 42.3 | 41.5 | 39.9 | 44.9 | 38.0 | 44.8 | 41.4 |
| | Man₆₋₉ | 4.3 | 7.3 | 14.3 | 23.1 | 25.3 | 29.9 | 25.6 | 40.0 | 14.1 | 32.4 |
| | Man₂₋₃F | 0.3 | 0 | 0 | 0 | 0 | 0 | 0 | 0 | 0 | 0 |
| | GlcNAc | 11.7 | 14.6 | 12.7 | 14.5 | 9.7 | 9.6 | 6.0 | 4.8 | 5.3 | 2.6 |
| | Gal | 0 | 0 | 0 | 0 | 7.8 | 10.8 | 2.8 | 4.0 | 0 | 0 |
| | Sia | 0 | 0 | 0 | 0 | 0 | 0 | 6.5 | 6.7 | 20.8 | 15.8 |

TABLE 6

| | Attached | | |
|---|---|---|---|
| | sugar chain | N48 | N120 |
| First molecular | Sia | 46.5 | 6.5 |
| species | Only Gal | 5.7 | 2.8 |
| | GlcNAc | 17.5 | 6.0 |
| Second molecular | Sia | 45.8 | 6.7 |
| species | Only Gal | 9.2 | 4.0 |
| | GlcNAc | 20.0 | 4.8 |

TABLE 7

| | | Order of | | |
|---|---|---|---|---|
| | | abundance | N48 | N120 |
| Sugar | First | 1 | SiaGalGlcNAcMan₃GlcNAc₂ | Man₅GlcNAc₂ |
| chain- | molecular | 2 | Man₃GlcNAc₂ | Man₇GlcNAc₂ |
| attaching | species | 3 | GlcNAcMan₃GlcNAc₂ | Man₂GlcNAc₂ |
| silkworm | | 4 | Man₅GlcNAc₂ | Man₈GlcNAc₂ |
| | Second | 1 | SiaGalGlcNAcMan₃GlcNAc₂ | Man₅GlcNAc₂ |
| | molecular | 2 | Man₅GlcNAc₂ | Man₇GlcNAc₂ |
| | species | 3 | SiaGalGlcNAcMan₅GlcNAc₂ | Man₈GlcNAc₂ |
| | | 4 | GlcNAcMan₃GlcNAc₂ | Man₆GlcNAc₂ |
| Gene | First | 1 | Man₃GlcNAc₂ | Man₃GlcNAc₂ |
| transgenic | molecular | 2 | GlcNAcMan₃GlcNAc₂ | Man₅GlcNAc₂ |
| silkworm | species | 3 | GlcNAc₂Man₃GlcNAc₂ | GlcNAcMan₃GlcNAc₂ |
| for control | | 4 | Man₂GlcNAc₂ | Man₂GlcNAc₂ |
| | Second | 1 | GlcNAcMan₃GlcNAc₂ | Man₅GlcNAc₂ |
| | molecular | 2 | Man₃GlcNAc₂ | Man₃GlcNAc₂ |
| | species | 3 | Man₂GlcNAc₂ | GlcNAcMan₃GlcNAc₂ |
| | | 4 | Man₃FucGlcNAc₂ | Man₄GlcNAc₂ |

As seen from these results, the sugar chain-attached asparagine residues N48 and N120 in hIFNγ also had a sialic acid-containing mammalian-type sugar chain attached to a non-reducing terminal in the transgenic silkworms comprising both of the GalT2 expression vector and the sialic acid-related gene expression vector. Thus, use of the glycosylation silkworm of the present invention enables a mammalian-type sugar chain to be attached to the protein of interest produced in a silk gland of the silkworm.

All publications, patents and patent applications cited herein are incorporated herein by reference in their entirety.

1. A mammalian-type glycosylation agent comprising one to three independent expression vector(s) comprising
a silk-spinning insect-derived middle and/or posterior silk gland promoter and
a gene encoding β1,4-galactosyltransferase or a nucleotide encoding an active fragment of the enzyme, functionally linked downstream of the promoter, and
genes encoding
UDP-acetylglucosamine 2-epimerase/N-acetylmannosamine kinase,
α2,6-sialyltransferase, and
Neu5Ac9-phosphate synthase and/or Neu5Ac9-phosphate phosphatase, or
nucleotides encoding active fragments of the enzymes, wherein
the genes encoding the enzymes or the nucleotides encoding active fragments of the enzymes are arranged so as to be under direct or indirect expression control of the middle and/or posterior silk gland promoter.

2. The mammalian-type glycosylation agent according to claim 1, wherein the β1,4-galactosyltransferase is GalT2.

3. The mammalian-type glycosylation agent according to claim 1, wherein the middle silk gland promoter is a promoter of sericin 1 gene, sericin 2 gene, or sericin 3 gene.

4. The mammalian-type glycosylation agent according to claim 1, wherein the posterior silk gland promoter is a promoter of fibroin H chain gene, fibroin L chain gene, or p25 gene.

5. The mammalian-type glycosylation agent according to claim 1, wherein the expression vector(s) further comprises a gene encoding CMP-Neu5Ac transporter or CMP-Neu5Ac synthase, or both, or a nucleotide encoding an active fragment of the enzyme.

6. The mammalian-type glycosylation agent according to claim 5, wherein the expression vector(s) comprises genes encoding β1,4-galactosyltransferase, UDP-acetylglucosamine 2-epimerase/N-acetylmannosamine kinase, α2,6-sialyltransferase, Neu5Ac9-phosphate synthase, Neu5Ac9-phosphate phosphatase, CMP-Neu5Ac transporter, and CMP-Neu5Ac synthase, or nucleotides encoding active fragments of the enzymes.

7. The mammalian-type glycosylation agent according to claim 1, wherein the expression vectors consist of
a first expression vector comprising the gene encoding β1,4-galactosyltransferase or the nucleotide encoding an active fragment of the enzyme, and
a second expression vector comprising the genes of UDP-acetylglucosamine 2-epimerase/N-acetylmannosamine kinase, α2,6-sialyltransferase, and Neu5Ac9-phosphate synthase and/or Neu5Ac9-phosphate phosphatase, or the nucleotides encoding active fragments of the enzymes.

8. The mammalian-type glycosylation agent according to claim 7, wherein
the gene encoding CMP-Neu5Ac transporter or CMP-Neu5Ac synthase, or both, or
the nucleotide encoding an active fragment of the enzyme is comprised in the second expression vector.

9. The mammalian-type glycosylation agent according to claim 1, wherein the genes encoding the enzymes or the nucleotides encoding active fragments of the enzymes are functionally linked downstream of the middle and/or posterior silk gland promoter.

10. The mammalian-type glycosylation agent according to claim 1, wherein the expression vector(s) is constituted by
(i) a first subunit comprising the middle and/or posterior silk gland promoter and a gene encoding a transcriptional control element, functionally linked downstream of the promoter, and
(ii) one or more second subunit(s) comprising a target promoter of the transcriptional control element and a gene(s) encoding one or more enzyme(s) selected from the group consisting of β1,4-galactosyltransferase, UDP-acetylglucosamine 2-epimerase/N-acetylmannosamine kinase, α2,6-sialyltransferase, Neu5Ac9-phosphate synthase, Neu5Ac9-phosphate phosphatase, CMP-Neu5Ac transporter, and CMP-Neu5Ac synthase, or a nucleotide(s) encoding an active fragment of the enzyme(s), functionally linked downstream of the promoter.

11. The mammalian-type glycosylation agent according to claim 10, wherein the transcriptional control element is yeast-derived GAL4 protein, and the target promoter thereof is UAS (upstream activating sequence).

12. The mammalian-type glycosylation agent according to claim 1, wherein the silk-spinning insect is a silkworm.

13. The mammalian-type glycosylation agent according to claim 1, wherein the mammalian type is a human type.

14. A transgenic silk-spinning insect capable of glycosylation with a mammalian-type sugar chain, comprising an expression vector(s) constituting a mammalian-type glycosylation agent according to claim 1.

15. A transgenic silk-spinning insect capable of glycosylation with a mammalian-type sugar chain, comprising an expression vector(s) constituting a mammalian-type glycosylation agent according to claim 10.

16. The transgenic silk-spinning insect capable of glycosylation with a mammalian-type sugar chain according to claim 15, wherein the first subunit and the second subunit reside on different chromosomes.

17. The transgenic silk-spinning insect capable of glycosylation with a mammalian-type sugar chain according to claim 14, wherein the silk-spinning insect is a silkworm.

18. The transgenic silk-spinning insect capable of glycosylation with a mammalian-type sugar chain according to claim 14, wherein the mammalian type is a human type.

19. A line producing a transgenic silk-spinning insect capable of glycosylation with a mammalian-type sugar chain, comprising only a second subunit(s) of an expression vector(s) constituting a mammalian-type glycosylation agent according to claim 10.

20. A method for preparing a transgenic silk-spinning insect capable of glycosylation with a mammalian-type sugar chain, comprising:
a mating step of mating a line producing a transgenic silk-spinning insect capable of glycosylation with a mammalian-type sugar chain according to claim 19, and a transgenic silk-spinning insect-producing line of the same species thereas having a first subunit(s) of an expression vector(s) constituting a mammalian-type glycosylation agent wherein the expression vector(s) is constituted by
(i) a first subunit comprising the middle and/or posterior silk gland promoter and a gene encoding a transcriptional control element, functionally linked downstream of the promoter, and
(ii) one or more second subunit(s) comprising a target promoter of the transcriptional control element and a gene(s) encoding one or more enzyme(s) selected from the group consisting of β1,4-galactosyltransferase, UDP-acetylglucosamine 2-epimerase/N-acetylmannosamine kinase, α2,6-sialyltransferase, Neu5Ac9-phosphate synthase, Neu5Ac9-phosphate phosphatase, CMP-Neu5Ac transporter, and CMP-Neu5Ac synthase, or a nucleotide(s) encoding an active fragment of the enzyme(s), functionally linked downstream of the promoter; and
a selection step of selecting a transgenic silk-spinning insect comprising the first subunit and the second subunit as the transgenic silk-spinning insect capable of glycosylation with a mammalian-type sugar chain from a first filial generation (F1).


SEQUENCE LISTING (99 sequences)

SEQ ID NO: 1 - DNA, 705 bp, Bombyx mori
  sericin 1 promoter
ctttgtatccctttttacgaaaattgcgaggacggaggagtatgaaatttcccacacttatagagaatacagagaagaagtgcacaatgctaatatttttttaaaataatgcataaaagatactttaaatcaataaagaaaacagcacacacactacataccatgtatttgacgcacacacgcatgtatactatttattgtcaaacttttgttcttgacgtctgtgttcaaactgagaatagattaaatattgtttgtctttattaatattttttaatagtgtagtcttggcgaaatttgtgattatagaagtataaaatacaatcataatagtgtacaaacttacaattccaattaattatagtcgaatttcgactactgcgggacctctagtattaataattctctttaaaaaaaaacagagcatcaaatactgcacaaatgtcaagcgggtctcaacgagccatgaataaattagaaatcaattaataacataaaataggcaaacaaaataaaaccatttacatagagaacgtttgttgaacaaaaacaataacttgtatacattgtttgcacaaatgtttgaaccgaaaatttattactctctacgtaagcttgatcaaacttcgttttcgtataaaacgcgttggcccaaccactttggcatagtcgtcttatcatcgggtctctaaggatcaagcgatc

SEQ ID NO: 2 - DNA, 2000 bp, Bombyx mori
  sericin 2 promoter
tggaaggatataataattatttttttttgctatgaaaaataataacttgtacttactccgataagtctttttattttttattgcttagatgggtggacgagctcacagactacctggtgctgagtggttaccggagcccatagacacaacgtaaatgcgccacccaccttgagatataagttctaaggtctaagtgtagttacaacggctgccccacctttcaaaccgaaacgcattactgcttaatggtagaaatgggcagtttgtaaagagatgatcaaagctgtaggctgatttttatctagtttcagtattattaagcattgagtaaattaataaagttacggactaagtttcatcgaagaacattctagtttttttcctacctattctgatagcctccggaggctatttcagcttctccttggtgtgtaggtgatctcacggggctcaaaccgggagtgttgctaacattggccctagcaagagcagtacttcgcagaatctaccacgatcggaaacgcgacccactgagaagatccggcgagaaactcagtgagctgtgtctatgggttaatttactcgtcgagccctgtttactgtttagggcgacgtcgactgttaccattcggtctacaggatcgagtgtgcattcttgtatcatcgttctattatcacgagtcattttgcgttttttcggatcccctggaagtcgtcgtggcctaagagataagaagtccggtgcattcgtgttgagcgatgcacctgtgttcgaatcctaggcgggtaccaatttttctaatgaattacgtacccaacaaatgttcacgattgccttccacggtgaaggaataacatcgtgcaataaaagtgaaacccgcaaaatccggtgcttttaagcttttcaagcaccggtcaccatcctcgttgaactcatcgatctacaagcgatctaatctatagacccaatccactaagatctcaccggatcttctcagtggttcgcattccagtggtagattcaattcgctgctcttgctagggctagtgttagcaaattccttcgggttaagcccgagagctcacctatccgtccgcgctaagctggaaaagccccttaagctgttttttttttgtatagcctttattgctaatactaaacaataactaataattttacatacagtaacaaattgttttaacttaaatctaatacatcggatttcccggttcagtgatcagcgtgtcctgtgacacataggcctcttccagctgctttcatttttctctattggtagcttttcttgaccagattgtctctccaatcatcttgatatcgtctgtccatcttctagcttgcctggctcttttcctttaaaccaggggtcgtgaatttcaatcctcacaggaagccgggattaggtgggagaatatagttccgatgttttgaatgctttatattttctgtggtcgaaaatgatactagagctacgcgtcgacaattgaatattatgctaactaccctctatttattaaaagacttttacgattcatttcgcacagaaccaatcgactgggtttagaggtttagcagtttgttgaatgaactcgttttcatcttcacgattagaggatcccaggtgttaggtaaaggatattctagattgcaggagatttttcataaataatcacgcgatggagcggtaatcagccaacatagtcgatcggcatcattattggagaccaaacaacacttcagttatccaagcgcgtcttaagtcgcattcggataatcttgaatagcctggaagtgaatttttaaaaagtttgtctcgaacaaacatcaattactttgtaattgaaccgaaaaaagaggataaacattattagcattcgttgtaatgaaatataatgttgacacagtttgaccgacgtgcactgtcttttgtggcaccggctatataaaggtggtctgtccgttctgagccacacgagtcatc

SEQ ID NO: 3 - DNA, 578 bp, Bombyx mori
  sericin 3 promoter
gcggagggactttagttctctctgcattctgtacggtatgtggagagtttctaccatcgtaccgcccgccaccagagtagagttcatccatactacctggagccactgcgttcatccacagtgcgtttccagagatcttttttgccacataccatccggctttggaatgagctcccctccacggtgcttcaaacgaggcttgtggagagtacttaacggtaggttggcttggctaggcttgactctgcccctggcattgctgaagttcatggacgacggtaaccacttaccatcaggtgggccgtatgctaatccgactacaagggcaacagcaaaaaaagttaattttacaaaagtatcataattccgaggctcgctaaagatgttcgtagcatatgttacagaaacaaaaaaaaaaacaatttaaatgcgttatagaaaaacaagtgtattaaacaaataattaattatttattttattggtaactgtttattcataaagggaatatttctccaacaaattagtataaatagccggcctttgggcgtttacagacagagcaatcgaagcttcgaag

SEQ ID NO: 4 - DNA, 870 bp, Bombyx mori
  Fib H promoter
acaaaactgccacacgcatttttttctccactgtaggttgtagttacgcgaaaacaaaatcgttctgtgaaaattcaaacaaaaatattttttcgtaaaaacacttatcaatgagtaaagtaacaattcatgaataatttcatgtaaaaaaaaaatactagaaaaggaatttttcattacgagatgcttaaaaatctgtttcaaggtagagatttttcgatatttcggaaaattttgtaaaactgtaaatccgtaaaattttgctaaacatatattgtgttgttttggtaagtattgacccaagctatcacctcctgcagtatgtcgtgctaattactggacacattgtataacagttccactgtattgacaataataaaacctcttcattgacttgagaatgtctggacagatttggctttgtatttttgatttacaaatgtttttttggtgatttacccatccaaggcattctccaggatggttgtggcatcacgccgattggcaaacaaaaactaaaatgaaactaaaaagaaacagtttccgctgtcccgttcctctagtgggagaaagcatgaagtaagttctttaaatattacaaaaaaattgaacgatattataaaattctttaaaatattaaaagtaagaacaataagatcaattaaatcataattaatcacattgttcatgatcacaatttaatttacttcatacgttgtattgttatgttaaataaaaagattaatttctatgtaattgtatctgtacaatacaatgtgtagatgtttattctatcgaaagtaaatacgtcaaaactcgaaaattttcagtataaaaaggttcaactttttcaaatcagcatcagttcgg

SEQ ID NO: 5 - DNA, 634 bp, Bombyx mori
  Fib L promoter
ggtacggttcgtaaagttcacctgcggctatattcagactcgccaagttacgtcagtcgtattgtaatgagcgatttagtgggcaacttcattctgttaattttgtgtcacggtgcgcgcgcatcgtaaaatttcactctcatagatttttcataacgtgcctaaagaagtataacttcaataatttaaattaaaaaaaaacatgcatagaataattatatgaattatttaaaatgtcatttaccgacattgacataacagacgacgttaacactacaaaacattttaattccacattgctacatattcaacagttaaatttgcgttaattctcgatgcgaacaaatataagaacaatcggatcaattagatcgctttgtttcgagcaacacttagtttaactagaggcgtacacctcaagaaatcatcttcattagaaactaaaccttaaaatcgcattaataaagcatagtcaattttaactgaaatgcaaaatcttttgaacgttagatgctgtcagcgttcgttggtacagttgtttgatatttattttaattgtctttttatatataaatagtggaacattaatcacggaatcctgtatagtatataccgattggtcacataacagacc

SEQ ID NO: 6 - DNA, 1344 bp, Bombyx mori
  p25 promptor
aagcttagataattcggcattgtgcgccactgagtcgcattatgctctgtaattggaaactaccaaacattgtgtaccctttaatgatattctaatctatatatataaaaatgaattgctgttcgttagtctcgctaaaactcgagaacggccggaccgatttggctaattttggtcttgaattatttgtggaagtccagagaagatttagaaggtttaaataaatatgaaaatgctcggaattaaataaaaataacaattttgttttttctttgatgtgttcccgtcggacggattcctttagtcttttatttatcgactagcgacccgccgcttcgcttcggaaacattaaaatacacatgataccaaaaaaattaaataatttttttttaaaaaaagtagcctatgttcatcaggtacaatgtcggcttctaatggaaaaagaatttttcaaatcggtccagtagtttcggagcctattcgaaacaaacaaacaaacaaatctttcctctttataatattagtatagatagtatagattgaggcactacgaagtctgccgggtcagctagtatactcataaataaggtcgacatctgttgatgatggtgatatcttcaaaattaccttagcgcaatgtagacttatacagtatttctgttttcctaagttaattaccgctgtagccaataccgtctttaccataagcgcacacggggcccggtccagggccgagtgtcgtcgagggggcccgaaagaccggcaagttctctcacacgtttattcccaaaacatttttgtcgggcacattacactttttccacaaatccgtaatcagaaggtatttagcaaggcatatactatgcctataatagaagattttgctcaacagaaatcccgagagaaaccgttatcgaaatcgtaaccaaaaaaccagcagcattctaatatcattaatgacatattatatcatactgtatttgattacctataataaagggtcatactcagtaaaaaaatgttaatataattcgctttttttactttccaaaagggcctcaaattcttgtgtgtccaagggccccatcttagtttaagacgtccctggctgtagcccagttactgccacacaaacatgcttaactcgcgccgcctacgtcgaggagaacattttgcgccttagaaaataaaatggcgtcgccgcggcgcaacaataagaacttaattcgtgcaattgtttccacgacgctatttatttaacgttattcgttgtgaggaacaatactttgtataattaatgttgatcagtgcctaacgacgcagttgtttattattcgcgcaac

SEQ ID NO: 7 - DNA, 1110 bp, Antheraea pernyi
  Fib H promoter
tccagcgttaccaatgagagcgcttcaaaattctttacaacttcattagaatacgtcgatttttctctacttcatataaatattctatagatgtgtttgctataacataaatacttttaaaaaaatgtctcaacggttgtgaaaactgtcaaaatctgttgcgtagttcagaaaaactaaggaaacatacagaaaatttattttacaaaagtacggagatatataaaaatatttcgattactttagaattacaataaaactatttgacaatttgattgcaaatatagaccatgacaacaccacatctttgttatctaaaacacgtagcgacaacactccttgaacgttgttcgaggattactacgataattggcggttttttttccgcaccgcaagaaaagagtagaaatgtaccgtatttaaatccagtgcggaaattttcacgcagaatgcgtttccatacaattctataggttacatatcttgcggaaataaattcgtgccaaaaagccgaagtgcggggactaataaagattttatttggcattccttctaacctttagatataaatttctgtacgcgcgtatgtcactgaactccccctaaacggctggactaattttgatgaaatattgtttgtgtgttctagtggatccgagaattgtttaaattcgcaaatccggtaggtgaacccgcggttgacttttagattttttttattatcaacaacaacgtccgcccggcccgctagttatgtatgtatttgtaaatgtaatctcaaaccgttcctgttggatcgacatttaatatgtttaagtgaattaattaacgtataacagtcataagaaaatattgcaataaaatcccatcatttattctttagagacaatataaccaaacaacaataagaatcagaatgtaattactctacattgttcatgataggggtttaactatgatattgttttaattctataggattcattactttatcattttgtcaatatttaaaattgtttatttgaaatagttaacgacattacaaagttttcgtataaaagggcgccaaagtctggtctcattatcagttcggttccagctctcataacc

SEQ ID NO: 8 - DNA, 1110 bp, Antheraea pernyi; identical to SEQ ID NO: 7
  Fib L promoter

SEQ ID NO: 9 - protein, 398 aa, Homo sapiens
  GalT1
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 10 - DNA, 1194 bp, Homo sapiens
  B4GALT1
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagc

SEQ ID NO: 11 - protein, 399 aa, Rattus norvegicus
  GALT1
MRFREPFLGGSAAMPGATLQRACRLLVAVCALHLGVTLVYYLSGRDLSRLPQLVGVSSSLQGGTNGAAASKQPSGELRPRGARPPPPLGVSPKPRPGSDSSPDAASGPGLKSNLTSVPMPTSTGLLTLPACPEESPLLVGPMVIDFNIPVDLELLAKKNPEIKMGGRYFPKDCISPHKVAIIIPFRNRQEHLKYWLYYLHPVLQRQQLDYGIYVINQAGDTMFNRAKLLNVGFQEALKDYDYNCFVFSDVDLIPMDDHNAYRCFSQPRHISVAMDKFGFSLPYVQYFGGVSALSKQQFLTINGFPNNYWGWGGEDDDIFNRLVHKGMSISRPNAVVGRCRMIRHSRDKKNEPNPQRFDRIAHTKETMRLDGLNSLTYQVLDIQRYPLYTKITVDIGTPR

SEQ ID NO: 12 - DNA, 1197 bp, Rattus norvegicus
  B4GALT1
atgaggtttcgtgagccgttcctgggcggcagcgccgcgatgccgggcgcgaccctgcagcgggcctgccgcctgctcgtggcggtctgcgcgctgcaccttggcgtcaccctggtctattacctctccggtcgcgatctgagccgcctgccccaactggtcggagtctcctcttcactgcaaggcggcacgaacggcgccgccgccagcaagcagccctcgggagagctccggccccggggcgcgcggccgccgcctcctttaggcgtctccccgaagcctcgcccgggttctgactccagccctgatgcggcttctggccccggcctgaagagcaacttgacttcggtgccaatgcccaccagcactggattgttgactctgcctgcttgccctgaggagtccccgctgctcgttggccccatggtgattgactttaatattcctgtggatctggagcttttggcaaagaagaacccagagataaagatgggcggccgttacttccccaaggactgtatctcccctcacaaggtggccatcattatcccattccgtaaccggcaggagcacctcaaatactggctgtattatttgcatccagtccttcagcgccagcaactcgactatggcatctacgtcatcaatcaggctggagacaccatgtttaatcgagctaagctgctcaacgttggctttcaagaggccttgaaagactatgactacaactgctttgtgttcagtgatgtggacctcattccaatggatgaccataatgcctacaggtgcttttcacagccacggcatatttctgtcgcaatggacaagttcgggtttagcctgccttacgttcagtattttggaggtgtctccgctctcagtaaacaacagttccttaccatcaatggatttcctaataattactggggctggggaggagaagatgatgacatttttaacagattagttcataaaggcatgtctatatcacgcccaaatgctgtggtaggcaggtgtcgcatgatccggcactcaagagacaagaaaaatgagcccaaccctcagaggtttgaccggatcgcacatacaaaggaaacgatgcgccttgatggtttgaactcacttacctaccaggtgttggacatacagagatacccgttatataccaaaatcacagtggacatcgggacaccaaga

SEQ ID NO: 13 - protein, 399 aa, Mus musculus
  GALT1
MRFREQFLGGSAAMPGATLQRACRLLVAVCALHLGVTLVYYLSGRDLSRLPQLVGVSSTLQGGTNGAAASKQPPGEQRPRGARPPPPLGVSPKPRPGLDSSPGAASGPGLKSNLSSLPVPTTTGLLSLPACPEESPLLVGPMLIDFNIAVDLELLAKKNPEIKTGGRYSPKDCVSPHKVAIIIPFRNRQEHLKYWLYYLHPILQRQQLDYGIYVINQAGDTMFNRAKLLNIGFQEALKDYDYNCFVFSDVDLIPMDDRNAYRCFSQPRHISVAMDKFGFSLPYVQYFGGVSALSKQQFLAINGFPNNYWGWGGEDDDIFNRLVHKGMSISRPNAVVGRCRMIRHSRDKKNEPNPQRFDRIAHTKETMRFDGLNSLTYKVLDVQRYPLYTQITVDIGTPR

SEQ ID NO: 14 - DNA, 1197 bp, Mus musculus
  B4GALT1
atgaggtttcgtgagcagttcctgggcggcagcgccgcgatgccgggcgcgaccctgcagcgggcctgccgcctgctcgtggccgtctgcgcgctgcacctcggcgtcaccctcgtctattacctctctggccgcgatctgagccgcctgccccagttggtcggagtctcctctacactgcagggcggcacgaacggcgccgcagccagcaagcagcccccaggagagcagcggccgcggggtgcgcggccgccgcctcctttaggcgtctccccgaagcctcgcccgggtctcgactccagccctggtgcagcttctggccccggcttgaagagcaacttgtcttcgttgccagtgcccaccaccactggactgttgtcgctgccagcttgccctgaggagtccccgctgctcgttggccccatgctgattgactttaatattgctgtggatctggagcttttggcaaagaagaacccagagataaagacgggcggccgttactcccccaaggactgtgtctctcctcacaaggtggccatcatcatcccattccgtaaccggcaggagcatctcaaatactggctgtattatttgcatcccatccttcagcgccagcaactcgactatggcatctacgtcatcaatcaggctggagacaccatgttcaatcgagctaagctgctcaatattggctttcaagaggccttgaaggactatgattacaactgctttgtgttcagtgatgtggacctcattccgatggacgaccgtaatgcctacaggtgtttttcgcagccacggcacatttctgttgcaatggacaagttcgggtttagcctgccatatgttcagtattttggaggtgtctctgctctcagtaaacaacagtttcttgccatcaatggattccctaataattattggggttggggaggagaagatgacgacatttttaacagattagttcataaaggcatgtctatatcacgtccaaatgctgtagtagggaggtgtcgaatgatccggcattcaagagacaagaaaaatgagcccaatcctcagaggtttgaccggatcgcacatacaaaggaaacgatgcgcttcgatggtttgaactcacttacctacaaggtgttggatgtacagagatacccgttatatacccaaatcacagtggacatcgggacaccgaga

SEQ ID NO: 15 - protein, 372 aa, Homo sapiens
  GalT2
MSRLLGGTLERVCKAVLLLCLLHFLVAVILYFDVYAQHLAFFSRFSARGPAHALHPAASSSSSSSNCSRPNATASSSGLPEVPSALPGPTAPTLPPCPDSPPGLVGRLLIEFTSPMPLERVQRENPGVLMGGRYTPPDCTPAQTVAVIIPFRHREHHLRYWLHYLHPILRRQRLRYGVYVINQHGEDTFNRAKLLNVGFLEALKEDAAYDCFIFSDVDLVPMDDRNLYRCGDQPRHFAIAMDKFGFRLPYAGYFGGVSGLSKAQFLRINGFPNEYWGWGGEDDDIFNRISLTGMKISRPDIRIGRYRMIKHDRDKHNEPNPQRFTKIQNTKLTMKRDGIGSVRYQVLEVSRQPLFTNITVDIGRPPSWPPRG

SEQ ID NO: 16 - DNA, 1116 bp, Homo sapiens
  B4GalT2
atgagcagactgctgggggggacgctggagcgcgtctgcaaggctgtgctccttctctgcctgctgcacttcctcgtggccgtcatcctctactttgacgtctacgcccagcacctggccttcttcagccgcttcagtgcccgaggccctgcccatgccctccacccagctgctagcagcagcagcagcagcagcaactgctcccggcccaacgccaccgcctctagctccgggctccctgaggtccccagtgccctgcccggtcccacggctcccacgctgccaccctgtcctgactcgccacctggtcttgtgggcagactgctgatcgagttcacctcacccatgcccctggagcgggtgcagagggagaacccaggcgtgctcatgggcggccgatacacaccgcccgactgcaccccagcccagacggtggcggtcatcatcccctttagacaccgggaacaccacctgcgctactggctccactatctacaccccatcttgaggcggcagcggctgcgctacggcgtctatgtcatcaaccagcatggtgaggacaccttcaaccgggccaagctgcttaacgtgggcttcctagaggcgctgaaggaggatgccgcctatgactgcttcatcttcagcgatgtggacctggtccccatggatgaccgcaacctataccgctgcggcgaccaaccccgccactttgccattgccatggacaagtttggcttccggcttccctatgctggctactttggaggtgtgtcaggcctgagtaaggctcagtttctgagaatcaatggcttccccaatgagtactggggctggggtggcgaggatgatgacatcttcaaccggatctccctgactgggatgaagatctcacgcccagacatccgaatcggccgctaccgcatgatcaagcacgaccgcgacaagcataacgaacctaaccctcagaggtttaccaagattcaaaacacgaagctgaccatgaagcgggacggcattgggtcagtgcggtaccaggtcttggaggtgtctcggcaaccactcttcaccaatatcacagtggacattgggcggcctccgtcgtggccccctcggggc

SEQ ID NO: 17 - protein, 369 aa, Rattus norvegicus
  GalT2
MSRLLGGTLERVCKAVLLLCLLHFLVAVILYFDVYAQHLAFFSRFSTRSPAHALYPAASSSTNCSRPNTTAASSGLPEVPSARPGPTAPVIPPCPDVPPGLVGRVVIEFTSPMPLERVQRENPGVLLGGRYSPPDCTPAQTVAVIIPFRHREHHLRYWLHYLHPMLRRQRLRYGVYVINQHGEETFNRAKLLNVGFLEALKEDATYDCFIFSDVDLVPMDDRNLYRCGDQPRHFAIAMDKFGFRLPYASYFGGVSGLSKAQFLRINGFPNEYWGWGGEDDDIFNRISLTGMKISRPDVRIGRYRMIKHDRDKHNEPNPQRFNKIQNTKMSMKWDGIGSVRYRVLEVSRQPLFTNITVDIGQPMSWLTQG

SEQ ID NO: 18 - DNA, 1107 bp, Rattus norvegicus
  B4GalT2
atgagcagactgctggggggtacgctggagcgggtctgtaaggctgtgctccttctctgcctgctgcacttccttgtggctgtcatcctctactttgacgtctacgcccagcacctggcctttttcagccgcttcagtacccgaagcccagcccatgccctctaccctgcagccagcagcagtaccaactgctctcggcccaacaccactgctgccagctccgggctccctgaagtgcccagtgccaggcctggccccacagctccagtcattccaccctgtcctgatgtgccgcctggtcttgtgggccgagtggtcatcgaattcacctcacccatgcctctggaacgggtgcagagggagaacccaggcgtgctcctgggcggccgctattctccacctgactgcaccccagcccaaacggtggcagtcatcatcccctttagacaccgggagcaccacctacgctattggctccactatctgcaccccatgttgaggcgacagcggctgcgctacggtgtctatgtcatcaaccagcatggcgaggagaccttcaaccgagccaagctgctcaacgtgggcttcctagaggcactgaaagaggatgccacctatgactgcttcatcttcagtgatgtggacctggtccctatggatgaccgcaatctgtaccgttgtggtgaccagccccgccacttcgccattgccatggacaagtttggcttccggttgccctatgccagctactttggaggtgtgtcaggcctgagcaaggcccagtttctgaggattaatggttttcccaacgagtactggggctggggcggcgaggacgatgacatcttcaaccggatctccctgactgggatgaagatctcacgcccagacgtccggataggccgctaccgcatgatcaagcacgaccgggacaaacataacgagcccaaccctcagaggtttaataagattcaaaacacaaagatgagcatgaagtgggacggcattggatccgtgcggtaccgcgtcttggaagtatctcggcaaccactcttcaccaacatcacagtggacattgggcagcccatgtcatggctcactcaaggc

SEQ ID NO: 19 - protein, 369 aa, Mus musculus
  GalT2
MSRLLGGTLERVCKAVLLLCLLHFLVAVILYFDVYAQHLAFFSRFSTRSPAHALYPAASSSTNCSRPNATAASSGLPEVPSARPGPTAPVIPPCPDVPPGLVGRVVIEFTSPMPLERVQRENPGVLLGGRYSPPDCTPAQTVAVIIPFRHREHHLRYWLHYLHPMLRRQRLRYGVYVINQHGEETFNRAKLLNVGFLEALKEDAAYDCFIFSDVDLVPMDDRNLYRCGDQPRHFAIAMDKFGFRLPYASYFGGVSGLSKAQFLRINGFPNEYWGWGGEDDDIFNRISLTGMKISRPDVRIGRYRMIKHDRDKHNEPNPQRFNKIQNTKMSMKWDGIGSVRYRVLEVSRQPLFTNITVDIGQPMSWLTQG

SEQ ID NO: 20 - DNA, 1107 bp, Mus musculus
  B4GalT2
atgagcagactgctgggggggacgctggagcgggtctgcaaggctgtgctccttctctgcctgctgcacttccttgtggccgtcatcctctactttgacgtctatgcccagcacctggcctttttcagccgcttcagtacccgaagcccagcccatgccctctaccctgcagccagcagcagtaccaactgctctcggcccaacgccactgccgctagctccgggctgcctgaagtgcccagtgccaggcctggtcccacagctccagtcattccgccctgtcctgacgtgccgcctggtcttgtgggccgagtggtcatcgaattcacctcacccatgcctctggaacgggtgcagagggaaaatccaggcgtgctcctgggcggccgctattctccacccgactgcaccccagcccaaacggtggcagtcatcatcccctttagacaccgggagcaccacctacgctattggctccactatctgcaccccatgttgaggcggcagcggctgcgctacggtgtctatgtcatcaaccagcatggcgaggagaccttcaaccgagccaagctgctcaacgtgggcttcctagaggcactgaaggaggatgctgcctatgactgcttcatcttcagtgatgtggacctggtccctatggatgaccgcaatctgtaccgttgtggtgaccagccccgccactttgccatcgccatggacaagtttggcttccggttgccctatgctagctactttggaggtgtgtcgggcctgagcaaggcccagtttctgaggatcaatggctttcccaacgagtactggggctggggtggcgaggatgatgacatcttcaaccggatctccctgactgggatgaagatctcacgcccggatgtccggattggccgctaccgcatgatcaagcacgaccgggacaaacataacgagcccaaccctcagaggtttaataagattcaaaacacaaagatgagcatgaagtgggacggcattggatccgtgcggtaccgagtcttggaagtgtctcggcaaccgctcttcaccaacatcacagtggacattggacagcccatgtcgtggctcactcaaggc

SEQ ID NO: 21 - protein, 393 aa, Homo sapiens
  GalT3
MLRRLLERPCTLALLVGSQLAVMMYLSLGGFRSLSALFGRDQGPTFDYSHPRDVYSNLSHLPGAPGGPPAPQGLPYCPERSPLLVGPVSVSFSPVPSLAEIVERNPRVEPGGRYRPAGCEPRSRTAIIVPHRAREHHLRLLLYHLHPFLQRQQLAYGIYVIHQAGNGTFNRAKLLNVGVREALRDEEWDCLFLHDVDLLPENDHNLYVCDPRGPRHVAVAMNKFGYSLPYPQYFGGVSALTPDQYLKMNGFPNEYWGWGGEDDDIATRVRLAGMKISRPPTSVGHYKMVKHRGDKGNEENPHRFDLLVRTQNSWTQDGMNSLTYQLLARELGPLYTNITADIGTDPRGPRAPSGPRYPPGSSQAFRQEMLQRRPPARPGPLSTANHTALRGSH

SEQ ID NO: 22 - DNA, 1179 bp, Homo sapiens
  B4GalT3
atgttgcggaggctgctggagcggccttgcacgctggccctgcttgtgggctcccagctggctgtcatgatgtacctgtcactggggggcttccgaagtctcagtgccctatttggccgagatcagggaccgacatttgactattctcaccctcgtgatgtctacagtaacctcagtcacctgcctggggccccagggggtcctccagctcctcaaggtctgccctactgtccagaacgatctcctctcttagtgggtcctgtgtcggtgtcctttagcccagtgccatcactggcagagattgtggagcggaatccccgggtagaaccagggggccggtaccgccctgcaggttgtgagccccgctcccgaacagccatcattgtgcctcatcgtgcccgggagcaccacctgcgcctgctgctctaccacctgcaccccttcttgcagcgccagcagcttgcttatggcatctatgtcatccaccaggctggaaatggaacatttaacagggcaaaactgttgaacgttggggtgcgagaggccctgcgtgatgaagagtgggactgcctgttcttgcacgatgtggacctcttgccagaaaatgaccacaatctgtatgtgtgtgacccccggggaccccgccatgttgccgttgctatgaacaagtttggatacagcctcccgtacccccagtacttcggaggagtctcagcacttactcctgaccagtacctgaagatgaatggcttccccaatgaatactggggctggggtggtgaggatgacgacattgctaccagggtgcgcctggctgggatgaagatctctcggccccccacatctgtaggacactataagatggtgaagcaccgaggagataagggcaatgaggaaaatccccacagatttgacctcctggtccgtacccagaattcctggacgcaagatgggatgaactcactgacataccagttgctggctcgagagctggggcctctttataccaacatcacagcagacattgggactgaccctcggggtcctcgggctccttctgggccacgttacccacctggttcctcccaagccttccgtcaagagatgctgcaacgccggcccccagccaggcctgggcctctatctactgccaaccacacagccctccgaggttcacac

SEQ ID NO: 23 - protein, 395 aa, Rattus norvegicus
  GalT3
MLRRLLERPCTLALLVGSQLAVMMYLSLGGFRSLSALFGRDPGPTFDYSHPHDVYSNLSHLPGAPGAAGAPLAQVLPDCPERSPFLVGPVSVSFSPVPSLAEIVERNPRVESGGRYRPAGCEPRSRTAIIVPHRAREHHLRLLLYHLHPFLQRQQLAYGIYVIHQAGNGTFNRAKLLNVGVREALRDEEWDCLFLHDVDLLPENDHNLYVCDPRGPRHVAVAMNKFGYSLPYPQYFGGVSALTPDQYLKMNGFPNEYWGWGGEDDDIATRVRLAGMKISRPPTSVGHYKMVKHRGDKGNEENPHRFDLLVRTQNSWTQDGMNSLTYRLLARELGPLYTNITADIGTDPRGPRAPSGPRYPPGSSQAFRQEMLQRRPPARPGPLPTANHTAPHGSH

SEQ ID NO: 24 - DNA, 1185 bp, Rattus norvegicus
  B4GalT3
atgttgcggaggctgctggagaggccctgcacactggccctgcttgtgggctcccaactggcagttatgatgtatctgtcactagggggcttccgaagtcttagtgccctgtttggtcgagatccaggcccaacattcgactattctcatccccatgatgtctatagtaacctcagtcacctgcctggagcccctggagctgcaggggctcctctagctcaagttctgcctgactgtccagaaagatcgcctttcttagtgggtcctgtgtcagtatcctttagcccagtgccatcactagcagagatcgtggagcggaatccccgggtggaatcagggggccggtaccgtcctgcaggatgtgagcctcgctcccgaacagccataattgtgccccaccgtgcccgggagcaccacctccgactgctactctatcacctgcaccccttcctgcagcgccagcagcttgcttatggcatctatgtcatccaccaggctggaaatggaacgtttaacagggccaaactgctgaacgtaggggtcagggaagcccttcgtgacgaagagtgggactgcttgtttttacacgatgtggacctccttccagaaaacgaccataacctgtatgtgtgtgacccccggggaccccgccatgttgctgtcgccatgaacaagtttggatacagccttccgtacccccagtactttggcggagtttcagcactcactcctgaccagtacctgaagatgaatggcttccccaatgaatactggggctggggtggcgaggatgacgacattgctaccagggttcgcctggctgggatgaagatttcccgaccacccacctctgtgggacactacaagatggtgaagcacagaggggataaaggaaatgaggaaaacccccacagatttgacctcctggtccgtacccagaattcttggacacaagatggaatgaactcactaacataccgactgctggcaagagagctgggtcctctctacaccaacatcactgcagacattgggactgaccctcggggtccccgggctccctctggtccccgatacccaccaggttcctcccaggccttccgtcaggagatgctgcaacgccggcccccagctaggcctggccctctgcctactgccaaccacacagctccccatggttcacac

SEQ ID NO: 25 - protein, 395 aa, Mus musculus
  GalT3
MLRRLLERPCTLALLVGSQLAVMMYLSLGGFRSLSALFGRDPGPTFDYSHPHDVYSNLSHLPAAPGAAGAPPAQALPYCPERSPFLVGPVSVSFSPVPSLAEIVERNPRVESGGRYRPAGCEPRSRTAIIVPHRAREHHLRLLLYHLHPFLQRQQLAYGIYVIHQAGNGTFNRAKLLNVGVREALRDEEWDCLFLHDVDLLPENDHNLYVCDPRGPRHVAVAMNKFGYSLPYPQYFGGVSALTPDQYLKMNGFPNEYWGWGGEDDDIATRVRLAGMKISRPPTSVGHYKMVKHRGDKGNEENPHRFDLLVRTQNSWTQDGMNSLTYRLLARELGPLYTNITADIGTDPRGPRAPSGPRYPPGSSQAFRQEMLQRRPPARPGPLPTANHTAPRGSH

SEQ ID NO: 26 - DNA, 1185 bp, Mus musculus
  B4GalT3
atgttgcggaggctgctggagagaccctgtacattggccctgcttgtgggctcccaactggcagtgatgatgtatctgtcactagggggcttccgaagtcttagtgccctgtttggtcgagatccgggcccaacattcgactattctcatccccacgatgtctatagtaacctcagtcacctgcccgcagcccctggagctgcaggggctcctccggctcaagcattgccctactgtccagaaagatcacccttcttagtgggtcctgtgtcagtatcctttagcccggtgccgtcactagcagagattgtggagcggaatccccgggtggaatcagggggccggtaccgtcctgcagggtgtgagcctcgctcccgaacagccataattgtgccccatcgtgcccgggagcaccatcttcggctgctgctctatcacctgcaccctttcctgcagcgccagcagcttgcgtacggcatttatgtcatccaccaggctggaaatggaacgtttaacagggcaaagctgctgaacgtaggggtgagggaagcccttcgtgatgaagaatgggactgcttgttcttacacgacgtggacctccttccagaaaacgaccataacctgtatgtgtgcgacccccggggaccccgccacgttgctgttgccatgaacaagtttggatacagcctcccgtacccccagtactttggcggagtttcagcgctcactcccgaccagtacctgaagatgaacggcttccccaacgagtactggggctggggtggcgaggatgacgacattgctaccagggtccgcctggctgggatgaagatctctcgaccacctacctctgtgggacactataagatggtgaagcacagaggggataaaggaaatgaggaaaatccccacagatttgacctcctggtccgtacccagaattcttggacacaagatggaatgaactcactaacgtaccgactgctggcaagagagctgggtcctctctataccaacatcactgcagacatcgggactgaccctcggggtccccgggctccctctggtccccgatacccaccaggttcctcccaggccttccgtcaagagatgctgcaacgccggcccccagctaggcctggccctctgcccactgccaaccacacagctccccgtggctcacac

SEQ ID NO: 27 - protein, 722 aa, Homo sapiens
  GNE
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 28 - DNA, 2166 bp, Homo sapiens
  GNE
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctac

SEQ ID NO: 29 - protein, 722 aa, Rattus norvegicus
  GNE
MEKNGNNRKLRVCVATCNRADYSKLAPIMFGIKTEPAFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKCKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERISLILQMCVEAAAEAVKLNCRILGVGISTGGRVNPQEGVVLHSTKLIQEWNSVDLRTPLSDTLHLPVWVDNDGNCAAMAERKFGQGKGQENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNVKAQSILRTAGTALGLGVVNILHTMNPSLVILSGVLASHYIHIVRDVIRQQALSSVQDVDVVVSDLVDPALLGAASMVLDYTTRRIH

SEQ ID NO: 30 - DNA, 2166 bp, Rattus norvegicus
  GNE
atggagaagaacgggaataaccggaagcttcgggtttgcgttgccacctgcaaccgagccgattactccaaactggcccccatcatgttcggcattaagacggagcctgcgttcttcgagctcgacgtggtggtgctgggctctcacctgatcgacgactacggaaacacataccgcatgattgagcaggacgactttgacatcaacaccaggctacacacgattgttagaggggaagacgaagcagccatggtagagtcagtgggcctagcgctagtgaagctaccggatgtcctcaaccgcctgaagcctgacatcatgattgttcacggagaccgatttgacgccctcgctctggctacatctgctgccctgatgaacatccgcatccttcacattgaaggaggagaggtcagcgggactattgatgactctatcagacacgccataacaaaactggctcactaccacgtgtgctgcaccaggagtgcagagcaacacctgatctccatgtgtgaggaccacgaccgcatccttttggctggctgcccttcctatgacaaactgctctcagccaagaataaagactatatgagcatcattcggatgtggctaggtgatgatgtaaaatgtaaagattacattgttgccctgcaacacccggtgaccaccgacattaagcattccataaagatgttcgaactgacactggatgctcttatctcatttaacaagaggaccctagttctgtttccaaatatcgatgcaggcagcaaggagatggttcgagtgatgcggaagaagggcatcgagcatcaccccaatttccgcgcagtcaagcacgtcccgtttgaccagttcattcagctggtcgcccacgctggctgcatgattgggaatagcagctgtggagtgcgtgaggttggcgcctttggaacccctgtgatcaacctgggcacgcggcagataggaagagaaacgggggagaatgttcttcatgtccgggatgctgacacccaagacaaaatattacaagcactacacctccagttcggtaaacagtacccttgctcaaagatatatggggatggaaatgctgttccaaggattttaaagtttctcaaatccatcgaccttcaagagccactacagaagaaattctgcttccctcccgtgaaggagaacatctctcaggatattgaccatatcctcgaaactctgagtgccttggctgttgatctcggggggacgaatctgagagtggcgatagttagcatgaagggtgaaatagttaagaagtacacccagttcaatcctaaaacctatgaggaaaggattagtctaatcctgcagatgtgtgtggaagcggcagcagaagccgtgaagctcaattgcagaattctgggagtaggcatctccacaggtggccgtgtgaatccccaggaaggagttgtgctgcactcgaccaagctgatacaggagtggaactctgtggacctcaggacaccactctccgacaccctgcatctccccgtgtgggtggacaacgacggcaactgcgctgccatggcggagaggaagtttggccaaggaaaaggacaggagaactttgtgacgctcatcacagggacagggatcggtgggggaatcatccaccagcacgagctgatccacggcagctccttctgtgcggcagagcttggccacctcgtggtgtctctggatggtcctgactgctcctgtggaagccatgggtgcattgaagcctacgcctctggaatggccttgcagagggaagcaaagaagctccacgacgaggacctgctcttggtggaagggatgtcagtgccaaaagacgaagctgtgggcgccctccatctcatccaagccgccaagctgggcaacgtgaaggcccagagcatcttacggacagctggaactgctttgggactcggagttgtgaatatcctccacactatgaatccttccctggtgatcctgtctggagtcctggctagtcactacatccacattgtgagggacgtcatccgccagcaagccctgtcctccgtgcaggatgtggatgtagtggtttcagacttggttgacccggccctgcttggtgcggccagcatggttctggactacacgacccgcaggatccac

SEQ ID NO: 31 - protein, 722 aa, Mus musculus
  GNE
MEKNGNNRKLRVCVATCNRADYSKLAPIMFGIKTEPAFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKCKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERISLILQMCVEAAAEAVKLNCRILGVGISTGGRVNPQEGVVLHSTKLIQEWNSVDLRTPLSDTLHLPVWVDNDGNCAAMAERKFGQGKGQENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNVKAQSILRTAGTALGLGVVNILHTMNPSLVILSGVLASHYIHIVKDVIRQQALSSVQDVDVVVSDLVDPALLGAASMVLDYTTRRIH

SEQ ID NO: 32 - DNA, 2166 bp, Mus musculus
  GNE
atggagaagaacgggaacaaccgaaagctccgggtttgcgttgccacctgcaaccgagctgactactccaaactggccccgatcatgttcggcatcaagacagagcccgcgttctttgagttggacgtggtggtgctcggctcccacctgattgacgactatggaaacacataccgcatgattgagcaagatgactttgacattaacaccaggctccacacgattgttagaggggaagatgaagcggccatggtagagtcggtaggcctagcgctcgtgaagctaccggacgtcctcaatcgcctgaagcccgacatcatgattgttcacggagaccgatttgacgcccttgctctggctacgtctgctgccttgatgaacatccgcatccttcacattgaaggaggcgaggtcagcgggaccattgatgactctatcagacacgccataacaaaactggctcactaccatgtgtgctgcactagaagtgcagagcagcacctgatctctatgtgcgaggaccacgaccgcatcctgttggcaggctgcccttcctatgacaaactgctctccgccaagaacaaagactatatgagcatcattcggatgtggctaggcgatgatgtaaaatgtaaggattacatcgttgccctgcagcatcccgtgaccactgacattaagcattccataaagatgtttgagctaacactggatgccctgatctcgtttaacaagaggaccctagttctgtttccaaatatcgatgcaggcagcaaggagatggttcgagtgatgcggaagaagggcatcgagcatcaccccaatttccgtgcagtcaagcacgtcccgtttgaccagttcatacagctggtcgcccacgctggctgcatgattgggaatagcagctgcggcgtgcgagaggttggcgctttcggaacacccgtgatcaacctgggcacaaggcagataggaagagaaaccggggagaatgttcttcatgtcagggatgctgacacccaagataaaatattgcaagcactacacctccagttcggcaaacagtacccttgctcaaagatatatggggatgggaatgctgttccaaggattttaaagtttctcaaatccattgaccttcaagagccactacagaagaaattctgcttcccccctgtaaaggagaacatctctcaagacattgaccacatcctggaaactctgagtgccttggctgttgatcttggcgggacaaacctgagggtggcaatagttagcatgaagggtgaaatcgttaagaagtacactcagttcaaccctaaaacctatgaagaaaggattagtttaatcctgcagatgtgtgtggaagctgccgcggaagctgtgaaactcaattgcagaattctgggagtaggcatctccacaggtggccgcgtgaatccccaggaaggagttgtgctgcattcaaccaagctgatccaggaatggaactccgtggacctcaggacacccctctccgacaccctgcatctccccgtgtgggtggacaatgacggcaactgtgccgccatggcagagaggaagttcggccaaggaaaaggacaggagaacttcgtgacgctcatcacggggacagggatcggtggggggatcatccaccagcacgaactgatccacggcagctccttctgcgcggcggagctcggccatctcgtggtgtccctggacggtcctgactgctcctgtggaagccatgggtgcatcgaagcgtacgcctctggaatggccttgcagagggaagcaaagaaactccatgatgaggacctgctcttggtggaagggatgtcagtaccaaaagacgaagctgtgggtgccctccatctcatccaggctgccaagctgggcaacgtgaaggcccagagcatcttacgaacagctggaactgctttgggacttggggttgtgaacatcctccacactatgaatccttccctggtgatcctgtctggagtcctggccagtcactacatccacatcgtgaaggacgtcatccgccagcaagccttgtcctccgtgcaggatgtggacgtggtggtctcagacttggtggacccggccctgcttggcgcagccagcatggttctggactacacaacgcgcaggatccac

SEQ ID NO: 33 - protein, 359 aa, Homo sapiens
  NANS
MPLELELCPGRWVGGQHPCFIIAEIGQNHQGDLDVAKRMIRMAKECGADCAKFQKSELEFKFNRKALERPYTSKHSWGKTYGEHKRHLEFSHDQYRELQRYAEEVGIFFTASGMDEMAVEFLHELNVPFFKVGSGDTNNFPYLEKTAKKGRPMVISSGMQSMDTMKQVYQIVKPLNPNFCFLQCTSAYPLQPEDVNLRVISEYQKLFPDIPIGYSGHETGIAISVAAVALGAKVLERHITLDKTWKGSDHSASLEPGELAELVRSVRLVERALGSPTKQLLPCEMACNEKLGKSVVAKVKIPEGTILTMDMLTVKVGEPKGYPPEDIFNLVGKKVLVTVEEDDTIMEELVDNHGKKIKS

SEQ ID NO: 34 - DNA, 1077 bp, Homo sapiens
  NANS
atgccgctggagctggagctgtgtcccgggcgctgggtgggcgggcaacacccgtgcttcatcattgccgagatcggccagaaccaccagggcgacctggatgtagccaagcgcatgatccgcatggccaaggagtgtggggctgattgtgccaagttccagaagagtgagctagaattcaagtttaatcggaaagccttggagaggccatacacctcgaagcattcctgggggaagacgtacggggagcacaaacgacatctggagttcagccatgaccagtacagggagctgcagaggtacgccgaggaggttgggatcttcttcactgcctctggcatggatgagatggcagttgaattcctgcatgaactgaatgttccatttttcaaagttggatctggagacactaataattttccttatctggaaaagacagccaaaaaaggtcgcccaatggtgatctccagtgggatgcagtcaatggacaccatgaagcaagtttatcagatcgtgaagcccctcaaccccaacttctgcttcttgcagtgtaccagcgcatacccgctccagcctgaggacgtcaacctgcgggtcatctcggaatatcagaagctctttcctgacattcccatagggtattctgggcatgaaacaggcatagcgatatctgtggccgcagtggctctgggggccaaggtgttggaacgtcacataactttggacaagacctggaaggggagtgaccactcggcctcgctggagcctggagaactggccgagctggtgcggtcagtgcgtcttgtggagcgtgccctgggctccccaaccaagcagctgctgccctgtgagatggcctgcaatgagaagctgggcaagtctgtggtggccaaagtgaaaattccggaaggcaccattctaacaatggacatgctcaccgtgaaggtgggtgagcccaaaggctatcctcctgaagacatctttaatctagtgggcaagaaggtcctggtcactgttgaagaggatgacaccatcatggaagaattggtagataatcatggcaaaaaaatcaagtct

SEQ ID NO: 35 - protein, 359 aa, Rattus norvegicus
  NANS
MPLELELCPGRWVGGQHPCFIIAEIGQNHQGDLDVAKRMIRTAKECGADCAKFQKSELEFKFNRKALERPYTSKHSWGKTYGEHKRHLEFSHDQYKELQSYAQEIGIFFTASGMDEMAVEFLHELNVPFFKVGSGDTNNFPYLEKTAKKGRPMVISSGMQSMDTMKQVYQIVKPLNPNFCFLQCTSAYPLQPEDANLRVISEYQKLFPDIPIGYSGHETGIAISVAAVALGAKVLERHITLDKTWKGSDHLASLEPGELAELVRSVRLVERALGSPAKQLLPCEMACNEKLGKSVVAKVKIPAGTILTLDMLTVKVGEPKGYPPEDIFNLVGKKVLVTIEEDDTVMEESVESQSKKIKA

SEQ ID NO: 36 - DNA, 1077 bp, Rattus norvegicus
  NANS
atgccgctggagctggagctgtgtcccgggcgctgggtgggtggacagcacccgtgcttcatcatcgcggagatcggccagaaccaccaaggagacttagatgtggccaagcgcatgatccgcactgccaaggagtgtggggccgactgcgctaagtttcagaagagtgagttggagttcaagtttaaccggaaggccctggagagaccatatacttcgaagcattcatgggggaagacttacggggagcacaaacgacatctagaattcagccacgatcagtacaaggagctgcagagctacgcgcaggagattggcatcttcttcactgcgtctggcatggatgagatggcagttgagtttctgcatgaactgaatgttccctttttcaaagttggatctggagacactaacaattttccctacctggaaaagacagccaagaaaggtcgtcctatggtgatctccagtgggatgcagtccatggacaccatgaagcaagtctatcagatcgtgaagcccctgaatcccaacttctgcttcctccagtgcaccagtgcgtacccactacagcctgaggatgccaacctgcgcgtcatctcggaataccagaagctctttcctgacattcccatagggtattctgggcacgagacaggcatcgccatatctgtagctgcagtggccctgggggccaaggtgttggaacgtcatataactttggacaagacctggaaggggagtgaccacttagcctcactggagcctggagaactggcagagctggtgcggtctgtgcgtctggtggagcgggcactgggctccccagccaagcagctcctgccctgtgagatggcctgcaacgagaagctcggcaagtccgtggtagccaaagtgaaaatcccagcaggcaccatcctgaccctggacatgctcactgtgaaggtgggggaacccaaaggctatcctcctgaagacatcttcaacctggtgggcaaaaaggtgctggtcactattgaagaagatgacacggtcatggaggaatccgtggaaagtcaaagcaagaaaatcaaggct

SEQ ID NO: 37 - protein, 359 aa, Mus musculus
  NANS
MPLELELCPGRWVGGKHPCFIIAEIGQNHQGDIDVAKRMIRTAKECGADCAKFQKSELEFKFNRKALERPYTSKHSWGKTYGEHKRHLEFSHDQYKELQSYAQEIGIFFTASGMDEMAVEFLHELNVPFFKVGSGDTNNFPYLEKTAKKGRPMVISSGMQSMDTMKQVYQIVKPLNPNFCFLQCTSAYPLQPEDANLRVISEYQKLFPDIPIGYSGHETGIAISVAAVALGAKVLERHITLDKTWKGSDHSASLEPGELAELVRSVRLVERALGSPTKQLLPCEMACNEKLGKSVVAKVKIPAGTTLTLDMLTVKVGEPKGYPPEDIFNLAGKKVLVTIEEDDTVMEESVESHSKKIKA

SEQ ID NO: 38 - DNA, 1077 bp, Mus musculus
  NANS
atgccgctggaactggagctgtgtcccgggcgctgggtgggtggaaagcacccgtgcttcatcatcgcggagatcggccagaaccaccaaggagacatagatgtggccaagcgcatgatccgcactgccaaggagtgtggggccgactgcgctaagtttcagaagagcgagttggagttcaagtttaaccggaaggccctggagagaccatatacttcgaagcattcatgggggaagacgtatggggagcacaagcggcatctggaattcagccacgaccagtacaaggagctgcagagctatgcgcaggagatcggcatcttcttcactgcctctggcatggacgagatggcagttgagtttctgcacgaactgaatgttccctttttcaaagttggatctggggacactaacaattttccctacctggaaaagacagccaagaaaggtcgtcctatggtgatctccagcgggatgcagtcaatggacaccatgaagcaagtctatcagatcgtgaagccgctgaatcccaacttctgcttcctccaatgcaccagcgcgtacccactacagcccgaggatgccaacctgcgcgtcatctcggaataccagaagctctttcccgacattcccatcgggtattccgggcacgagacgggcatcgccatatctgtggccgccgtggctctgggggccaaggtgttggaacgtcacataacgttggacaagacctggaaggggagtgaccactcagcctcgctggagcctggggagctggcagagctggtgcggtctgtgcgcctggtggagcgggccctgggctccccaaccaagcagctgctgccctgtgagatggcctgcaatgagaagctcggcaagtctgtggtagccaaagtgaaaatcccagcaggcaccaccctgaccctggacatgctcactgtgaaggtgggggagcccaaaggctatcctcctgaagacatcttcaacctagcgggcaaaaaggtgctggtcactatcgaagaagatgacacggtcatggaagaatccgtggaaagtcacagcaagaaaatcaaggct

SEQ ID NO: 39 - protein, 248 aa, Homo sapiens
  NANP
MGLSRVRAVFFDLDNTLIDTAGASRRGMLEVIKLLQSKYHYKEEAEIICDKVQVKLSKECFHPYNTCITDLRTSHWEEAIQETKGGAANRKLAEECYFLWKSTRLQHMTLAEDVKAMLTELRKEVRLLLLTNGDRQTQREKIEACACQSYFDAVVVGGEQREEKPAPSIFYYCCNLLGVQPGDCVMVGDTLETDIQGGLNAGLKATVWINKNGIVPLKSSPVPHYMVSSVLELPALLQSIDCKVSMST

SEQ ID NO: 40 - DNA, 744 bp, Homo sapiens
  NANP
atggggctgagccgcgtgcgggcggttttctttgacttggacaacactctcatcgacacggccggggcgagcaggagaggcatgttggaggtgataaaactcttacaatcaaaataccattataaagaagaggctgaaatcatctgtgataaagttcaagttaaactcagcaaggaatgttttcatccttacaatacatgcattactgatttaaggacttcacattgggaagaagcaatccaggaaacaaaaggtggtgcagccaatagaaaattggctgaagaatgttatttcctttggaaatctacacgtttacagcatatgacactagcagaagacgtcaaagccatgcttactgaacttcgaaaggaggtccgcctacttctattaacgaatggggacagacagacccagagggagaagattgaggcttgtgcctgtcagtcctattttgacgctgttgttgtaggtggagagcagagagaggagaaaccagcaccgtccatattttattactgctgcaatcttctcggagtacaacctggggactgtgtgatggtcggtgacacattagaaaccgacatccaaggaggcctcaatgcaggattgaaagcaacagtctggatcaataaaaatggaatagtgccactgaagtcctccccagttccgcattacatggtttcttctgtgctagagttacctgctctcttacaaagtatagactgcaaagtcagtatgtccact

SEQ ID NO: 41 - protein, 248 aa, Rattus norvegicus
  NANP
MGLSRVRAVFFDLDNTLIDTAGASRRGMLEVIKLLQSKYHYKEEAEVICDKVQVKLSKECFHPYSTCITDVRTSHWEEAIQETKGGADNRKLAEECYFLWKSTRLQHMTLEEDVKAMLTELRKEVRLLLLTNGDRQTQREKIEACACQSYFDAIVVGGEQKEEKPAPSIFYHCCDLLGVQPGDCVMVGDTLETDIQGGLNAGLKATVWINKSGGVPLTSSPMPHYMVSSVLELPALLQSIDCKVSMSV

SEQ ID NO: 42 - DNA, 744 bp, Rattus norvegicus
  NANP
atggggctgagtcgggtccgcgcggtcttctttgacctggacaacacactcatcgacacggccggggcgagcaggagaggcatgttggaggtaataaagctcttacaatcaaaataccactacaaagaagaggctgaagtcatctgcgataaagttcaagttaaactgagcaaggagtgctttcatccctatagtacatgcattacagatgtgaggacttcgcactgggaagaagcaatccaggaaaccaaaggtggtgccgacaataggaaattggctgaggaatgttatttcctgtggaaatctacacgcttacagcacatgaccctagaagaagatgtcaaagccatgctcactgaacttcgtaaagaggtccgcctactcttgttaacaaacggtgacagacagacacagagggaaaagatcgaggcttgtgcctgccagtcttactttgatgccattgttgtaggaggagaacagaaggaagagaaaccagcaccttccatattttatcactgctgtgatcttcttggagtgcagccaggggactgtgtgatggttggtgacacactagaaaccgatatacaaggaggcctgaatgcaggactgaaagccacggtctggataaacaagagtggaggagtgccactgacatcgtcccccatgcctcactatatggtttcctctgtgttagaattacctgctctcttgcaaagcatagattgcaaagtcagcatgtctgtg

SEQ ID NO: 43 - protein, 248 aa, Mus musculus
  NANP
MGLSRVRAVFFDLDNTLIDTAGASRRGMLEVIKLLQSKYHYKEEAEIICDKVQVKLSKECFHPYSTCITDVRTSHWEEAIQETKGGADNRKLAEECYFLWKSTRLQHMILADDVKAMLTELRKEVRLLLLTNGDRQTQREKIEACACQSYFDAIVIGGEQKEEKPAPSIFYHCCDLLGVQPGDCVMVGDTLETDIQGGLNAGLKATVWINKSGRVPLTSSPMPHYMVSSVLELPALLQSIDCKVSMSV

SEQ ID NO: 44 - DNA, 744 bp, Mus musculus
  NANP
atggggctgagtcgggtccgcgcggtcttctttgacctggacaacacactcatcgacacggccggggcgagcaggagaggcatgttggaggtaataaagctcttacagtcaaaataccactacaaagaagaggctgaaatcatctgtgataaagttcaagttaaactgagcaaggagtgctttcatccctatagtacatgcattacagatgtgaggacttcacactgggaagaagcaatccaggaaaccaaaggaggtgctgacaataggaaattggcggaggaatgttatttcctgtggaaatctacacgcttacagcacatgatcctagcagacgatgtcaaagccatgctcactgaacttcgaaaagaggtccgcctgctcctgttaacaaatggtgacagacagactcagagggaaaagatcgaggcctgcgcctgccagtcttactttgatgccattgttattggcggagaacagaaggaagagaaaccagcaccttccatattttatcactgctgtgatcttcttggagtgcagccaggtgactgtgtgatggttggtgacacactggaaaccgatatacaaggaggcctcaatgcaggactgaaagctacggtctggataaacaagagtggaagagtgccgctgacatcatcacccatgcctcactatatggtttcttctgtgctagaattacctgctctcttgcaaagcatagattgcaaagtcagcatgtctgtg

SEQ ID NO: 45 - protein, 434 aa, Homo sapiens
  CAMS
MDSVEKGAATSVSNPRGRPSRGRPPKLQRNSRGGQGRGVEKPPHLAALILARGGSKGIPLKNIKHLAGVPLIGWVLRAALDSGAFQSVWVSTDHDEIENVAKQFGAQVHRRSSEVSKDSSTSLDAIIEFLNYHNEVDIVGNIQATSPCLHPTDLQKVAEMIREEGYDSVFSVVRRHQFRWSEIQKGVREVTEPLNLNPAKRPRRQDWDGELYENGSFYFAKRHLIEMGYLQGGKMAYYEMRAEHSVDIDVDIDWPIAEQRVLRYGYFGKEKLKEIKLLVCNIDGCLTNGHIYVSGDQKEIISYDVKDAIGISLLKKSGIEVRLISERACSKQTLSSLKLDCKMEVSVSDKLAVVDEWRKEMGLCWKEVAYLGNEVSDEECLKRVGLSGAPADACSTAQKAVGYICKCNGGRGAIREFAEHICLLMEKVNNSCQK

SEQ ID NO: 46 - DNA, 1302 bp, Homo sapiens
  CAMS
atggactcggtggagaagggggccgccacctccgtctccaacccgcgggggcgaccgtcccggggccggccgccgaagctgcagcgcaactctcgcggcggccagggccgaggtgtggagaagcccccgcacctggcagccctaattctggcccggggaggcagcaaaggcatccccctgaagaacattaagcacctggcgggggtcccgctcattggctgggtcctgcgtgcggccctggattcaggggccttccagagtgtatgggtttcgacagaccatgatgaaattgagaatgtggccaaacaatttggtgcacaagttcatcgaagaagttctgaagtttcaaaagacagctctacctcactagatgccatcatagaatttcttaattatcataatgaggttgacattgtaggaaatattcaagctacttctccatgtttacatcctactgatcttcaaaaagttgcagaaatgattcgagaagaaggatatgattctgttttctctgttgtgagacgccatcagtttcgatggagtgaaattcagaaaggagttcgtgaagtgaccgaacctctgaatttaaatccagctaaacggcctcgtcgacaagactgggatggagaattatatgaaaatggctcattttattttgctaaaagacatttgatagagatgggttacttgcagggtggaaaaatggcatactatgaaatgcgagctgaacatagtgtggatatagatgtggatattgattggcctattgcagagcaaagagtattaagatatggctattttggcaaagagaagcttaaggaaataaaacttttggtttgcaatattgatggatgtctcaccaatggccacatttatgtatcaggagaccaaaaagaaataatatcttatgatgtaaaagatgctattgggataagtttattaaagaaaagtggtattgaggtgaggctaatctcagaaagggcctgttcaaagcagacgctgtcttctttaaaactggattgcaaaatggaagtcagtgtatcagacaagctagcagttgtagatgaatggagaaaagaaatgggcctgtgctggaaagaagtggcatatcttggaaatgaagtgtctgatgaagagtgcttgaagagagtgggcctaagtggcgctcctgctgatgcctgttctactgcccagaaggctgttggatacatttgcaaatgtaatggtggccgtggtgccatccgagaatttgcagagcacatttgcctactaatggaaaaggttaataattcatgccaaaaa

SEQ ID NO: 47 - protein, 432 aa, Rattus norvegicus
  CAMS
MDALEKGAATSGPAPRGRPSRGRPPKLQRSRGAGRGLEKPPHLAALVLARGGSKGIPLKNIKRLAGVPLIGWVLRAALDAGVFQSVWVSTDHDEIENVAKQFGAQVHRRSSETSKDSSTSLDAIVEFLNYHNEVDIVGNIQATSPCLHPTDLQKVAEMIREEGYDSVFSVVRRHQFRWSEIQKGVREVTEPLNLNPAKRPRRQDWDGELYENGSFYFAKRHLIEMGYLQGGKMAYYEMRAEHSVDIDVDIDWPIAEQRVLRFGYFGKEKLKEIKLLVCNIDGCLTNGHIYVSGDQKEIISYDVKDAIGISLLKKSGIEVRLISERACSKQTLSALKLDCKTEVSVSDKLAIVDEWRKEMGLCWKEVAYLGNEVSDEECLKRAGLSAVPADACSRAQKAVGYICKCNGGRGAIREFAEHIFLLLEKVNNSCQK

SEQ ID NO: 48 - DNA, 1296 bp, Rattus norvegicus
  CAMS
atggacgcgctggagaagggggccgccacgtcggggcccgccccgcgcggacggccgtcccggggccggcccccgaagctacagcgcagccggggcgcggggcgaggcctagagaagccgccgcacctggcagcgctggtgctggcccgcggcggcagcaaaggcatcccactgaagaacatcaagcgcctggcgggggttccgctcattggctgggtcctgcgcgcagccctggacgcgggtgtcttccagagtgtgtgggtttcaacagaccatgatgaaattgagaacgtggccaaacagtttggtgcccaggttcaccgaagaagttctgaaacgtccaaagacagctctacctcactagatgccatcgtagaattcctgaactatcacaatgaggttgacattgtgggaaatatccaagctacatctccatgtttacatcccacggacctccagaaagttgcagaaatgatccgagaagaaggatatgactctgtcttctccgttgtgaggcgccatcagtttcgatggagtgaaattcagaaaggagttcgtgaagtgaccgagcctctcaacctgaacccagctaagcggcctcgtcgacaggactgggatggagagctgtacgaaaacggctcgttctactttgctaagagacacttgatagagatgggctacttacagggtgggaaaatggcatattatgaaatgcgagctgagcacagtgtggacatagacgtggacatcgattggccgatcgcagagcaaagagttctgagatttggctattttgggaaagagaagctgaaggagataaagcttttggtgtgtaatattgatggatgtctcaccaatggtcacatttacgtatcaggagaccaaaaagaaataatatcttacgatgtgaaagacgctattggcataagtttactaaagaaaagtggcattgaggtgaggctcatctcagaaagggcctgctccaagcagacgctctccgccttaaagctggactgtaaaacggaagtcagtgtgtcagacaagctggccatcgtggatgagtggaggaaggagatgggcctgtgctggaaagaagtggcctatctcggcaatgaggtgtccgatgaggagtgtctgaagagagcgggcctgagcgccgtgcctgctgacgcctgctccagggcccaaaaggctgtgggctacatctgcaaatgcaacgggggccgtggagccatccgggagtttgcagaacacattttcctcctgttagaaaaggttaataactcatgccaaaaa

SEQ ID NO: 49 - protein, 432 aa, Mus musculus
  CAMS
MDALEKGAATSGPAPRGRPSRGRPPKLQRSRGAGRGLEKPPHLAALVLARGGSKGIPLKNIKRLAGVPLIGWVLRAALDAGVFQSVWVSTDHDEIENVAKQFGAQVHRRSSETSKDSSTSLDAIVEFLNYHNEVDIVGNIQATSPCLHPTDLQKVAEMIREEGYDSVFSVVRRHQFRWSEIQKGVREVTEPLNLNPAKRPRRQDWDGELYENGSFYFAKRHLIEMGYLQGGKMAYYEMRAEHSVDIDVDIDWPIAEQRVLRFGYFGKEKLKEIKLLVCNIDGCLTNGHIYVSGDQKEIISYDVKDAIGISLLKKSGIEVRLISERACSKQTLSALKLDCKTEVSVSDKLATVDEWRKEMGLCWKEVAYLGNEVSDEECLKRVGLSAVPADACSGAQKAVGYICKCSGGRGAIREFAEHIFLLIEKVNNSCQK

SEQ ID NO: 50 - DNA, 1296 bp, Mus musculus
  CAMS
atggacgcgctggagaagggggccgccacgtcggggcccgccccgcgtggacggccgtcccggggccggcccccgaagctgcagcgcagccggggcgcggggcgcggcctagagaagccgccgcacctggcagcgctggtgctggcccgcggcggcagcaaaggcatcccactgaagaacatcaagcgcctggcgggggttccgctcattggctgggtcctgcgcgccgccctggatgcgggggtcttccagagtgtgtgggtttcaacagaccatgatgaaattgagaatgtggccaaacagtttggtgcacaggtccatcgaagaagttctgaaacgtccaaagacagctctacctcactagacgccattgtagaattcctgaattatcacaatgaggttgacattgtggggaatatccaagccacatctccatgtttacatcccactgacctccagaaagttgcagaaatgatccgagaagaaggatatgactctgtcttctccgttgtgaggcgccatcagtttcgatggagtgaaattcagaaaggagttcgtgaagtgactgagcctctgaacttgaatccagcgaaacggcctcgtcgacaagactgggatggagagttatatgagaacggctcattttattttgctaaaagacatttgatagagatgggttacttacagggtgggaaaatggcatattatgaaatgcgagctgagcacagtgtggatatcgacgtggacatcgattggccgatcgcagagcaaagagttctgagatttggctattttggaaaagagaagctgaaggagataaagcttttggtttgtaatattgatggatgtctcaccaatggccacatttatgtatcaggagaccaaaaagaaataatatcttatgatgtaaaagacgctattggcataagtttattaaagaaaagcggtattgaggtgaggctcatctcagaacgggcctgctccaagcagacgctctctgccctaaagctggactgtaaaacagaagtcagtgtgtccgataagctggccaccgtggatgagtggaggaaggagatgggcctgtgctggaaagaagtggcctatctcggcaatgaagtgtctgatgaagaatgcctcaagagagtgggcctgagcgctgttcctgccgacgcctgctccggggcccagaaggctgtggggtacatctgcaaatgcagcggtggccggggagccatccgcgagtttgcagagcacattttcctactgatagaaaaagttaataactcatgccaaaaa

SEQ ID NO: 51 - protein, 406 aa, Homo sapiens
  ST6GAL1
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 52 - DNA, 1218 bp, Homo sapiens
  ST6GAL1
atgattcacaccaacctgaagaaaaagttcagctgctgcgtcctggtctttcttctgtttgcagtcatctgtgtgtggaaggaaaagaagaaagggagttactatgattcctttaaattgcaaaccaaggaattccaggtgttaaagagtctggggaaattggccatggggtctgattcccagtctgtatcctcaagcagcacccaggacccccacaggggccgccagaccctcggcagtctcagaggcctagccaaggccaaaccagaggcctccttccaggtgtggaacaaggacagctcttccaaaaaccttatccctaggctgcaaaagatctggaagaattacctaagcatgaacaagtacaaagtgtcctacaaggggccaggaccaggcatcaagttcagtgcagaggccctgcgctgccacctccgggaccatgtgaatgtatccatggtagaggtcacagattttcccttcaatacctctgaatgggagggttatctgcccaaggagagcattaggaccaaggctgggccttggggcaggtgtgctgttgtgtcgtcagcgggatctctgaagtcctcccaactaggcagagaaatcgatgatcatgacgcagtcctgaggtttaatggggcacccacagccaacttccaacaagatgtgggcacaaaaactaccattcgcctgatgaactctcagttggttaccacagagaagcgcttcctcaaagacagtttgtacaatgaaggaatcctaattgtatgggacccatctgtataccactcagatatcccaaagtggtaccagaatccggattataatttctttaacaactacaagacttatcgtaagctgcaccccaatcagcccttttacatcctcaagccccagatgccttgggagctatgggacattcttcaagaaatctccccagaagagattcagccaaaccccccatcctctgggatgcttggtatcatcatcatgatgacgctgtgtgaccaggtggatatttatgagttcctcccatccaagcgcaagactgacgtgtgctactactaccagaagttcttcgatagtgcctgcacgatgggtgcctaccacccgctgctctatgagaagaatttggtgaagcatctcaaccagggcacagatgaggacatctacctgcttggaaaagccacactgcctggcttccggaccattcactgc

SEQ ID NO: 53 - protein, 403 aa, Rattus norvegicus
  ST6GAL1
MIHTNLKKKFSLFILVFLLFAVICVWKKGSDYEALTLQAKEFQMPKSQEKVAMGSASQVVFSNSKQDPKEDIPILSYHRVTAKVKPQPSFQVWDKDSTYSKLNPRLLKIWRNYLNMNKYKVSYKGPGPGVKFSVEALRCHLRDHVNVSMIEATDFPFNTTEWEGYLPKENFRTKVGPWQRCAVVSSAGSLKNSQLGREIDNHDAVLRFNGAPTDNFQQDVGSKTTIRLMNSQLVTTEKRFLKDSLYTEGILIVWDPSVYHADIPKWYQKPDYNFFETYKSYRRLNPSQPFYILKPQMPWELWDIIQEISADLIQPNPPSSGMLGIIIMMTLCDQVDIYEFLPSKRKTDVCYYHQKFFDSACTMGAYHPLLFEKNMVKHLNEGTDEDIYLFGKATLSGFRNIRC

SEQ ID NO: 54 - DNA, 1209 bp, Rattus norvegicus
  ST6GAL1
atgattcataccaacttgaagaaaaagttcagcctcttcatcctggtctttctcctgttcgcagtcatctgtgtttggaagaaagggagcgactatgaggcccttacactgcaagccaaggaattccagatgcccaagagccaggagaaagtggccatggggtctgcttcccaggttgtgttctcaaacagcaagcaagaccctaaggaagacattccaatcctcagttaccacagggtcacagccaaggtcaaaccacagccttccttccaggtgtgggacaaggactccacatactcaaaacttaaccccaggctgctgaagatctggagaaactatctgaacatgaacaaatataaagtatcctacaagggaccggggccaggagtcaagttcagcgtagaagcactgcgttgccaccttcgagaccatgtgaacgtgtctatgatagaggccacagattttcccttcaacaccactgagtgggagggttacctgcccaaggagaactttagaaccaaggttgggccttggcaaaggtgtgccgtcgtctcttctgcaggatctctgaaaaactcccagcttggtcgagagattgataatcatgatgcagttctgaggtttaatggggcccctaccgacaacttccaacaggatgtgggctcaaaaactaccattcgcctaatgaactctcagttagtcaccacagaaaagcgcttcctcaaggacagtttgtacaccgaaggaatcctaattgtatgggacccatccgtgtatcatgcagatatcccaaagtggtatcagaaaccagactacaatttcttcgaaacctataagagttaccgaaggctgaaccccagccagccattttatatcctcaagccccagatgccatgggaactgtgggacatcattcaggaaatctctgcagatctgattcagccaaatcccccatcctccggcatgctgggtatcatcatcatgatgacgctgtgtgaccaggtagatatttacgagttcctcccatccaagcgcaagacggacgtgtgctattatcaccaaaagttctttgacagcgcttgcacgatgggtgcctaccacccgctcctcttcgagaagaatatggtgaagcatctcaatgagggaacagatgaagacatttatttgtttgggaaagccaccctttctggcttccggaacattcgttgt

SEQ ID NO: 55 - protein, 403 aa, Mus musculus
  ST6GAL1
MIHTNLKRKFSCFVLVFLLFAIICVWKKGSDYEALTLQAKVFQMPKSQEKVAVGPAPQAVFSNSKQDPKEGVQILSYPRVTAKVKPQPSLQVWDKDSTYSKLNPRLLKIWRNYLNMNKYKVSYKGPGPGVKFSVEALRCHLRDHVNVSMIEATDFPFNTTEWEGYLPKENFRTKAGPWHKCAVVSSAGSLKNSQLGREIDNHDAVLRFNGAPTDNFQQDVGTKTTIRLVNSQLVTTEKRFLKDSLYTEGILILWDPSVYHADIPQWYQKPDYNFFETYKSYRRLHPSQPFYILKPQMPWELWDIIQEISPDLIQPNPPSSGMLGIIIMMTLCDQVDIYEFLPSKRKTDVCYYHQKFFDSACTMGAYHPLLFEKNMVKHLNEGTDEDIYLFGKATLSGFRNNRC

SEQ ID NO: 56 - DNA, 1209 bp, Mus musculus
  ST6GAL1
atgattcataccaacttgaagagaaagttcagctgctttgtcctggtctttctcctgtttgccatcatctgcgtgtggaagaaagggagcgactatgaggctcttacattgcaagccaaggtattccagatgccgaagagccaggagaaagtggccgtggggcctgctccccaggctgtgttctcaaacagcaaacaagaccctaaggaaggcgttcagatcctcagttaccccagggtcacagccaaggtcaagccacagccctccttgcaggtgtgggacaaggactccacatactcaaaacttaaccccaggctgctgaagatctggaggaactatctgaacatgaataaatataaagtgtcctacaaggggccgggaccaggagtcaagttcagcgtagaggcgctgcgctgccaccttcgagaccacgtgaatgtgtctatgatagaggccacagattttcccttcaacaccactgaatgggagggttacctgcccaaggagaacttcagaaccaaggctgggccttggcataagtgtgccgtcgtgtcttctgcaggatctctgaagaactcccagctgggtcgagagattgataatcatgatgcggtcctgaggtttaatggggcacctacagacaacttccaacaggatgtgggcacaaaaactaccatccgcctagtgaactctcagttagtcaccacagaaaagcgcttcctgaaggacagtttgtacaccgaaggaatcctgattctgtgggacccatctgtgtatcatgcagacattccgcagtggtatcagaagccagactacaacttcttcgaaacctataagagttaccgaaggcttcaccccagccagcctttttacatcctcaagccccagatgccatgggaactatgggacatcattcaggaaatctctccagatctgattcagccgaatcccccatcctccggcatgctgggtatcatcattatgatgacgctgtgtgaccaagttgatatttacgagttcctcccatccaagcgcaagacagatgtgtgctactatcaccagaagttctttgacagcgcctgcacgatgggtgcctaccatccgctcctcttcgagaagaatatggtgaagcatctcaatgagggaacagatgaagacatttatttgtttgggaaagctaccctgtctggcttccggaacaatcgctgt

SEQ ID NO: 57 - protein, 337 aa, Homo sapiens
  SLC35A1
MAAPRDNVTLLFKLYCLAVMTLMAAVYTIALRYTRTSDKELYFSTTAVCITEVIKLLLSVGILAKETGSLGRFKASLRENVLGSPKELLKLSVPSLVYAVQNNMAFLALSNLDAAVYQVTYQLKIPCTALCTVLMLNRTLSKLQWVSVFMLCAGVTLVQWKPAQATKVVVEQNPLLGFGAIAIAVLCSGFAGVYFEKVLKSSDTSLWVRNIQMYLSGIIVTLAGVYLSDGAEIKEKGFFYGYTYYVWFVIFLASVGGLYTSVVVKYTDNIMKGFSAAAAIVLSTIASVMLFGLQITLTFALGTLLVCVSIYLYGLPRQDTTSIQQGETASKERVIGV

SEQ ID NO: 58 - DNA, 1011 bp, Homo sapiens
  SLC35A1
atggctgccccgagagacaatgtcactttattattcaagttatactgcttggcagtgatgaccctgatggctgcagtctataccatagctttaagatacacaaggacatcagacaaagaactctacttttcaacgacagccgtgtgtatcacagaagttataaagttattgctaagtgtgggaattttagctaaagaaactggtagtctgggtagattcaaagcatctttaagagaaaatgtcttggggagccccaaggaactgttgaagttaagtgtgccatcgttagtgtatgctgttcagaacaacatggctttcctagctcttagcaatctggatgcagcagtgtaccaggtgacctaccagttgaagattccgtgtactgctttatgcactgttttaatgttaaatcggacactcagcaaattacagtgggtttcagtttttatgctgtgtgctggagttacgcttgtacagtggaaaccagcccaagctacaaaagtggtggtggaacaaaatccattattagggtttggcgctatagctattgctgtattgtgctcaggatttgcaggagtatattttgaaaaagttttaaagagttcagatacttctctttgggtgagaaacattcaaatgtatctatcagggattattgtgacattagctggcgtctacttgtcagatggagctgaaattaaagaaaaaggatttttctatggttacacatattatgtctggtttgtcatctttcttgcaagtgttggtggcctctacacttctgttgtggttaagtacacagacaacatcatgaaaggcttttctgcagcagcggccattgtcctttccaccattgcttcagtaatgctgtttggattacagataacactcacctttgccctgggtactcttcttgtatgtgtttccatatatctctatggattacccagacaagacactacatccatccaacaaggagaaacagcttcaaaggagagagttattggtgtg

SEQ ID NO: 59 - protein, 317 aa, Rattus norvegicus
  SLC35A1
MTLVAAAYTIALRYTRTTAEGLYFSTTAVCITEVIKLLISVGLLAKETGSLGRFKASLSENVLGSPKELLKLSVPSLVYAVQNNMAFLALSNLDAAVYQVTYQLKIPCTALCTVLMLNRSLSKLQWISVFMLCGGVTLVQWKPAQATKVVVAQNPLLGFGAIAIAVLCSGFAGVYFEKVLKSSDTSLWVRNIQMYLSGIAVTLAGTYLSDGAEIKEKGFFYGYTYYVWFVIFLASVGGLYTSVVVKYTDNIMKGFSAAAAIVLSTVASVILFGLQITLSFTLGALLVCVSIYLYGLPRQDTTSIQQETTSKERIIGV

SEQ ID NO: 60 - DNA, 951 bp, Rattus norvegicus
  SLC35A1
atgactctggtggctgccgcttataccatagctttaagatatacaaggacaacagcggaaggactctacttttcaaccacagccgtgtgcatcacagaagttataaagttactgataagtgtcggccttctagctaaagaaacaggcagtttgggtagatttaaagcctctttgagtgaaaacgtcttggggagccctaaggagctgctgaagttaagtgtgccgtcactggtgtatgctgtgcagaacaacatggctttcctagctctcagtaacctggatgcagcagtgtaccaggtgacctatcaactgaagattccctgcactgctttatgtactgttttaatgttaaatcgatcactcagcaaactacagtggatttcggtcttcatgctgtgtggtggggtcacacttgtacagtggaaaccagcccaagctacaaaagtcgtggtagcgcagaacccgttgttaggctttggagctatagccattgctgtgctgtgctcgggatttgcaggagtttattttgaaaaagttttaaagagttcagacacttccctttgggtgagaaacattcagatgtatctgtcagggatcgctgtgacattagctggtacctacttgtcggatggcgctgaaattaaagaaaaaggatttttctatggctacacgtattatgtctggtttgttatcttccttgctagtgtgggaggcctctacacgtcagtggtggtgaagtacacagacaacatcatgaaaggcttctctgcggccgcagccattgttctgtctaccgttgcctcagtcatactgtttggattgcagataacactttcatttacactgggagctcttcttgtatgtgtttccatttatctctatgggttacccagacaagataccacatccattcaacaagaaacaacttcaaaagaaagaatcattggtgtg

SEQ ID NO: 61 - protein, 336 aa, Mus musculus
  SLC35A1
MAPARENVSLFFKLYCLTVMTLVAAAYTVALRYTRTTAEELYFSTTAVCITEVIKLLISVGLLAKETGSLGRFKASLSENVLGSPKELAKLSVPSLVYAVQNNMAFLALSNLDAAVYQVTYQLKIPCTALCTVLMLNRTLSKLQWISVFMLCGGVTLVQWKPAQATKVVVAQNPLLGFGAIAIAVLCSGFAGVYFEKVLKSSDTSLWVRNIQMYLSGIVVTLAGTYLSDGAEIQEKGFFYGYTYYVWFVIFLASVGGLYTSVVVKYTDNIMKGFSAAAAIVLSTIASVLLFGLQITLSFALGALLVCVSIYLYGLPRQDTTSIQQEATSKERIIGV

SEQ ID NO: 62 - DNA, 1008 bp, Mus musculus
  SLC35A1
atggctccggcgagagaaaatgtcagtttattcttcaagctgtactgcttgacggtgatgactctggtggctgccgcttacaccgtagctttaagatacacaaggacaacagctgaagaactctacttctcaaccactgccgtgtgtatcacagaagtgataaagttactgataagtgttggcctgttagctaaggaaactggcagtttgggtagatttaaagcctcattaagtgaaaatgtcttggggagccccaaggaactggcgaagttgagtgtgccatcactagtgtatgctgtgcagaacaacatggccttcctggctctcagtaatctggatgcagcagtgtaccaggtgacctatcaactgaagatcccctgcactgctttatgtactgttttaatgttaaatcgaacactcagcaaattacagtggatttccgtcttcatgctgtgtggtggggtcacactcgtacagtggaaaccagcccaagctacaaaagtcgtggtagcgcagaatccattgttaggctttggtgctatagctattgctgtattgtgctctggatttgcaggagtttattttgaaaaagtcttaaagagttccgacacttccctttgggtgagaaacattcagatgtatctgtcagggatcgttgtgacgttagctggtacctacttgtcagatggagctgaaattcaagaaaaaggattcttctatggctacacgtattatgtctggtttgttatcttccttgctagtgtgggaggcctctacacgtcagtggtggtgaagtatacagacaacatcatgaaaggcttctctgctgccgcagccattgttctttctaccattgcttcagtcctactgtttggattacagataacactttcatttgcactgggagctcttcttgtgtgtgtttccatatatctctatgggttacccagacaagatactacatccattcaacaagaagcaacttcaaaagagagaatcattggtgtg

SEQ ID NO: 63 - DNA, 27 bp, Artificial
  primer
ggcgcgccctttgtatccctttttacg

SEQ ID NO: 64 - DNA, 25 bp, Artificial
  primer
ggatccgatcgcttgatccttagag

SEQ ID NO: 65 - DNA, 30 bp, Artificial
  primer
gtacgtaagcttgatcaaacttcgttttcg

SEQ ID NO: 66 - DNA, 44 bp, Artificial
  primer
gcctagggagacggcagatcgtctcccatgttggcggtctttgg

SEQ ID NO: 67 - DNA, 30 bp, Artificial
  primer
gtcatgagagtcaaaacctttgtgatcttg

SEQ ID NO: 68 - DNA, 45 bp, Artificial
  primer
gcctagggagacggcagatcgtctcctgcatttgtataagcgaca

SEQ ID NO: 69 - DNA, 13 bp, Artificial
  primer
cgcgccgctagcc

SEQ ID NO: 70 - DNA, 13 bp, Artificial
  primer
tcgaggctagcgg

SEQ ID NO: 71 - DNA, 35 bp, Artificial
  primer
gtcatgaggtttcgtgagcagttcctgggcggcag

SEQ ID NO: 72 - DNA, 40 bp, Artificial
  primer
gcctaggttatctcggtgtcccgatgtccactgtgatttg

SEQ ID NO: 73 - DNA, 40 bp, Artificial
  primer
gcgtctcccatgagcagactgctgggggggacgctggagc

SEQ ID NO: 74 - DNA, 40 bp, Artificial
  primer
gcgtctccctaggtcagccttgagtgagccacgacatggg

SEQ ID NO: 75 - DNA, 44 bp, Artificial
  primer
gcgtctcccatgttgcggaggctgctggagagaccctgtacatt

SEQ ID NO: 76 - DNA, 45 bp, Artificial
  primer
gcgtctccctaggttagtgtgagccacggggagctgtgtggttgg

SEQ ID NO: 77 - DNA, 35 bp, Artificial
  primer
gccatgggctgcaacccaccttatcacctctccta

SEQ ID NO: 78 - DNA, 40 bp, Artificial
  primer
gcctaggttacgcagcagtccagaaatccactgtgatgtt

SEQ ID NO: 79 - DNA, 27 bp, Artificial
  primer
ggctagcgcatgcaagcttgagctcga

SEQ ID NO: 80 - DNA, 27 bp, Artificial
  primer
gactagtaagcttatcgataccgtcga

SEQ ID NO: 81 - DNA, 30 bp, Artificial
  primer
gcgtctcccatggagaagaacgggaataac

SEQ ID NO: 82 - DNA, 30 bp, Artificial
  primer
ccgtctccctaggctagtggatcctgcggg

SEQ ID NO: 83 - DNA, 30 bp, Artificial
  primer
gcgtctcccatgccgctggagctggagctg

SEQ ID NO: 84 - DNA, 30 bp, Artificial
  primer
ccgtctccctagtcaagacttgattttttt

SEQ ID NO: 85 - DNA, 30 bp, Artificial
  primer
gcgtctcccatggggctgagccgcgtgcgg

SEQ ID NO: 86 - DNA, 30 bp, Artificial
  primer
cgtctccctagtcaagtggacatactgact

SEQ ID NO: 87 - DNA, 30 bp, Artificial
  primer
gcgtctcccatggactcggtggagaagggg

SEQ ID NO: 88 - DNA, 30 bp, Artificial
  primer
ccgtctccctaggctatttttggcatgaat

SEQ ID NO: 89 - DNA, 30 bp, Artificial
  primer
gcgtctcccatggctgccccgagagacaat

SEQ ID NO: 90 - DNA, 30 bp, Artificial
  primer
ccgtctccctagtcacacaccaataactct

SEQ ID NO: 91 - DNA, 30 bp, Artificial
  primer
gcgtctcccatgattcacaccaacctgaag

SEQ ID NO: 92 - DNA, 30 bp, Artificial
  primer
ccgtctccctaggttagcagtgaatggtcc

SEQ ID NO: 93 - DNA, 120 bp, Artificial
  synthetic
gctagcaggcgcgccgggatgtaattacgtccctcccccgctagggggcagcagcgagccgcccggggctccgctccggtccggcgctccccccgcatccccgagggcgcgcctactagt

SEQ ID NO: 94 - DNA, 19 bp, Artificial
  primer
gtctagaactagtgctagc

SEQ ID NO: 95 - DNA, 23 bp, Artificial
  primer
gctagcactagttctagactgca

SEQ ID NO: 96 - DNA, 40 bp, Artificial
  primer
gcgtctcatgcacacgggagccctgtggacatctgcacag

SEQ ID NO: 97 - DNA, 45 bp, Artificial
  primer
cgcgtctccctaggttagtgatgatgatggtgatgcttaacacaa

SEQ ID NO: 98 - DNA, 39 bp, Artificial
  primer
gcgtctcatgcacaggacccatatgtaaaagaagcagaa

SEQ ID NO: 99 - DNA, 45 bp, Artificial
  primer
cgcgtctccctaggttagtgatgatgatggtgatgctgggatgct